(19) United States
(12) Patent Application Publication
(10) Pub. No.: US 2008/0227796 A1
(43) Pub. Date: Sep. 18, 2008

(54) 7-Amino-6-Hetaryl-1,2,4-Triazolo[1,5-A]Pyrimidine Compounds and the Use Thereof for Controlling Pathogenic Fungi

(75) Inventors: Oliver Wagner, Neustadt (DE); Thomas Grote, Wachenheim (DE); Joachim Rheinheimer, Ludwigshafen (DE); Barbara Nave, Deidesheim (DE); Reinhard Stierl, Freinsheim (DE)

Correspondence Address: BRINKS, HOFER, GILSON & LIONE, 2801 SLATER ROAD, SUITE 120, MORRISVILLE, NC, 27560, US

(73) Assignee: BASF Aktiengesellschaft, Ludwigshafen (DE)

(21) Appl. No.: 11/793,286

(22) PCT Filed: Dec. 16, 2005
(86) PCT No.: PCT/EP05/13577

§ 371 Date: Jun. 14, 2007

(30) Foreign Application Priority Data
Dec. 17, 2004 (DE) 102004060958.6
Dec. 23, 2004 (DE) 102004062199.3

(51) Int. Cl. A01N 43/90; C07D 487/04; A01P 3/00

(52) U.S. Cl. 514/259.31; 544/263; 514/255.05

(57) ABSTRACT

The present invention relates to novel 7-amino-6-hetaryl-1,2,4-triazolo[1,5-a]pyrimidine compounds and to their agriculturally acceptable salts, and also to their use for controlling harmful fungi, and also to crop protection compositions comprising at least one such compound as active component.

The novel 7-amino-6-hetaryl-1,2,4-triazolo[1,5-a]pyrimidine compounds can be described by the formula I

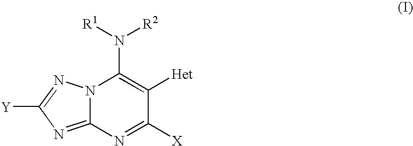

where the substituents R¹, R², Het, X and Y in formula I are as defined below:
Het is a 6-membered heteroaromatic radical selected from the group consisting of pyridinyl, pyridazinyl, pyrazinyl, 1,2,4-triazinyl and 1,3,5-triazinyl, where the 6-membered heteroaromatic radical may have 1, 2 or 3 identical or different substituents L,
R¹, R² independently of one another are hydrogen, C₁-C₈-alkyl, C₁-C₈-haloalkyl, C₁-C₈-alkoxy, C₃-C₈-cycloalkyl, C₃-C₈-cycloalkoxy, C₅-C₁₀-bicycloalkyl, C₃-C₈-halocycloalkyl, C₂-C₈-alkenyl, C₂-C₈-alkenyloxy, C₄-C₁₀-alkadienyl, C₂-C₈-haloalkenyl, C₃-C₈-cycloalkenyl, C₃-C₈-halocycloalkenyl, C₂-C₈-alkynyl, C₂-C₈-alkynyloxy, C₂-C₈-haloalkynyl, NH₂, C₁-C₈-alkylamino, di-C₁-C₈-alkylamino, phenyl, naphthyl, or a five- or six-membered saturated, partially unsaturated or aromatic heterocycle,
X is hydrogen, halogen, OH, cyano, C₁-C₄-alkyl and the like,
Y is hydrogen, halogen, cyano, C₁-C₄-alkyl and the like.

The present invention relates to novel 7-amino-6-hetaryl-1,2,4-triazolo[1,5-a]pyrimidine compounds and to their use for controlling harmful fungi, and also to crop protection compositions comprising at least one such compound as active component.

EP-A 71 792, EP-A 550 113, EP-A 834 513 and WO-A 98/46608 describe fungicidally active 1,2,4-triazolo[1,5-a]pyrimidines which carry an optionally substituted phenyl ring in the 6-position, a halogen atom in the 5-position and an amino group in the 7-position.

WO 02/50077 describes similar 1,2,4-triazolo[1,5-a]pyrimidines which may furthermore carry a heterocyclic radical in the 6-position. Compounds carrying a 6-membered heteroaromatic radical in this position are not described.

EP-A 613 900 describes fungicidally active 1,2,4-triazolo[1,5-a]pyrimidine compounds which have a hydrogen atom or halogen atom in the 5-position and a secondary or tertiary amino group in the 7-position. These compounds have a cycloalkyl radical or heterocyclyl radical, for example a 3-thienyl radical, in the 6-position. Compounds carrying a 6-membered heteroaromatic radical in this position are not described.

WO 04/0011467 in turn describes fungicidally active 1,2,4-triazolo[1,5-a]pyrimidine compounds which have a halogen atom, a cyano group, an alkoxy group, an alkylthio group, an alkylsulfinyl group, an alkylsulfonyl group, an alkylamino group or an alkoxycarbonyl group in the 5-position. In the 7-position, these compounds may, inter alia, carry a tertiary amino group. In the 6-position, these compounds have a 5- or 6-membered heterocyclyl radical selected from the group consisting of optionally substituted pyrrolyl, thienyl, pyrazolyl, imidazolyl, oxazolyl, isoxazolyl, thiazolyl, isothiazolyl and pyrimidyl.

Some of the 1,2,4-triazolo[1,5-a]pyrimidines known from the prior art are, with respect to their fungicidal action, not satisfactory, or they have unwanted properties, such as low crop plant compatibility.

Accordingly, it is an object of the present invention to provide novel compounds having improved fungicidal activity and/or better crop plant compatibility.

Surprisingly, this object is achieved by 7-amino-6-hetaryl-1,2,4-triazolo[1,5-a]pyrimidine compounds of the formula I defined below, and by the agriculturally acceptable salts of the compounds I.

Accordingly, the present invention relates to 7-amino-6-hetaryl-1,2,4-triazolo-[1,5-a]pyrimidine compounds of the formula I

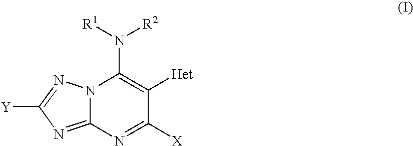

and to the agriculturally acceptable salts of compounds of the formula I where the substituents R¹, R², Het, X and Y in formula I are as defined below:
Het is a 6-membered heteroaromatic radical selected from the group consisting of pyridinyl, pyridazinyl, pyrazinyl, 1,2,4-triazinyl and 1,3,5-triazinyl, where the 6-membered heteroaromatic radical may have 1, 2, 3 or 4, preferably 1, 2 or 3 identical or different substituents L,
R¹, R² independently of one another are hydrogen, C₁-C₈-alkyl, C₁-C₈-haloalkyl, C₁-C₈-alkoxy, C₃-C₈-cycloalkyl, C₃-C₈-cycloalkoxy, C₅-C₁₀-bicycloalkyl, C₃-C₈-halocycloalkyl, C₂-C₈-alkenyl, C₂-C₈-alkenyloxy, C₄-C₁₀-alkadienyl, C₂-C₈-haloalkenyl, C₃-C₆-cycloalkenyl, C₃-C₆-halocycloalkenyl, C₂-C₈-alkynyl, C₂-C₈-alkynyloxy, C₂-C₈-haloalkynyl, NH₂, C₁-C₈-alkylamino, di-C₁-C₈-alkylamino, phenyl, naphthyl or a five- or six-membered saturated, partially unsaturated or aromatic heterocycle which contains one, two or three or four heteroatoms from the group consisting of O, N and S, where preferably at least one of the radicals R¹ or R² is different from hydrogen,
   R¹ and R² together with the nitrogen atom to which they are attached may also form a five- or six-membered heterocyclyl or heteroaryl which is attached via N and may contain one, two or three further heteroatoms from the group consisting of O, N and S as ring members and/or may carry one or more substituents, for example, 1, 2, 3 or 4 substituents, from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy and/or in which two substituents attached to adjacent ring atoms may be C₁-C₆-alkylene, oxy-C₂-C₄-alkylene or oxy-C₁-C₃-alkyleneoxy;
   R¹ and/or R² may carry one, two, three or four identical or different groups R^{a}:
   R^{a} is cyano, nitro, hydroxyl, carboxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₈-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five-, six-, seven-, eight-, nine- or ten-membered saturated, partially unsaturated or aromatic heterocycle which contains one, two, three or four heteroatoms from the group consisting of O, N and S,
      where the aliphatic, alicyclic or aromatic groups in R^{a} for their part may be partially or fully halogenated or may carry one, two or three groups R^{b}:
      R^{b} is cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
         and/or one, two or three of the following radicals:
         cycloalkyl, bicycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6, 7, 8, 9 or 10 ring members and the hetaryl radicals 5 or 6 ring members, where the cyclic systems may be partially or fully halogenated or substituted by alkyl or haloalkyl groups;
X is hydrogen, OH, halogen, cyano, NR³R⁴, C₁-C₈-alkyl, C₁-C₈-alkoxy, C₁-C₈-alkylthio, C₁-C₈-alkylsulfinyl, C₁-C₈-alkylsulfonyl, C₂-C₈-alkenyl or C₂-C₈-alkynyl, where the 7 last-mentioned radicals may be partially or fully halogenated and/or may carry one, two or three substituents selected from the group consisting of nitro, cyano, C₁-C₂-alkoxy, C₁-C₄-alkoxycarbonyl, amino, C₁-C₄-alkylamino and di-C₁-C₄-alkylamino, and where R³ and R⁴ independently of one another have the meanings given for R¹ and R², respectively;
Y is hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, formyl, C₁-C₄-alkylcarbonyl, C₁-C₄-alkoxycarbonyl, CONR³R⁴ or C₁-C₂-haloalkoxy; where
L is selected from the group consisting of halogen, cyano, hydroxyl, cyanato (OCN), nitro, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoximinoalkyl, C₂-C₁₀-alkenyloximinoalkyl, C₂-C₁₀-alkynyloximinoalkyl, C₂-C₁₀-alkynylcarbonyl, C₃-C₆-cycloalkylcarbonyl, phenyl, a five-, six-, seven-, eight-, nine- or ten-membered saturated, partially unsaturated or aromatic heterocycle which contains one, two, three or four heteroatoms from the group consisting of O, N and S, where phenyl and the heterocycle may be unsubstituted or have 1, 2, 3 or 4 substituents selected from the group consisting of halogen and R^{a}, amino, NR⁵R⁶, NR⁵—(C═O)—R^{a}, S(═O)ₙA¹, C(═O)A², C(═S)A², a group —C(═N—OR⁷)(NR⁸R⁹) and a group —C(═N—NR¹⁰R¹¹)(NR¹²R¹³),
   where
   R⁵, R⁶ independently of one another are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, where the 5 last-mentioned radicals may be partially or fully halogenated or may carry one, two, three or four radicals selected from the group consisting of cyano, C₁-C₄-alkoximino, C₂-C₄-alkenyloximino, C₂-C₄-alkynyloximino and C₁-C₄-alkoxy;
   A¹ is hydrogen, hydroxyl, C₁-C₈-alkyl, amino, C₁-C₈-alkylamino or di-(C₁-C₈-alkyl)amino;
   n is 0, 1 or 2;
   A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A¹;
   R⁷, R⁸, R⁹, R¹⁰, R¹¹, R¹² and R¹³ independently of one another are selected from the group consisting of hydrogen, C₁-C₆-alkyl, C₃-C₆-cycloalkyl, C₂-C₆-alkenyl and C₂-C₆-alkynyl, where the four last-mentioned radicals may have one, two, three, four, five or six radicals R^{a}; or
   R⁸ and R⁹, R¹⁰ and R¹¹ and/or R¹² and R¹³ together with the nitrogen atom to which they are attached form a four-, five- or six-membered saturated or partially unsaturated ring which may carry one, two, three or four substituents independently of one another selected from the group R^{a}.

The invention furthermore provides compounds of the formula I and salts thereof where R¹, Het, X and Y are as defined above and R² is an organic radical which contains 3 to 13 carbon atoms and one or more, for example 1, 2 or 3, silicon atoms, and optionally 1 to 3 identical or different heteroatoms from the group consisting of oxygen, nitrogen and sulfur, and which is unsubstituted or carries 1, 2, 3 or 4 identical or different substituents selected from the group consisting of halogen atoms and the substituents R^{a}. The invention furthermore provides compounds of the formula I in which Het, X and Y are as defined above and in which R¹ and R² together with the nitrogen atom to which they are attached are a heterocyclic ring which preferably has 3 to 12 ring members, which has one or more, for example 1, 2 or 3 silicon atoms and which is unsubstituted or carries 1, 2, 3 or 4 identical or different substituents selected from the group consisting of halogen atoms and the substituents R^{a}.

The present invention furthermore provides the use of the 7-amino-1,2,4-triazolo-[1,5-a]pyrimidine compounds of the formula I and their agriculturally acceptable salts for controlling phytopathogenic fungi (=harmful fungi), and a method for controlling phytopathogenic harmful fungi which comprises treating the fungi or the materials, plants, the soil or seed to be protected against fungal attack with an effective amount of at least one compound of the formula I and/or an agriculturally acceptable salt of I.

The present invention furthermore provides a composition for controlling harmful fungi, which composition comprises at least one compound of the formula I and/or an agriculturally acceptable salt thereof and at least one liquid or solid carrier.

Depending on the substitution pattern, the compounds of the formula I may have one or more centers of chirality, in which case they are present as mixtures of enantiomers or diastereomers. The invention provides both the pure enantiomers or diastereomers and their mixtures. Suitable compounds of the formula I also include all possible stereoisomers (cis/transisomers) and mixtures thereof.

Suitable agriculturally useful salts are especially the salts of those cations or the acid addition salts of those acids whose cations and anions, respectively, have no adverse effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium iron which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

In the definitions of the variables given in the formulae above, collective terms are used which are generally representative for the substituent in question. The term Cₙ-Cₘ indicates the number of carbon atoms possible in each case in the substituent or substituent moiety in question:

halogen: fluorine, chlorine, bromine and iodine;
alkyl and the alkyl moieties in alkyloxy, alkylthio, alkylsulfinyl and alkylsulfonyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl and the like;
haloalkyl: straight-chain or branched alkyl groups having 1 to 2, 4, 6 or 8 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl and the alkenyl moieties in alkenyloxy: monounsaturated straight-chain or branched hydrocarbon radicals having 2 to 4, 2 to 6, 2 to 8 or 2 to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like;
alkadienyl: diunsaturated straight-chain or branched hydrocarbon radicals having 4 to 10 carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;
haloalkenyl: unsaturated straight-chain or branched hydrocarbon radicals having 2 to 10 carbon atoms and a double bond in any position (as mentioned above), where some or all of the hydrogen atoms in these groups may be replaced by halogen atoms as mentioned above, in particular fluorine, chlorine and bromine;
alkynyl and the alkynyl moieties in alkynyloxy: straight-chain or branched hydrocarbon groups having 2 to 4, 2 to 6, 2 to 8 or 2 to 10 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;
cycloalkyl and the cycloalkyl moieties in cycloalkoxy: monocyclic saturated hydrocarbon groups having 3 to 8 carbon ring members, such as cyclopropyl, cyclobutyl, cyclopentyl, cyclohexyl, cycloheptyl and cyclooctyl;
cycloalkenyl: monocyclic monounsaturated hydrocarbon groups having 3 to 8, preferably 5 to 6, carbon ring members, such as cyclopenten-1-yl, cyclopenten-3-yl, cyclohexen-1-yl, cyclohexen-3-yl, cyclohexen-4-yl and the like;
bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like;
C₁-C₄-alkoxy: an alkyl group having 1 to 4 carbon atoms which is attached via an oxygen, for example, methoxy, ethoxy, n-propoxy, 1-methylethoxy, butoxy, 1-methylpropoxy, 2-methylpropoxy or 1,1-dimethylethoxy;
C₁-C₆-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;
C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;
C₁-C₆-haloalkoxy: C₁-C₄-haloalkoxy as mentioned above and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;
alkenyloxy: alkenyl as mentioned above which is attached via an oxygen atom, for example C₃-C₆-alkenyloxy, such as 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;
alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;
alkylthio: alkyl, as defined above which is attached via a sulfur atom;
alkylsulfinyl: alkyl as defined above which is attached via an SO group;
alkylsulfonyl: alkyl as defined above which is attached via an S(O)₂ group;
a 5-, 6-, 7-, 8-, 9- or 10-membered saturated, partially unsaturated or aromatic heterocycle which contains 1, 2, 3 or 4 heteroatoms from the group consisting of oxygen, nitrogen and sulfur:
a five- or six-membered saturated or partially unsaturated heterocycle (hereinbelow also referred to as heterocyclyl) which contains one, two, three or four heteroatoms from the group consisting of oxygen, nitrogen and sulfur as ring members: for example monocyclic saturated or partially unsaturated heterocycles which contain, in addition to carbon ring members, one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding-ylidene radicals;
a seven-membered saturated or partially unsaturated heterocycle which contains one, two, three or four heteroatoms from the group consisting of oxygen, nitrogen and sulfur as ring members: for example mono- and bicyclic heterocycles having 7 ring members which contain, in addition to carbon ring members, one to three nitrogen atoms and/or one oxygen or sulfur atom or one or two oxygen and/or sulfur atoms, for example tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl,
hexahydroazepin-1-, -2-, -3- or -4-yl, tetra-.and hexahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra-.and hexahydro-1,3-diazepinyl, tetra- and hexahydro-1,4-diazepinyl, tetra-.and hexahydro-1,3-oxazepinyl, tetra-.and hexahydro-1,4-oxazepinyl, tetra-.and hexahydro-1,3-dioxepinyl, tetra- and hexahydro-1,4-dioxepinyl and the corresponding -ylidene radicals;
a five- or six-membered aromatic heterocycle which contains one, two, three or four heteroatoms from the group consisting of oxygen, nitrogen and sulfur: mono- or bicyclic heteroaryl, for example 5-membered heteroaryl which is attached via carbon and contains one to three nitrogen atoms or one or two nitrogen atoms and/or one sulfur or oxygen atom as ring members, such as 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 5-membered heteroaryl which is attached via nitrogen and contains one to three nitrogen atoms as ring members, such as pyrrol-1-yl, pyrazol-1-yl, imidazol-1-yl, 1,2,3-triazol-1-yl and 1,2,4-triazol-1-yl; 6-membered heteroaryl which contains one to three nitrogen atoms as ring members, such as pyridin-2-yl, pyridin-3-yl, pyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl;
   alkylene: divalent unbranched chains of 1 to 6 CH₂ groups, for example CH₂, CH₂CH₂, CH₂CH₂CH₂, CH₂CH₂CH₂CH₂, CH₂CH₂CH₂CH₂CH₂ and CH₂CH₂CH₂CH₂CH₂CH₂;
   oxyalkylene: divalent unbranched chains of 2 to 4 CH₂ groups, where one valency is attached to the skeleton via an oxygen atom, for example OCH₂CH₂, OCH₂CH₂CH₂ and OCH₂CH₂CH₂CH₂;
   oxyalkyleneoxy: divalent unbranched chains of 1 to 3 CH₂ groups, where both valences are attached to the skeleton via an oxygen atom, for example OCH₂O, OCH₂CH₂O and OCH₂CH₂CH₂O.

Examples of organic radicals which contain 3 to 13 carbon atoms and one or more silicon atoms and optionally 1 to 3 identical or different heteroatoms from the group consisting of oxygen, nitrogen and sulfur and which are unsubstituted or may carry 1 to 4 identical or different halogen atoms are SiMe₃, SiMe₂Et, SiMe₂CHMe₂, SiMe₂CH₂CHMe₂, SiMe₂CH₂CMe₃, SiMe₂OCHMe₂, SiMe₂OCH₂CHMe₂, CH₂SiMe₃, CH₂SiMe₂Et, CH₂SiMe₂CHMe₂, CH₂SiMe₂CH₂CHMe, CH₂SiMe₂OMe, CH₂SiMe₂OCHMe₂, CH₂SiMe₂OCH₂CHMe₂, CHMeSiMe₃, CHMeSiMe₂OMe, (CH₂)₂SiMe₃, (CH₂)₂SiMe₂Et, (CH₂)₂SiMe₂CHMe₂, (CH₂)₂SiMe₂CMe₃, (CH₂)₂SiMe₂CH₂CHMe₂, (CH₂)₂SiMe₂CH₂CH₂Me, (CH₂)₂SiMe₂CH₂CMe₃, (CH₂)₂SiMe₂OCHMe₂, (CH₂)₂SiMe₂OCH₂CHMe₂, CHMeCH₂SiMe₃, CHMeCH₂SiMe₂Et, CHMeCH₂SiMe₂CH₂CH₂Me, CHMeCH₂—SiMe₂CHMe₂, CHMeCH₂SiMe₂CMe₃, CHMeCH₂SiMe₂CH₂CHMe₂, CFMeCH₂SiMe₃, CHMeCH₂CH₂SiMe₂OMe, CHMeCH₂SiMe₂OCHMe₂, CHMeCH₂SiMe₂OCH₂CHMe₂, CH₂CHMeSiMe₃, CH₂CHMeSiMe₂Et, CH₂CHMeSiMe₂CHMe₂, CHMeCHMeSiMe₃, CMe₂CH₂SiMe₃, (CH₂)₃SiMe₃, (CH₂)₃SiMe₂Et, (CH₂)₃Si-Me₂CHMe₂, (CH₂)₃SiMe₂CH₂CHMe₂, (CH₂)₃SiMe₂OMe, (CH₂)₃SiMe₂OCHMe₂, (CH₂)₃SiMe₂OCH₂CHMe₂, CHMeCH₂CH₂SiMe₃, CHMeCH₂CH₂SiMe₂Et, CHMeCH₂CH₂SiMe₂CHMe₂, CHMeCH₂CH₂CH₂SiMe₂OMe, CHMeCH₂—CH₂SiMe₂OCHMe₂, CMe=CHSiMe₃, CH₂CH₂SiMe₂OMe, —C≡C—SiMe₃, —CH₂—C≡C—SiMe₃ and —CHMe-C≡C—SiMe₃, where Me is methyl and Et is ethyl.

With a view to the fungicidal activity, preference is given to compounds of the formula I in which at least one of the radicals R¹ or R² is different from hydrogen. Among these, preference is given to compounds of the formula I in which R¹ is different from hydrogen and R² is hydrogen. Preference is likewise given to compounds of the formula I in which R¹ and R² are different from hydrogen. Among these, preference is given to compounds of the formula I in which R² is C₁-C₄-alkyl, especially methyl or ethyl.

For the fungicidal action of the compounds according to the invention, it is furthermore advantageous if the substituents R¹, X and Y independently of one another and preferably in combination have particularly preferably the meanings given below:
R¹ is in particular C₁-C₆-alkyl, C₂-C₆-alkenyl, C₂-C₆-alkynyl, C₃-C₆-cycloalkyl which may be mono-, di-, tri- or tetrasubstituted by halogen or C₁-C₄-alkyl, or C₁-C₈-haloalkyl.
Among these, a particularly preferred embodiment relates to compounds of the formula I, in which R¹ is a group B:

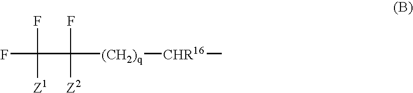

in which
Z¹ is hydrogen, fluorine or C₁-C₄-fluoroalkyl,
Z² is hydrogen or fluorine, or
Z¹ and Z² together form a double bond;
q is 0 or 1; and
R¹⁶ is hydrogen or methyl.
Among these, particular preference is furthermore given to compounds of the formula I in which R¹ is C₃-C₆-cycloalkyl which may be substituted by C₁-C₄-alkyl.
Preference is likewise given to compounds of the formula I in which R¹ and R² together with the nitrogen atom to which they are attached are saturated or monounsaturated, in particular 5- or 6-membered heterocyclyl as defined above. Among these, preference is given to those compounds in which R¹ and R² together with the nitrogen atom to which they are attached form an optionally substituted piperidinyl, morpholinyl or thiomorpholinyl ring, especially a piperidinyl ring. Heterocyclyl is in particular unsubstituted or substituted by 1, 2 or 3 substituents R^{a}, preferred substitutents R^{a} on heterocyclyl being selected from the group consisting of halogen, C₁-C₄-alkyl and C₁-C₄-haloalkyl. Among these, preference is given in particular to compounds I in which R¹ and R² together with the nitrogen atom to which they are attached form a 4-methylpiperidine ring, a 4-trifluoromethylpiperidine ring, a morpholine ring or a 3,4-dimethylpiperidine ring and especially a 4-methylpiperidine ring or a 3,4-dimethylpiperidine ring.
The invention furthermore particularly preferably provides compounds I in which R¹ and R² together with the nitrogen atom to which they are attached are 5- or 6-membered heteroaryl as defined above which may be unsubstituted or substituted, preferably by 1, 2 or 3 groups R^{a}. In this case, the group NR¹R² forms in particular a pyrazole ring which is optionally substituted in the manner described above and especially by 1 or 2 of the following radicals: halogen, C₁-C₄-alkyl or C₁-C₄-haloalkyl, in particular by 2 methyl groups or 2 trifluoromethyl groups in the 3,5-position.
Very particular preference is given to compounds of the formula I in which R¹ is selected from the group consisting of: CH(CH₃)—CH₂CH₃, CH(CH₃)—CH(CH₃)₂, CH(CH₃)—C(CH₃)₃, CH(CH₃)—CF₃, CH₂C(CH₃)═CH₂, CH₂CH═CH₂, cyclopentyl or cyclohexyl; and R² is hydrogen or methyl and also compounds I in which R¹ and R² together are —(CH₂)₂CH(CH₃)(CH₂)₂—, —(CH₂)₂CH(CF₃)(CH₂)₂— or —(CH₂)₂—O—(CH₂)₂—.
In a first embodiment of the invention, X is hydrogen, OH, halogen, cyano, NR³R⁴, C₁-C₈-alkoxy, C₁-C₈-alkylthio, C₁-C₈-alkylsulfinyl, C₁-C₈-alkylsulfonyl, where the 4 last-mentioned radicals may be partially or fully halogenated and/or may carry one, two or three substituents selected from the group consisting of nitro, cyano, C₁-C₂-alkoxy, C₁-C₄-alkylthio, C₁-C₄-alkoxycarbonyl, amino, C₁-C₄-alkylamino and di-C₁-C₄-alkylamino and in which R³ and R⁴ independently of one another have the meanings given for R¹ and R². In this embodiment, X is in particular halogen, cyano, C₁-C₄-alkoxy or C₁-C₂-haloalkoxy, particularly preferably halogen, cyano or C₁-C₂-alkoxy, such as chlorine, cyano, methoxy or ethoxy, particularly preferably halogen and especially chlorine. In a second embodiment of the invention, X is C₁-C₈-alkyl, C₂-C₈-alkenyl or C₂-C₈-alkinyl, where the 3 radicals may be partially or fully halogenated and/or may carry one, two or three substituents selected from the group consisting of nitro, cyano, C₁-C₂-alkoxy, C₁-C₄-alkoxycarbonyl, amino, C₁-C₄-alkylamino and di-C₁-C₄-alkylamino. In this embodiment, X is in particular C₁-C₄-alkyl, C₁-C₄-haloalkyl, particularly preferably C₁-C₂-alkyl, such as methyl or ethyl, or C₁-C₂-haloalkyl, such as, for example, fluoromethyl, chloromethyl, bromomethyl, difluoromethyl, dichloromethyl, trifluoromethyl, trichloromethyl, chlorofluoromethyl, dichlorofluoromethyl or chlorodifluoromethyl, and especially methyl. X is in particular halogen, cyano, C₁-C₄-alkyl, C₁-C₄-haloalkyl, C₁-C₄-alkoxy or C₁-C₂-haloalkoxy. Particular preference is given to compounds of the formula I in which X is halogen, C₁-C₂-alkyl, cyano or C₁-C₂-alkoxy, such as chlorine, methyl, cyano, methoxy or ethoxy. X is in particular halogen and especially chlorine. Preference is in particular also given to compounds of the formula I in which X is CN, C₁-C₄-alkoxy or C₁-C₄-alkylthio, especially CN, methoxy or methylthio.
Y is preferably hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl or C₁-C₂-haloalkoxy and in particular hydrogen.
Preferred substituents L on Het are halogen, cyano, nitro, NH₂, C₁-C₆-alkylamino, di-C₁-C₆-alkylamino, C₁-C₆-alkyl, C₁-C₆-haloalkyl, C₁-C₆-alkoxy, C₁-C₆-alkylamino, di-C₁-C₆-alkylamino, NH—C(O)—C₁-C₆-alkyl, a group C(S)A² and a group C(O)A². Here, A² has the meanings given above and preferably C₁-C₄-alkoxy, NH₂, C₁-C₄-alkylamino or di-C₁-C₄-alkylamino. Particularly preferred radicals L are, independently of one another, selected from the group consisting of fluorine, chlorine, bromine, iodine, cyano, nitro, C₁-C₄-alkyl, especially methyl, C₁-C₄-haloalkyl, C₁-C₄-alkoxy and C₁-C₄-alkoxycarbonyl, particularly preferably from the group consisting of fluorine, chlorine, C₁-C₂-alkyl, such as methyl or ethyl, C₁-C₂-fluoroalkyl, such as trifluoromethyl, C₁-C₂-alkoxy such as methoxy, and C₁-C₂-alkoxycarbonyl, such as methoxycarbonyl.

Particularly preferably, at least one of the heteroatoms of the 6-membered heteroaromatic radical Het and/or one substituent L is located in the ortho-position to the point of attachment of Het to the triazolopyrimidine moiety. Preferred substituents L in the ortho-position are fluorine, chlorine, bromine, iodine, C₁-C₂-alkyl, such as methyl or ethyl, C₁-C₂-fluoroalkyl, such as trifluoromethyl, and C₁-C₂-alkoxy, such as methoxy, furthermore CN, methylthio, methylsulfinyl, methylsulfonyl, nitro or methoxymethyl. Particularly preferred are chlorine, bromine, iodine, CN, C₁-C₂-alkyl, such as methyl or ethyl, C₁-C₂-alkoxy, such as methoxy, methylthio, methylsulfinyl, methylsulfonyl, nitro and methoxymethyl, especially chlorine, bromine or iodine and very especially chlorine. Very particularly preferred substituents L in the ortho position also include CN, methyl, methoxy and methylthio.

A preferred embodiment of the invention relates to compounds of the formula I in which Het is pyridinyl which optionally has 1, 2 or 3 substituents L.

Among these, preference is given to compounds of the formula I in which Het is 2-pyridinyl which has 1 or 2 substituents L. Among these, particular preference is given to compounds in which one of the substituents L is located in the 5-position of the pyridinyl ring, i.e. para to the bond position. Particularly preferred among these compounds are furthermore compounds I in which one of the substituents L is located in the 3-position of the pyridinyl ring, i.e. ortho to the bond position. From among these, preference is furthermore given to compounds of the formula I in which the 2-pyridinyl radical carries a substituent L in the 3-position and a further substituent in the 5-position. Here, L has in particular the meanings mentioned as being preferred. The radical in the 3-position is in particular selected from the group consisting of chlorine, bromine, iodine, C₁-C₂-alkyl, such as methyl or ethyl, C₁-C₂-alkoxy, such as methoxy, methylthio, methylsulfinyl, methylsulfonyl, nitro and methoxymethyl, and is especially chlorine. The radical in the 5-position is selected in particular from the group consisting of fluorine, chlorine, bromine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl, and C(S)NH₂. Preference is given in particular to compounds of the formula I in which Het is one of the radicals below of the formula Het-1, Het-2 or Het-3,

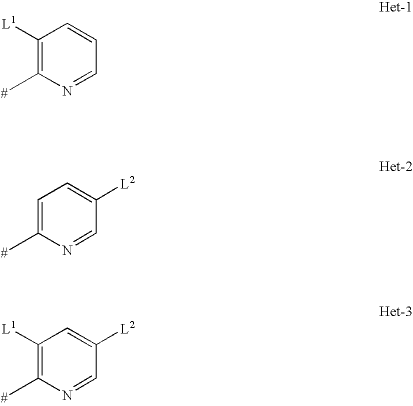

in which # is the point of attachment to the triazolopyrimidine unit and
L¹ is chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl, especially chlorine, and
L² is fluorine, chlorine, bromine, iodine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl, or C(S)NH₂.

Preference is furthermore given to compounds of the formula I in which Het is 3-pyridinyl which has optionally 1, 2 or 3 substituents L. From among these, preference is given to those compounds which have a substituent L in the 2-position (ortho to the point of attachment and to the nitrogen of the pyridine ring) and/or a substituent L in the 4-position of the pyridine ring (ortho to the point of attachment and para to the nitrogen of the pyridine ring). Preference is given in particular to compounds of the formula I in which Het is one of the radicals below of the formula Het-4, Het-5, Het-6, Het-7 or Het-8

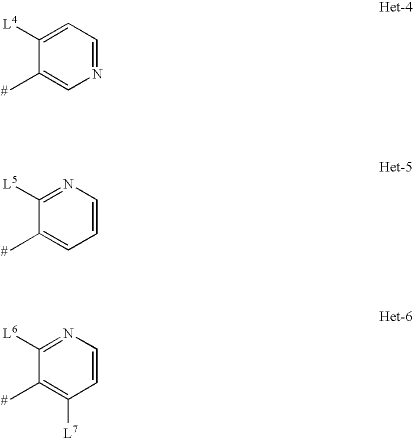

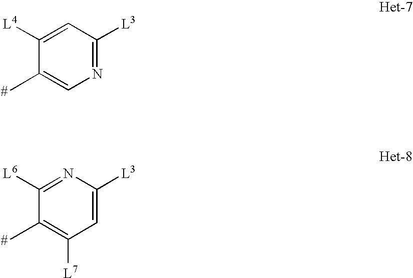

in which # is the point of attachment to the triazolopyrimidine unit and
L³ is fluorine, chlorine, bromine, iodine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl, or C(S)NH₂,
L⁴ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl,
L⁵ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl, and
L⁶ and L⁷ independently of one another have one of the following meanings: fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl.

Preference is furthermore given to compounds of the formula I in which Het is 4-pyridinyl which optionally has 1 or 2 substituents L. Among these, preference is given to those compounds which have a substituent L in the 3-position and/or a substituent L in the 5-position of the pyridine ring. Preference is given in particular to compounds of the formula I in which Het is one of the radicals below of the formula Het-9 or Het-10

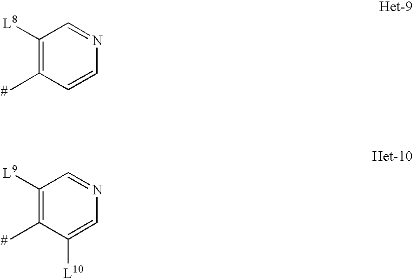

in which # is the point of attachment to the triazolopyrimidine unit and
L⁸ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl,
L⁹ is fluorine, chlorine, bromine, iodine, C₁-C₂-alkyl, such as methyl or ethyl, C₁-C₂-alkoxy, such as methoxy, methylthio, methylsulfinyl, methylsulfonyl, nitro or methoxymethyl and
L¹⁰ represents fluorine, chlorine, bromine, iodine, cyano, nitro, C₁-C₂-alkyl, such as methyl or ethyl, C₁-C₂-alkoxy, such as methoxy, C₁-C₂-alkoxycarbonyl, such as methoxycarbonyl or ethoxycarbonyl, CONH₂, C₁-C₂-alkylaminocarbonyl, such as methylaminocarbonyl or ethylaminocarbonyl, C₁-C₂-alkylcarbonyl, such as acetyl, or C(S)NH₂.

A further preferred embodiment of the invention relates to compounds of the formula I in which Het is 2-pyrazinyl which optionally has 1, 2 or 3 substituents L.

A further preferred embodiment of the invention relates to compounds of the formula I in which Het is 3-pyridazinyl which optionally has 1, 2 or 3 substituents L.

A further preferred embodiment of the invention relates to compounds of the formula I in which Het is 1,3,5-triazinyl which optionally has 1 or 2 substituents L.

Besides, R⁵ and R⁶ independently of one another are preferably hydrogen or C₁-C₄-alkyl.

R⁷ is preferably hydrogen or, in particular, C₁-C₆-alkyl.

R⁸ and R⁹ independently of one another are preferably hydrogen or C₁-C₆-alkyl.

R¹⁰, R¹¹, R¹² and R¹³ independently of one another are preferably selected from the group consisting of hydrogen and C₁-C₆-alkyl.

Furthermore, A¹ is preferably hydrogen, C₁-C₈-alkyl or amino. The index n is preferably 0, 1 or 2.

A² is preferably C₁-C₄-alkoxy, NH₂, C₁-C₄-alkylamino or di-C₁-C₄-alkylamino.

Examples of preferred compounds of the formula I are the compounds I compiled in tables 1 to 11430 below. The groups mentioned in tables 1 to 1430 for a substituent Het are furthermore per se, independently of the combination in which they are mentioned, a particularly preferred embodiment of the substituent in question.

Very particularly preferred compounds of the formula I are those in which Het is 3-chloropyridin-2-yl, 3,5-dichloropyridin-2-yl or 2,4-dichloro-6-methylpyridin-3-yl.

### Table 1

Compounds of the formula I, in which X is chlorine, Y is hydrogen and Het is 2-pyridinyl and the combination of R¹ and R² for each compound corresponds to one row of table A.

### Table 2

Compounds of the formula I, in which X is chlorine, Y is hydrogen and Het is 5-nitropyridin-2-yl and the combination of R¹ and R² for each compound corresponds to one row of table A.

### Table 3

Compounds of the formula I, in which X is chlorine, Y is hydrogen and Het is 5-cyanopyridin-2-yl and the combination of R¹ and R² for each compound corresponds to one row of table A.

### Table 4

Compounds of the formula I, in which X is chlorine, Y is hydrogen and Het is 5-methoxycarbonylpyridin-2-yl and the combination of R¹ and R² for each compound corresponds to one row of table A.

### Table 5

Compounds of the formula I, in which X is chlorine, Y is hydrogen and Het is 5-methylpyridin-2-yl and the combination of R¹ and R² for each compound corresponds to one row of table A.

### Table 6

Compounds of the formula I, in which X is chlorine, Y is hydrogen and Het is 4-methylpyridin-2-yl and the combination of R¹ and R² for each compound corresponds to one row of table A.

### Table 7

Compounds of the formula I, in which X is chlorine, Y is hydrogen and Het is 3-methylpyridin-2-yl and the combination of R¹ and R² for each compound corresponds to one row of table A.

### Table 8

Compounds of the formula I, in which X is chlorine, Y is hydrogen and Het is 3-ethylpyridin-2-yl and the combination of R¹ and R² for each compound corresponds to one row of table A.

### Table 9

Compounds of the formula I, in which X is chlorine, Y is hydrogen and Het is 6-methylpyridin-2-yl and the combination of R¹ and R² for each compound corresponds to one row of table A.

### Table 10

Compounds of the formula I, in which X is chlorine, Y is hydrogen and Het is 5-trifluoromethylpyridin-2-yl and the combination of R¹ and R² for each compound corresponds to one row of table A.

### Table 11

Compounds of the formula I, in which X is chlorine, Y is hydrogen and Het is 3-trifluoromethylpyridin-2-yl and the combination of R¹ and R² for each compound corresponds to one row of table A.

### Table 12

Compounds of the formula I, in which X is chlorine, Y is hydrogen and Het is 5-fluoropyridin-2-yl and the combination of R¹ and R² for each compound corresponds to one row of table A.

### Table 13

Compounds of the formula I, in which X is chlorine, Y is hydrogen and Het is 3-fluoropyridin-2-yl and the combination of R¹ and R² for each compound corresponds to one row of table A.

### Table 14

Compounds of the formula I, in which X is chlorine, Y is hydrogen and Het is 3,5-difluoropyridin-2-yl and the combination of R¹ and R² for each compound corresponds to one row of table A.

### Table 15

Compounds of the formula I, in which X is chlorine, Y is hydrogen and Het is 3,5-dichloropyridin-2-yl and the combination of R¹ and R² for each compound corresponds to one row of table A.

### Table 16

Compounds of the formula I, in which X is chlorine, Y is hydrogen and Het is pyridin-3-yl and the combination of R¹ and R² for each compound corresponds to one row of table A.

### Table 17

Compounds of the formula I, in which X is chlorine, Y is hydrogen and Het is pyridin-4-yl and the combination of R¹ and R² for each compound corresponds to one row of table A.

### Table 18

Compounds of the formula I, in which X is chlorine, Y is hydrogen and Het is pyrazin-2-yl and the combination of R¹ and R² for each compound corresponds to one row of table A.

### Table 19

Compounds of the formula I, in which X is chlorine, Y is hydrogen and Het is pyridazin-3-yl and the combination of R¹ and R² for each compound corresponds to one row of table A.

### Table 20

Compounds of the formula I, in which X is chlorine, Y is hydrogen and Het is 6-chloropyridazin-3-yl and the combination of R¹ and R² for each compound corresponds to one row of table A.

### Table 21

Compounds of the formula I, in which X is chlorine, Y is hydrogen and Het is 6-methoxypyridazin-3-yl and the combination of R¹ and R² for each compound corresponds to one row of table A.

### Table 22

Compounds of the formula I, in which X is chlorine, Y is hydrogen and Het is 1,3,5-triazin-2-yl and the combination of R¹ and R² for each compound corresponds to one row of table A.

### Table 23

Compounds of the formula I, in which X is chlorine, Y is hydrogen and Het is 3-chloropyridin-2-yl and the combination of R¹ and R² for each compound corresponds to one row of table A.

### Table 24

Compounds of the formula I, in which X is chlorine, Y is hydrogen and Het is 3-bromopyridin-2-yl and the combination of R¹ and R² for each compound corresponds to one row of table A.

### Table 25

Compounds of the formula I, in which X is chlorine, Y is hydrogen and Het is 3-iodopyridin-2-yl and the combination of R¹ and R² for each compound corresponds to one row of table A.

### Table 26

Compounds of the formula I, in which X is chlorine, Y is hydrogen and Het is 3-methoxypyridin-2-yl and the combination of R¹ and R² for each compound corresponds to one row of table A.

### Table 27

Compounds of the formula I, in which X is chlorine, Y is hydrogen and Het is 3-methylsulfanylpyridin-2-yl and the combination of R¹ and R² for each compound corresponds to one row of table A.

### Table 28

Compounds of the formula I, in which X is chlorine, Y is hydrogen and Het is 3-methylsulfinylpyridin-2-yl and the combination of R¹ and R² for each compound corresponds to one row of table A.

### Table 29

Compounds of the formula I, in which X is chlorine, Y is hydrogen and Het is 3-methylsulfonylpyridin-2-yl and the combination of R¹ and R² for each compound corresponds to one row of table A.

### Table 30

Compounds of the formula I, in which X is chlorine, Y is hydrogen and Het is 3-nitropyridin-2-yl and the combination of R¹ and R² for each compound corresponds to one row of table A.

### Table 31

Compounds of the formula I, in which X is chlorine, Y is hydrogen and Het is 3-methoxymethylpyridin-2-yl and the combination of R¹ and R² for each compound corresponds to one row of table A.

### Table 32

Compounds of the formula I, in which X is chlorine, Y is hydrogen and Het is 5-chloropyridin-2-yl and the combination of R¹ and R² for each compound corresponds to one row of table A.

### Table 33

Compounds of the formula I, in which X is chlorine, Y is hydrogen and Het is 5-bromopyridin-2-yl and the combination of R¹ and R² for each compound corresponds to one row of table A.

### Table 34

Compounds of the formula I, in which X is chlorine, Y is hydrogen and Het is 5-methoxypyridin-2-yl and the combination of R¹ and R² for each compound corresponds to one row of table A.

### Table 35

Compounds of the formula I, in which X is chlorine, Y is hydrogen and Het is 5-methylsulfanylpyridin-2-yl and the combination of R¹ and R² for each compound corresponds to one row of table A.

### Table 36

Compounds of the formula I, in which X is chlorine, Y is hydrogen and Het is 5-methylsulfinylpyridin-2-yl and the combination of R¹ and R² for each compound corresponds to one row of table A.

### Table 37

Compounds of the formula I, in which X is chlorine, Y is hydrogen and Het is 5-methylsulfonylpyridin-2-yl and the combination of R¹ and R² for each compound corresponds to one row of table A.

### Table 38

Compounds of the formula I, in which X is chlorine, Y is hydrogen and Het is 5-methoxymethylpyridin-2-yl and the combination of R¹ and R² for each compound corresponds to one row of table A.

### Table 39

Compounds of the formula I, in which X is chlorine, Y is hydrogen and Het is 5-ethylpyridin-2-yl and the combination of R¹ and R² for each compound corresponds to one row of table A.

### Table 40

Compounds of the formula I, in which X is chlorine, Y is hydrogen and Het is 5-ethoxycarbonylpyridin-2-yl and the combination of R¹ and R² for each compound corresponds to one row of table A.

### Table 41

Compounds of the formula I, in which X is chlorine, Y is hydrogen and Het is 5-carbamoylpyridin-2-yl and the combination of R¹ and R² for each compound corresponds to one row of table A.

### Table 42

Compounds of the formula I, in which X is chlorine, Y is hydrogen and Het is 5-methylcarbamoylpyridin-2-yl and the combination of R¹ and R² for each compound corresponds to one row of table A.

### Table 43

Compounds of the formula I, in which X is chlorine, Y is hydrogen and Het is 5-dimethylcarbamoylpyridin-2-yl and the combination of R¹ and R² for each compound corresponds to one row of table A.

### Table 44

Compounds of the formula I, in which X is chlorine, Y is hydrogen and Het is 5-thiocarbamoylpyridin-2-yl and the combination of R¹ and R² for each compound corresponds to one row of table A.

### Table 45

Compounds of the formula I, in which X is chlorine, Y is hydrogen and Het is 5-acetylpyridin-2-yl and the combination of R¹ and R² for each compound corresponds to one row of table A.

### Table 46

Compounds of the formula I, in which X is chlorine, Y is hydrogen and Het is 3-fluoro-5-chloropyridin-2-yl and the combination of R¹ and R² for each compound corresponds to one row of table A.

### Table 47

Compounds of the formula I, in which X is chlorine, Y is hydrogen and Het is 3-fluoro-5-bromopyridin-2-yl and the combination of R¹ and R² for each compound corresponds to one row of table A.

### Table 48

Compounds of the formula I, in which X is chlorine, Y is hydrogen and Het is 3-fluoro-5-methylpyridin-2-yl and the combination of R¹ and R² for each compound corresponds to one row of table A.

### Table 49

Compounds of the formula I, in which X is chlorine, Y is hydrogen and Het is 3-fluoro-5-ethylpyridin-2-yl and the combination of R¹ and R² for each compound corresponds to one row of table A.

### Table 50

Compounds of the formula I, in which X is chlorine, Y is hydrogen and Het is 3-fluoro-5-methoxypyridin-2-yl and the combination of R¹ and R² for each compound corresponds to one row of table A.

### Table 51

Compounds of the formula I, in which X is chlorine, Y is hydrogen and Het is 3-fluoro-5-cyanopyridin-2-yl and the combination of R¹ and R² for each compound corresponds to one row of table A.

### Table 52

Compounds of the formula I, in which X is chlorine, Y is hydrogen and Het is 3-fluoro-5-methoxycarbonylpyridin-2-yl and the combination of R¹ and R² for each compound corresponds to one row of table A.

### Table 53

Compounds of the formula I, in which X is chlorine, Y is hydrogen and Het is 3-fluoro-5-ethoxycarbonylpyridin-2-yl and the combination of R¹ and R² for each compound corresponds to one row of table A.

### Table 54

Compounds of the formula I, in which X is chlorine, Y is hydrogen and Het is 3-fluoro-5-carbamoylpyridin-2-yl and the combination of R¹ and R² for each compound corresponds to one row of table A.

### Table 55

Compounds of the formula I, in which X is chlorine, Y is hydrogen and Het is 3-fluoro-5-methylcarbamoylpyridin-2-yl and the combination of R¹ and R² for each compound corresponds to one row of table A.

### Table 56

Compounds of the formula I, in which X is chlorine, Y is hydrogen and Het is 3-fluoro-5-dimethylcarbamoylpyridin-2-yl and the combination of R¹ and R² for each compound corresponds to one row of table A.

### Table 57

Compounds of the formula I, in which X is chlorine, Y is hydrogen and Het is 3-fluoro-5-thiocarbamoylpyridin-2-yl and the combination of R¹ and R² for each compound corresponds to one row of table A.

### Table 58

Compounds of the formula I, in which X is chlorine, Y is hydrogen and Het is 3-fluoro-5-nitropyridin-2-yl and the combination of R¹ and R² for each compound corresponds to one row of table A.

### Table 59

Compounds of the formula I, in which X is chlorine, Y is hydrogen and Het is 3-fluoro-5-acetylpyridin-2-yl and the combination of R¹ and R² for each compound corresponds to one row of table A.

### Table 60

Compounds of the formula I, in which X is chlorine, Y is hydrogen and Het is 3-chloro-5-fluoropyridin-2-yl and the combination of R¹ and R² for each compound corresponds to one row of table A.

### Table 61

Compounds of the formula I, in which X is chlorine, Y is hydrogen and Het is 3-chloro-5-bromopyridin-2-yl and the combination of R¹ and R² for each compound corresponds to one row of table A.

### Table 62

Compounds of the formula I, in which X is chlorine, Y is hydrogen and Het is 3-chloro-5-methylpyridin-2-yl and the combination of R¹ and R² for each compound corresponds to one row of table A.

### Table 63

Compounds of the formula I, in which X is chlorine, Y is hydrogen and Het is 3-chloro-5-ethylpyridin-2-yl and the combination of R¹ and R² for each compound corresponds to one row of table A.

### Table 64

Compounds of the formula I, in which X is chlorine, Y is hydrogen and Het is 3-chloro-5-methoxypyridin-2-yl and the combination of R¹ and R² for each compound corresponds to one row of table A.

### Table 65

Compounds of the formula I, in which X is chlorine, Y is hydrogen and Het is 3-chloro-5-cyanopyridin-2-yl and the combination of R¹ and R² for each compound corresponds to one row of table A.

### Table 66

Compounds of the formula I, in which X is chlorine, Y is hydrogen and Het is 3-chloro-5-methoxycarbonylpyridin-2-yl and the combination of R¹ and R² for each compound corresponds to one row of table A.

### Table 67

Compounds of the formula I, in which X is chlorine, Y is hydrogen and Het is 3-chloro-5-ethoxycarbonylpyridin-2-yl and the combination of R¹ and R² for each compound corresponds to one row of table A.

### Table 68

Compounds of the formula I, in which X is chlorine, Y is hydrogen and Het is 3-chloro-5-carbamoylpyridin-2-yl and the combination of R¹ and R² for each compound corresponds to one row of table A.

### Table 69

Compounds of the formula I, in which X is chlorine, Y is hydrogen and Het is 3-chloro-5-methylcarbamoylpyridin-2-yl and the combination of R¹ and R² for each compound corresponds to one row of table A.

### Table 70

Compounds of the formula I, in which X is chlorine, Y is hydrogen and Het is 3-chloro-5-dimethylcarbamoylpyridin-2-yl and the combination of R¹ and R² for each compound corresponds to one row of table A.

### Table 71

Compounds of the formula I, in which X is chlorine, Y is hydrogen and Het is 3-chloro-5-thiocarbamoylpyridin-2-yl and the combination of R¹ and R² for each compound corresponds to one row of table A.

### Table 72

Compounds of the formula I, in which X is chlorine, Y is hydrogen and Het is 3-chloro-5-nitropyridin-2-yl and the combination of R¹ and R² for each compound corresponds to one row of table A.

### Table 73

Compounds of the formula I, in which X is chlorine, Y is hydrogen and Het is 3-chloro-5-acetylpyridin-2-yl and the combination of R¹ and R² for each compound corresponds to one row of table A.

### Table 74

Compounds of the formula I, in which X is chlorine, Y is hydrogen and Het is 3-methyl-5-fluoropyridin-2-yl and the combination of R¹ and R² for each compound corresponds to one row of table A.

### Table 75

Compounds of the formula I, in which X is chlorine, Y is hydrogen and Het is 3-methyl-5-chloropyridin-2-yl and the combination of R¹ and R² for each compound corresponds to one row of table A.

### Table 76

Compounds of the formula I, in which X is chlorine, Y is hydrogen and Het is 3-methyl-5-bromopyridin-2-yl and the combination of R¹ and R² for each compound corresponds to one row of table A.

### Table 77

Compounds of the formula I, in which X is chlorine, Y is hydrogen and Het is 3,5-dimethylpyridin-2-yl and the combination of R¹ and R² for each compound corresponds to one row of table A.

### Table 78

Compounds of the formula I, in which X is chlorine, Y is hydrogen and Het is 3-methyl-5-ethylpyridin-2-yl and the combination of R¹ and R² for each compound corresponds to one row of table A.

### Table 79

Compounds of the formula I, in which X is chlorine, Y is hydrogen and Het is 3-methyl-5-methoxypyridin-2-yl and the combination of R¹ and R² for each compound corresponds to one row of table A.

### Table 80

Compounds of the formula I, in which X is chlorine, Y is hydrogen and Het is 3-methyl-5-cyanopyridin-2-yl and the combination of R¹ and R² for each compound corresponds to one row of table A.

### Table 81

Compounds of the formula I, in which X is chlorine, Y is hydrogen and Het is 3-methyl-5-methoxycarbonylpyridin-2-yl and the combination of R¹ and R² for each compound corresponds to one row of table A.

### Table 82

Compounds of the formula I, in which X is chlorine, Y is hydrogen and Het is 3-methyl-5-ethoxycarbonylpyridin-2-yl and the combination of R¹ and R² for each compound corresponds to one row of table A.

### Table 83

Compounds of the formula I, in which X is chlorine, Y is hydrogen and Het is 3-methyl-5-carbamoylpyridin-2-yl and the combination of R¹ and R² for each compound corresponds to one row of table A.

### Table 84

Compounds of the formula I, in which X is chlorine, Y is hydrogen and Het is 3-methyl-5-methylcarbamoylpyridin-2-yl and the combination of R¹ and R² for each compound corresponds to one row of table A.

### Table 85

Compounds of the formula I, in which X is chlorine, Y is hydrogen and Het is 3-methyl-5-dimethylcarbamoylpyridin-2-yl and the combination of R¹ and R² for each compound corresponds to one row of table A.

### Table 86

Compounds of the formula I, in which X is chlorine, Y is hydrogen and Het is 3-methyl-5-thiocarbamoylpyridin-2-yl and the combination of R¹ and R² for each compound corresponds to one row of table A.

### Table 87

Compounds of the formula I, in which X is chlorine, Y is hydrogen and Het is 3-methyl-5-nitropyridin-2-yl and the combination of R¹ and R² for each compound corresponds to one row of table A.

### Table 88

Compounds of the formula I, in which X is chlorine, Y is hydrogen and Het is 3-methyl-5-acetylpyridin-2-yl and the combination of R¹ and R² for each compound corresponds to one row of table A.

### Table 89

Compounds of the formula I, in which X is chlorine, Y is hydrogen and Het is 3-bromo-5-fluoropyridin-2-yl and the combination of R¹ and R² for each compound corresponds to one row of table A.

### Table 90

Compounds of the formula I, in which X is chlorine, Y is hydrogen and Het is 3-bromo-5-chloropyridin-2-yl and the combination of R¹ and R² for each compound corresponds to one row of table A.

### Table 91

Compounds of the formula I, in which X is chlorine, Y is hydrogen and Het is 3-bromo-5-methylpyridin-2-yl and the combination of R¹ and R² for each compound corresponds to one row of table A.

### Table 92

Compounds of the formula I, in which X is chlorine, Y is hydrogen and Het is 3-methylsulfonyl-5-fluoropyridin-2-yl and the combination of R¹ and R² for each compound corresponds to one row of table A.

### Table 93

Compounds of the formula I, in which X is chlorine, Y is hydrogen and Het is 3-methylsulfonyl-5-chloropyridin-2-yl and the combination of R¹ and R² for each compound corresponds to one row of table A.

### Table 94

Compounds of the formula I, in which X is chlorine, Y is hydrogen and Het is 3-methylsulfonyl-5-methylpyridin-2-yl and the combination of R¹ and R² for each compound corresponds to one row of table A.

### Table 95

Compounds of the formula I, in which X is chlorine, Y is hydrogen and Het is 3-methoxy-5-fluoropyridin-2-yl and the combination of R¹ and R² for each compound corresponds to one row of table A.

### Table 96

Compounds of the formula I, in which X is chlorine, Y is hydrogen and Het is 3-methoxy-5-chloropyridin-2-yl and the combination of R¹ and R² for each compound corresponds to one row of table A.

### Table 97

Compounds of the formula I, in which X is chlorine, Y is hydrogen and Het is 3-methoxy-5-methylpyridin-2-yl and the combination of R¹ and R² for each compound corresponds to one row of table A.

### Table 98

Compounds of the formula I, in which X is chlorine, Y is hydrogen and Het is 3-methylsulfanyl-5-fluoropyridin-2-yl and the combination of R¹ and R² for each compound corresponds to one row of table A.

### Table 99

Compounds of the formula I, in which X is chlorine, Y is hydrogen and Het is 3-methylsulfanyl-5-chloropyridin-2-yl and the combination of R¹ and R² for each compound corresponds to one row of table A.

### Table 100

Compounds of the formula I, in which X is chlorine, Y is hydrogen and Het is 3-methylsulfanyl-5-methylpyridin-2-yl and the combination of R¹ and R² for each compound corresponds to one row of table A.

### Table 101

Compounds of the formula I, in which X is chlorine, Y is hydrogen and Het is 3-nitro-5-fluoropyridin-2-yl and the combination of R¹ and R² for each compound corresponds to one row of table A.

### Table 102

Compounds of the formula I, in which X is chlorine, Y is hydrogen and Het is 3-nitro-5-chloropyridin-2-yl and the combination of R¹ and R² for each compound corresponds to one row of table A.

### Table 103

Compounds of the formula I, in which X is chlorine, Y is hydrogen and Het is 3-nitro-5-methylpyridin-2-yl and the combination of R¹ and R² for each compound corresponds to one row of table A.

### Table 104

Compounds of the formula I, in which X is chlorine, Y is hydrogen and Het is 3,5-dibromopyridin-2-yl and the combination of R¹ and R² for each compound corresponds to one row of table A.

### Table 105

Compounds of the formula I, in which X is chlorine, Y is hydrogen and Het is 3-fluoropyridin-4-yl and the combination of R¹ and R² for each compound corresponds to one row of table A.

### Table 106

Compounds of the formula I, in which X is chlorine, Y is hydrogen and Het is 3-chloropyridin-4-yl and the combination of R¹ and R² for each compound corresponds to one row of table A.

### Table 107

Compounds of the formula I, in which X is chlorine, Y is hydrogen and Het is 3-bromopyridin-4-yl and the combination of R¹ and R² for each compound corresponds to one row of table A.

### Table 108

Compounds of the formula I, in which X is chlorine, Y is hydrogen and Het is 3-methylpyridin-4-yl and the combination of R¹ and R² for each compound corresponds to one row of table A.

### Table 109

Compounds of the formula I, in which X is chlorine, Y is hydrogen and Het is 3-methoxypyridin-4-yl and the combination of R¹ and R² for each compound corresponds to one row of table A.

### Table 110

Compounds of the formula I, in which X is chlorine, Y is hydrogen and Het is 3-methylsulfanylpyridin-4-yl and the combination of R¹ and R² for each compound corresponds to one row of table A.

### Table 111

Compounds of the formula I, in which X is chlorine, Y is hydrogen and Het is 3-methylsulfinylpyridin-4-yl and the combination of R¹ and R² for each compound corresponds to one row of table A.

### Table 112

Compounds of the formula I, in which X is chlorine, Y is hydrogen and Het is 3-methylsulfonylpyridin-4-yl and the combination of R¹ and R² for each compound corresponds to one row of table A.

### Table 113

Compounds of the formula I, in which X is chlorine, Y is hydrogen and Het is 3-nitropyridin-4-yl and the combination of R¹ and R² for each compound corresponds to one row of table A.

### Table 114

Compounds of the formula I, in which X is chlorine, Y is hydrogen and Het is 3-methoxymethylpyridin-4-yl and the combination of R¹ and R² for each compound corresponds to one row of table A.

### Table 115

Compounds of the formula I, in which X is chlorine, Y is hydrogen and Het is 3,5-difluoropyridin-4-yl and the combination of R¹ and R² for each compound corresponds to one row of table A.

### Table 116

Compounds of the formula I, in which X is chlorine, Y is hydrogen and Het is 3-fluoro-5-chloropyridin-4-yl and the combination of R¹ and R² for each compound corresponds to one row of table A.

### Table 117

Compounds of the formula I, in which X is chlorine, Y is hydrogen and Het is 3-fluoro-5-bromopyridin-4-yl and the combination of R¹ and R² for each compound corresponds to one row of table A.

### Table 118

Compounds of the formula I, in which X is chlorine, Y is hydrogen and Het is 3-fluoro-5-methylpyridin-4-yl and the combination of R¹ and R² for each compound corresponds to one row of table A.

### Table 119

Compounds of the formula I, in which X is chlorine, Y is hydrogen and Het is 3-fluoro-5-ethylpyridin-4-yl and the combination of R¹ and R² for each compound corresponds to one row of table A.

### Table 120

Compounds of the formula I, in which X is chlorine, Y is hydrogen and Het is 3-fluoro-5-methoxypyridin-4-yl and the combination of R¹ and R² for each compound corresponds to one row of table A.

### Table 121

Compounds of the formula I, in which X is chlorine, Y is hydrogen and Het is 3-fluoro-5-cyanopyridin-4-yl and the combination of R¹ and R² for each compound corresponds to one row of table A.

### Table 122

Compounds of the formula I, in which X is chlorine, Y is hydrogen and Het is 3-fluoro-5-methoxycarbonylpyridin-4-yl and the combination of R¹ and R² for each compound corresponds to one row of table A.

### Table 123

Compounds of the formula I, in which X is chlorine, Y is hydrogen and Het is 3-fluoro-5-ethoxycarbonylpyridin-4-yl and the combination of R¹ and R² for each compound corresponds to one row of table A.

### Table 124

Compounds of the formula I, in which X is chlorine, Y is hydrogen and Het is 3-fluoro-5-carbamoylpyridin-4-yl and the combination of R¹ and R² for each compound corresponds to one row of table A.

### Table 125

Compounds of the formula I, in which X is chlorine, Y is hydrogen and Het is 3-fluoro-5-methylcarbamoylpyridin-4-yl and the combination of R¹ and R² for each compound corresponds to one row of table A.

### Table 126

Compounds of the formula I, in which X is chlorine, Y is hydrogen and Het is 3-fluoro-5-dimethylcarbamoylpyridin-4-yl and the combination of R¹ and R² for each compound corresponds to one row of table A.

### Table 127

Compounds of the formula I, in which X is chlorine, Y is hydrogen and Het is 3-fluoro-5-thiocarbamoylpyridin-4-yl and the combination of R¹ and R² for each compound corresponds to one row of table A.

### Table 128

Compounds of the formula I, in which X is chlorine, Y is hydrogen and Het is 3-fluoro-5-nitropyridin-4-yl and the combination of R¹ and R² for each compound corresponds to one row of table A.

### Table 129

Compounds of the formula I, in which X is chlorine, Y is hydrogen and Het is 3-fluoro-5-acetylpyridin-4-yl and the combination of R¹ and R² for each compound corresponds to one row of table A.

### Table 130

Compounds of the formula I, in which X is chlorine, Y is hydrogen and Het is 3,5-dichloro-5-chloropyridin-4-yl and the combination of R¹ and R² for each compound corresponds to one row of table A.

### Table 131

Compounds of the formula I, in which X is chlorine, Y is hydrogen and Het is 3-chloro-5-bromopyridin-4-yl and the combination of R¹ and R² for each compound corresponds to one row of table A.

### Table 132

Compounds of the formula I, in which X is chlorine, Y is hydrogen and Het is 3-chloro-5-methylpyridin-4-yl and the combination of R¹ and R² for each compound corresponds to one row of table A.

### Table 133

Compounds of the formula I, in which X is chlorine, Y is hydrogen and Het is 3-chloro-5-ethylpyridin-4-yl and the combination of R¹ and R² for each compound corresponds to one row of table A.

### Table 134

Compounds of the formula I, in which X is chlorine, Y is hydrogen and Het is 3-chloro-5-methoxypyridin-4-yl and the combination of R¹ and R² for each compound corresponds to one row of table A.

### Table 135

Compounds of the formula I, in which X is chlorine, Y is hydrogen and Het is 3-chloro-5-cyanopyridin-4-yl and the combination of R¹ and R² for each compound corresponds to one row of table A.

### Table 136

Compounds of the formula I, in which X is chlorine, Y is hydrogen and Het is 3-chloro-5-methoxycarbonylpyridin-4-yl and the combination of R¹ and R² for each compound corresponds to one row of table A.

### Table 137

Compounds of the formula I, in which X is chlorine, Y is hydrogen and Het is 3-chloro-5-ethoxycarbonylpyridin-4-yl and the combination of R¹ and R² for each compound corresponds to one row of table A.

### Table 138

Compounds of the formula I, in which X is chlorine, Y is hydrogen and Het is 3-chloro-5-carbamoylpyridin-4-yl and the combination of R¹ and R² for each compound corresponds to one row of table A.

### Table 139

Compounds of the formula I, in which X is chlorine, Y is hydrogen and Het is 3-chloro-5-methylcarbamoylpyridin-4-yl and the combination of R¹ and R² for each compound corresponds to one row of table A.

### Table 140

Compounds of the formula I, in which X is chlorine, Y is hydrogen and Het is 3-chloro-5-dimethylcarbamoylpyridin-4-yl and the combination of R¹ and R² for each compound corresponds to one row of table A.

### Table 141

Compounds of the formula I, in which X is chlorine, Y is hydrogen and Het is 3-chloro-5-thiocarbamoylpyridin-4-yl and the combination of R¹ and R² for each compound corresponds to one row of table A.

### Table 142

Compounds of the formula I, in which X is chlorine, Y is hydrogen and Het is 3-chloro-5-nitropyridin-4-yl and the combination of R¹ and R² for each compound corresponds to one row of table A.

### Table 143

Compounds of the formula I, in which X is chlorine, Y is hydrogen and Het is 3-chloro-5-acetylpyridin-4-yl and the combination of R¹ and R² for each compound corresponds to one row of table A.

### Table 144

Compounds of the formula I, in which X is chlorine, Y is hydrogen and Het is 3,5-dimethylpyridin-4-yl and the combination of R¹ and R² for each compound corresponds to one row of table A.

### Table 145

Compounds of the formula I, in which X is chlorine, Y is hydrogen and Het is 3-methyl-5-bromopyridin-4-yl and the combination of R¹ and R² for each compound corresponds to one row of table A.

### Table 146

Compounds of the formula I, in which X is chlorine, Y is hydrogen and Het is 3-methyl-5-ethylpyridin-4-yl and the combination of R¹ and R² for each compound corresponds to one row of table A.

### Table 147

Compounds of the formula I, in which X is chlorine, Y is hydrogen and Het is 3-methyl-5-methoxypyridin-4-yl and the combination of R¹ and R² for each compound corresponds to one row of table A.

### Table 148

Compounds of the formula I, in which X is chlorine, Y is hydrogen and Het is 3-methyl-5-cyanopyridin-4-yl and the combination of R¹ and R² for each compound corresponds to one row of table A.

### Table 149

Compounds of the formula I, in which X is chlorine, Y is hydrogen and Het is 3-methyl-5-methoxycarbonylpyridin-4-yl and the combination of R¹ and R² for each compound corresponds to one row of table A.

### Table 150

Compounds of the formula I, in which X is chlorine, Y is hydrogen and Het is 3-methyl-5-ethoxycarbonylpyridin-4-yl and the combination of R¹ and R² for each compound corresponds to one row of table A.

### Table 151

Compounds of the formula I, in which X is chlorine, Y is hydrogen and Het is 3-methyl-5-carbamoylpyridin-4-yl and the combination of R¹ and R² for each compound corresponds to one row of table A.

### Table 152

Compounds of the formula I, in which X is chlorine, Y is hydrogen and Het is 3-methyl-5-methylcarbamoylpyridin-4-yl and the combination of R¹ and R² for each compound corresponds to one row of table A.

### Table 153

Compounds of the formula I, in which X is chlorine, Y is hydrogen and Het is 3-methyl-5-dimethylcarbamoylpyridin-4-yl and the combination of R¹ and R² for each compound corresponds to one row of table A.

### Table 154

Compounds of the formula I, in which X is chlorine, Y is hydrogen and Het is 3-methyl-5-thiocarbamoylpyridin-4-yl and the combination of R¹ and R² for each compound corresponds to one row of table A.

### Table 155

Compounds of the formula I, in which X is chlorine, Y is hydrogen and Het is 3-methyl-5-nitropyridin-4-yl and the combination of R¹ and R² for each compound corresponds to one row of table A.

### Table 156

Compounds of the formula I, in which X is chlorine, Y is hydrogen and Het is 3-methyl-5-acetylpyridin-4-yl and the combination of R¹ and R² for each compound corresponds to one row of table A.

### Table 157

Compounds of the formula I, in which X is chlorine, Y is hydrogen and Het is 3-methylsulfanyl-5-fluoropyridin-4-yl and the combination of R¹ and R² for each compound corresponds to one row of table A.

### Table 158

Compounds of the formula I, in which X is chlorine, Y is hydrogen and Het is 3-methylsulfanyl-5-chloropyridin-4-yl and the combination of R¹ and R² for each compound corresponds to one row of table A.

### Table 159

Compounds of the formula I, in which X is chlorine, Y is hydrogen and Het is 3-methylsulfanyl-5-methylpyridin-4-yl and the combination of R¹ and R² for each compound corresponds to one row of table A.

### Table 160

Compounds of the formula I, in which X is chlorine, Y is hydrogen and Het is 3-methylsulfinyl-5-fluoropyridin-4-yl and the combination of R¹ and R² for each compound corresponds to one row of table A.

### Table 161

Compounds of the formula I, in which X is chlorine, Y is hydrogen and Het is 3-methylsulfinyl-5-chloropyridin-4-yl and the combination of R¹ and R² for each compound corresponds to one row of table A.

### Table 162

Compounds of the formula I, in which X is chlorine, Y is hydrogen and Het is 3-methylsulfinyl-5-methylpyridin-4-yl and the combination of R¹ and R² for each compound corresponds to one row of table A.

### Table 163

Compounds of the formula I, in which X is chlorine, Y is hydrogen and Het is 3-methylsulfonyl-5-fluoropyridin-4-yl and the combination of R¹ and R² for each compound corresponds to one row of table A.

### Table 164

Compounds of the formula I, in which X is chlorine, Y is hydrogen and Het is 3-methylsulfonyl-5-chloropyridin-4-yl and the combination of R¹ and R² for each compound corresponds to one row of table A.

### Table 165

Compounds of the formula I, in which X is chlorine, Y is hydrogen and Het is 3-methylsulfonyl-5-methylpyridin-4-yl and the combination of R¹ and R² for each compound corresponds to one row of table A.

### Table 166

Compounds of the formula I, in which X is chlorine, Y is hydrogen and Het is 3-methoxymethyl-5-fluoropyridin-4-yl and the combination of R¹ and R² for each compound corresponds to one row of table A.

### Table 167

Compounds of the formula I, in which X is chlorine, Y is hydrogen and Het is 3-methoxymethyl-5-chloropyridin-4-yl and the combination of R¹ and R² for each compound corresponds to one row of table A.

### Table 168

Compounds of the formula I, in which X is chlorine, Y is hydrogen and Het is 3-methoxymethyl-5-methylpyridin-4-yl and the combination of R¹ and R² for each compound corresponds to one row of table A.

### Table 169

Compounds of the formula I, in which X is chlorine, Y is hydrogen and Het is 2-fluoropyridin-3-yl and the combination of R¹ and R² for each compound corresponds to one row of table A.

### Table 170

Compounds of the formula I, in which X is chlorine, Y is hydrogen and Het is 2-chloropyridin-3-yl and the combination of R¹ and R² for each compound corresponds to one row of table A.

### Table 171

Compounds of the formula I, in which X is chlorine, Y is hydrogen and Het is 2-bromopyridin-3-yl and the combination of R¹ and R² for each compound corresponds to one row of table A.

### Table 172

Compounds of the formula I, in which X is chlorine, Y is hydrogen and Het is 2-methylpyridin-3-yl and the combination of R¹ and R² for each compound corresponds to one row of table A.

### Table 173

Compounds of the formula I, in which X is chlorine, Y is hydrogen and Het is 2-methoxypyridin-3-yl and the combination of R¹ and R² for each compound corresponds to one row of table A.

### Table 174

Compounds of the formula I, in which X is chlorine, Y is hydrogen and Het is 2-methylsulfanylpyridin-3-yl and the combination of R¹ and R² for each compound corresponds to one row of table A.

### Table 175

Compounds of the formula I, in which X is chlorine, Y is hydrogen and Het is 2-methylsulfinylpyridin-3-yl and the combination of R¹ and R² for each compound corresponds to one row of table A.

### Table 176

Compounds of the formula I, in which X is chlorine, Y is hydrogen and Het is 2-methylsulfonylpyridin-3-yl and the combination of R¹ and R² for each compound corresponds to one row of table A.

### Table 177

Compounds of the formula I, in which X is chlorine, Y is hydrogen and Het is 2-nitropyridin-3-yl and the combination of R¹ and R² for each compound corresponds to one row of table A.

### Table 178

Compounds of the formula I, in which X is chlorine, Y is hydrogen and Het is 2-methoxymethylpyridin-3-yl and the combination of R¹ and R² for each compound corresponds to one row of table A.

### Table 179

Compounds of the formula I, in which X is chlorine, Y is hydrogen and Het is 4-fluoropyridin-3-yl and the combination of R¹ and R² for each compound corresponds to one row of table A.

### Table 180

Compounds of the formula I, in which X is chlorine, Y is hydrogen and Het is 4-chloropyridin-3-yl and the combination of R¹ and R² for each compound corresponds to one row of table A.

### Table 181

Compounds of the formula I, in which X is chlorine, Y is hydrogen and Het is 4-bromopyridin-3-yl and the combination of R¹ and R² for each compound corresponds to one row of table A.

### Table 182

Compounds of the formula I, in which X is chlorine, Y is hydrogen and Het is 4-methylpyridin-3-yl and the combination of R¹ and R² for each compound corresponds to one row of table A.

### Table 183

Compounds of the formula I, in which X is chlorine, Y is hydrogen and Het is 4-methoxypyridin-3-yl and the combination of R¹ and R² for each compound corresponds to one row of table A.

### Table 184

Compounds of the formula I, in which X is chlorine, Y is hydrogen and Het is 4-methylsulfanylpyridin-3-yl and the combination of R¹ and R² for each compound corresponds to one row of table A.

### Table 185

Compounds of the formula I, in which X is chlorine, Y is hydrogen and Het is 4-methylsulfinylpyridin-3-yl and the combination of R¹ and R² for each compound corresponds to one row of table A.

### Table 186

Compounds of the formula I, in which X is chlorine, Y is hydrogen and Het is 4-methylsulfonylpyridin-3-yl and the combination of R¹ and R² for each compound corresponds to one row of table A.

### Table 187

Compounds of the formula I, in which X is chlorine, Y is hydrogen and Het is 4-nitropyridin-3-yl and the combination of R¹ and R² for each compound corresponds to one row of table A.

### Table 188

Compounds of the formula I, in which X is chlorine, Y is hydrogen and Het is 4-methoxymethylpyridin-3-yl and the combination of R¹ and R² for each compound corresponds to one row of table A.

### Table 189

Compounds of the formula I, in which X is chlorine, Y is hydrogen and Het is 2,4-difluoropyridin-3-yl and the combination of R¹ and R² for each compound corresponds to one row of table A.

### Table 190

Compounds of the formula I, in which X is chlorine, Y is hydrogen and Het is 2-fluoro-4-chloropyridin-3-yl and the combination of R¹ and R² for each compound corresponds to one row of table A.

### Table 191

Compounds of the formula I, in which X is chlorine, Y is hydrogen and Het is 2-fluoro-4-bromopyridin-3-yl and the combination of R¹ and R² for each compound corresponds to one row of table A.

### Table 192

Compounds of the formula I, in which X is chlorine, Y is hydrogen and Het is 2-fluoro-4-methylpyridin-3-yl and the combination of R¹ and R² for each compound corresponds to one row of table A.

### Table 193

Compounds of the formula I, in which X is chlorine, Y is hydrogen and Het is 2-fluoro-4-methoxypyridin-3-yl and the combination of R¹ and R² for each compound corresponds to one row of table A.

### Table 194

Compounds of the formula I, in which X is chlorine, Y is hydrogen and Het is 2-fluoro-4-methylsulfanylpyridin-3-yl and the combination of R¹ and R² for each compound corresponds to one row of table A.

### Table 195

Compounds of the formula I, in which X is chlorine, Y is hydrogen and Het is 2-fluoro-4-methylsulfinylpyridin-3-yl and the combination of R¹ and R² for each compound corresponds to one row of table A.

### Table 196

Compounds of the formula I, in which X is chlorine, Y is hydrogen and Het is 2-fluoro-4-methylsulfonylpyridin-3-yl and the combination of R¹ and R² for each compound corresponds to one row of table A.

### Table 197

Compounds of the formula I, in which X is chlorine, Y is hydrogen and Het is 2-fluoro-4-nitropyridin-3-yl and the combination of R¹ and R² for each compound corresponds to one row of table A.

### Table 198

Compounds of the formula I, in which X is chlorine, Y is hydrogen and Het is 2-fluoro-4-methoxymethylpyridin-3-yl and the combination of R¹ and R² for each compound corresponds to one row of table A.

### Table 199

Compounds of the formula I, in which X is chlorine, Y is hydrogen and Het is 2,4-dichloropyridin-3-yl and the combination of R¹ and R² for each compound corresponds to one row of table A.

### Table 200

Compounds of the formula I, in which X is chlorine, Y is hydrogen and Het is 2-chloro-4-fluoropyridin-3-yl and the combination of R¹ and R² for each compound corresponds to one row of table A.

### Table 201

Compounds of the formula I, in which X is chlorine, Y is hydrogen and Het is 2-chloro-4-bromopyridin-3-yl and the combination of R¹ and R² for each compound corresponds to one row of table A.

### Table 202

Compounds of the formula I, in which X is chlorine, Y is hydrogen and Het is 2-chloro-4-methylpyridin-3-yl and the combination of R¹ and R² for each compound corresponds to one row of table A.

### Table 203

Compounds of the formula I, in which X is chlorine, Y is hydrogen and Het is 2-chloro-4-methoxypyridin-3-yl and the combination of R¹ and R² for each compound corresponds to one row of table A.

### Table 204

Compounds of the formula I, in which X is chlorine, Y is hydrogen and Het is 2-chloro-4-methylsulfanylpyridin-3-yl and the combination of R¹ and R² for each compound corresponds to one row of table A.

### Table 205

Compounds of the formula I, in which X is chlorine, Y is hydrogen and Het is 2-chloro-4-methylsulfinylpyridin-3-yl and the combination of R¹ and R² for each compound corresponds to one row of table A.

### Table 206

Compounds of the formula I, in which X is chlorine, Y is hydrogen and Het is 2-chloro-4-methylsulfonylpyridin-3-yl and the combination of R¹ and R² for each compound corresponds to one row of table A.

### Table 207

Compounds of the formula I, in which X is chlorine, Y is hydrogen and Het is 2-chloro-4-nitropyridin-3-yl and the combination of R¹ and R² for each compound corresponds to one row of table A.

### Table 208

Compounds of the formula I, in which X is chlorine, Y is hydrogen and Het is 2-chloro-4-methoxymethylpyridin-3-yl and the combination of R¹ and R² for each compound corresponds to one row of table A.

### Table 209

Compounds of the formula I, in which X is chlorine, Y is hydrogen and Het is 2,4-dimethylpyridin-3-yl and the combination of R¹ and R² for each compound corresponds to one row of table A.

### Table 210

Compounds of the formula I, in which X is chlorine, Y is hydrogen and Het is 2-methyl-4-chloropyridin-3-yl and the combination of R¹ and R² for each compound corresponds to one row of table A.

### Table 211

Compounds of the formula I, in which X is chlorine, Y is hydrogen and Het is 2-methyl-4-fluoropyridin-3-yl and the combination of R¹ and R² for each compound corresponds to one row of table A.

### Table 212

Compounds of the formula I, in which X is chlorine, Y is hydrogen and Het is 2-methyl-4-bromopyridin-3-yl and the combination of R¹ and R² for each compound corresponds to one row of table A.

### Table 213

Compounds of the formula I, in which X is chlorine, Y is hydrogen and Het is 2-methyl-4-methoxypyridin-3-yl and the combination of R¹ and R² for each compound corresponds to one row of table A.

### Table 214

Compounds of the formula I, in which X is chlorine, Y is hydrogen and Het is 2-methyl-4-methylsulfanylpyridin-3-yl and the combination of R¹ and R² for each compound corresponds to one row of table A.

### Table 215

Compounds of the formula I, in which X is chlorine, Y is hydrogen and Het is 2-methyl-4-methylsulfinylpyridin-3-yl and the combination of R¹ and R² for each compound corresponds to one row of table A.

### Table 216

Compounds of the formula I, in which X is chlorine, Y is hydrogen and Het is 2-methyl-4-methylsulfonylpyridin-3-yl and the combination of R¹ and R² for each compound corresponds to one row of table A.

### Table 217

Compounds of the formula I, in which X is chlorine, Y is hydrogen and Het is 2-methyl-4-nitropyridin-3-yl and the combination of R¹ and R² for each compound corresponds to one row of table A.

### Table 218

Compounds of the formula I, in which X is chlorine, Y is hydrogen and Het is 2-methyl-4-methoxymethylpyridin-3-yl and the combination of R¹ and R² for each compound corresponds to one row of table A.

### Table 219

Compounds of the formula I, in which X is chlorine, Y is hydrogen and Het is 2-bromo-4-fluoropyridin-3-yl and the combination of R¹ and R² for each compound corresponds to one row of table A.

### Table 220

Compounds of the formula I, in which X is chlorine, Y is hydrogen and Het is 2-bromo-4-chloropyridin-3-yl and the combination of R¹ and R² for each compound corresponds to one row of table A.

### Table 221

Compounds of the formula I, in which X is chlorine, Y is hydrogen and Het is 2-bromo-4-methylpyridin-3-yl and the combination of R¹ and R² for each compound corresponds to one row of table A.

### Table 222

Compounds of the formula I, in which X is chlorine, Y is hydrogen and Het is 2-methoxy-4-fluoropyridin-3-yl and the combination of R¹ and R² for each compound corresponds to one row of table A.

### Table 223

Compounds of the formula I, in which X is chlorine, Y is hydrogen and Het is 2-methoxy-4-chloropyridin-3-yl and the combination of R¹ and R² for each compound corresponds to one row of table A.

### Table 224

Compounds of the formula I, in which X is chlorine, Y is hydrogen and Het is 2-methoxy-4-methylpyridin-3-yl and the combination of R¹ and R² for each compound corresponds to one row of table A.

### Table 225

Compounds of the formula I, in which X is chlorine, Y is hydrogen and Het is 2-methylsulfanyl-4-fluoropyridin-3-yl and the combination of R¹ and R² for each compound corresponds to one row of table A.

### Table 226

Compounds of the formula I, in which X is chlorine, Y is hydrogen and Het is 2-methylsulfanyl-4-chloropyridin-3-yl and the combination of R¹ and R² for each compound corresponds to one row of table A.

### Table 227

Compounds of the formula I, in which X is chlorine, Y is hydrogen and Het is 2-methylsulfanyl-4-methylpyridin-3-yl and the combination of R¹ and R² for each compound corresponds to one row of table A.

### Table 228

Compounds of the formula I, in which X is chlorine, Y is hydrogen and Het is 2-methylsulfinyl-4-fluoropyridin-3-yl and the combination of R¹ and R² for each compound corresponds to one row of table A.

### Table 229

Compounds of the formula I, in which X is chlorine, Y is hydrogen and Het is 2-methylsulfinyl-4-chloropyridin-3-yl and the combination of R¹ and R² for each compound corresponds to one row of table A.

### Table 230

Compounds of the formula I, in which X is chlorine, Y is hydrogen and Het is 2-methylsulfinyl-4-methylpyridin-3-yl and the combination of R¹ and R² for each compound corresponds to one row of table A.

### Table 231

Compounds of the formula I, in which X is chlorine, Y is hydrogen and Het is 2-methylsulfonyl-4-fluoropyridin-3-yl and the combination of R¹ and R² for each compound corresponds to one row of table A.

### Table 232

Compounds of the formula I, in which X is chlorine, Y is hydrogen and Het is 2-methylsulfonyl-4-chloropyridin-3-yl and the combination of R¹ and R² for each compound corresponds to one row of table A.

### Table 233

Compounds of the formula I, in which X is chlorine, Y is hydrogen and Het is 2-methylsulfonyl-4-methylpyridin-3-yl and the combination of R¹ and R² for each compound corresponds to one row of table A.

### Table 234

Compounds of the formula I, in which X is chlorine, Y is hydrogen and Het is 2-nitro-4-fluoropyridin-3-yl and the combination of R¹ and R² for each compound corresponds to one row of table A.

### Table 235

Compounds of the formula I, in which X is chlorine, Y is hydrogen and Het is 2-nitro-4-chloropyridin-3-yl and the combination of R¹ and R² for each compound corresponds to one row of table A.

### Table 236

Compounds of the formula I, in which X is chlorine, Y is hydrogen and Het is 2-nitro-4-methylpyridin-3-yl and the combination of R¹ and R² for each compound corresponds to one row of table A.

### Table 237

Compounds of the formula I, in which X is chlorine, Y is hydrogen and Het is 2-methoxymethyl-4-fluoropyridin-3-yl and the combination of R¹ and R² for each compound corresponds to one row of table A.

### Table 238

Compounds of the formula I, in which X is chlorine, Y is hydrogen and Het is 2-methoxymethyl-4-chloropyridin-3-yl and the combination of R¹ and R² for each compound corresponds to one row of table A.

### Table 239

Compounds of the formula I, in which X is chlorine, Y is hydrogen and Het is 2-methoxymethyl-4-methylpyridin-3-yl and the combination of R¹ and R² for each compound corresponds to one row of table A.

### Table 240

Compounds of the formula I, in which X is chlorine, Y is hydrogen and Het is 4,6-difluoropyridin-3-yl and the combination of R¹ and R² for each compound corresponds to one row of table A.

### Table 241

Compounds of the formula I, in which X is chlorine, Y is hydrogen and Het is 4-fluoro-6-chloropyridin-3-yl and the combination of R¹ and R² for each compound corresponds to one row of table A.

### Table 242

Compounds of the formula I, in which X is chlorine, Y is hydrogen and Het is 4-fluoro-6-bromopyridin-3-yl and the combination of R¹ and R² for each compound corresponds to one row of table A.

### Table 243

Compounds of the formula I, in which X is chlorine, Y is hydrogen and Het is 4-fluoro-6-methylpyridin-3-yl and the combination of R¹ and R² for each compound corresponds to one row of table A.

### Table 244

Compounds of the formula I, in which X is chlorine, Y is hydrogen and Het is 4-fluoro-6-ethylpyridin-3-yl and the combination of R¹ and R² for each compound corresponds to one row of table A.

### Table 245

Compounds of the formula I, in which X is chlorine, Y is hydrogen and Het is 4-fluoro-6-methoxypyridin-3-yl and the combination of R¹ and R² for each compound corresponds to one row of table A.

### Table 246

Compounds of the formula I, in which X is chlorine, Y is hydrogen and Het is 4-fluoro-6-cyanopyridin-3-yl and the combination of R¹ and R² for each compound corresponds to one row of table A.

### Table 247

Compounds of the formula I, in which X is chlorine, Y is hydrogen and Het is 4-fluoro-6-methoxycarbonylpyridin-3-yl and the combination of R¹ and R² for each compound corresponds to one row of table A.

### Table 248

Compounds of the formula I, in which X is chlorine, Y is hydrogen and Het is 4-fluoro-6-ethoxycarbonylpyridin-3-yl and the combination of R¹ and R² for each compound corresponds to one row of table A.

### Table 249

Compounds of the formula I, in which X is chlorine, Y is hydrogen and Het is 4-fluoro-6-carbamoylpyridin-3-yl and the combination of R¹ and R² for each compound corresponds to one row of table A.

### Table 250

Compounds of the formula I, in which X is chlorine, Y is hydrogen and Het is 4-fluoro-6-methylcarbamoylpyridin-3-yl and the combination of R¹ and R² for each compound corresponds to one row of table A.

### Table 251

Compounds of the formula I, in which X is chlorine, Y is hydrogen and Het is 4-fluoro-6-dimethylcarbamoylpyridin-3-yl and the combination of R¹ and R² for each compound corresponds to one row of table A.

### Table 252

Compounds of the formula I, in which X is chlorine, Y is hydrogen and Het is 4-fluoro-6-thiocarbamoylpyridin-3-yl and the combination of R¹ and R² for each compound corresponds to one row of table A.

### Table 253

Compounds of the formula I, in which X is chlorine, Y is hydrogen and Het is 4-fluoro-6-nitropyridin-3-yl and the combination of R¹ and R² for each compound corresponds to one row of table A.

### Table 254

Compounds of the formula I, in which X is chlorine, Y is hydrogen and Het is 4-fluoro-6-acetylpyridin-3-yl and the combination of R¹ and R² for each compound corresponds to one row of table A.

### Table 255

Compounds of the formula I, in which X is chlorine, Y is hydrogen and Het is 4,6-dichloropyridin-3-yl and the combination of R¹ and R² for each compound corresponds to one row of table A.

### Table 256

Compounds of the formula I, in which X is chlorine, Y is hydrogen and Het is 4-chloro-6-fluoropyridin-3-yl and the combination of R¹ and R² for each compound corresponds to one row of table A.

### Table 257

Compounds of the formula I, in which X is chlorine, Y is hydrogen and Het is 4-chloro-6-bromopyridin-3-yl and the combination of R¹ and R² for each compound corresponds to one row of table A.

### Table 258

Compounds of the formula I, in which X is chlorine, Y is hydrogen and Het is 4-chloro-6-methylpyridin-3-yl and the combination of R¹ and R² for each compound corresponds to one row of table A.

### Table 259

Compounds of the formula I, in which X is chlorine, Y is hydrogen and Het is 4-chloro-6-ethylpyridin-3-yl and the combination of R¹ and R² for each compound corresponds to one row of table A.

### Table 260

Compounds of the formula I, in which X is chlorine, Y is hydrogen and Het is 4-chloro-6-methoxypyridin-3-yl and the combination of R¹ and R² for each compound corresponds to one row of table A.

### Table 261

Compounds of the formula I, in which X is chlorine, Y is hydrogen and Het is 4-chloro-6-cyanopyridin-3-yl and the combination of R¹ and R² for each compound corresponds to one row of table A.

### Table 262

Compounds of the formula I, in which X is chlorine, Y is hydrogen and Het is 4-chloro-6-methoxycarbonylpyridin-3-yl and the combination of R¹ and R² for each compound corresponds to one row of table A.

### Table 263

Compounds of the formula I, in which X is chlorine, Y is hydrogen and Het is 4-chloro-6-ethoxycarbonylpyridin-3-yl and the combination of R¹ and R² for each compound corresponds to one row of table A.

### Table 264

Compounds of the formula I, in which X is chlorine, Y is hydrogen and Het is 4-chloro-6-carbamoylpyridin-3-yl and the combination of R¹ and R² for each compound corresponds to one row of table A.

### Table 265

Compounds of the formula I, in which X is chlorine, Y is hydrogen and Het is 4-chloro-6-methylcarbamoylpyridin-3-yl and the combination of R¹ and R² for each compound corresponds to one row of table A.

### Table 266

Compounds of the formula I, in which X is chlorine, Y is hydrogen and Het is 4-chloro-6-dimethylcarbamoylpyridin-3-yl and the combination of R¹ and R² for each compound corresponds to one row of table A.

### Table 267

Compounds of the formula I, in which X is chlorine, Y is hydrogen and Het is 4-chloro-6-thiocarbamoylpyridin-3-yl and the combination of R¹ and R² for each compound corresponds to one row of table A.

### Table 268

Compounds of the formula I, in which X is chlorine, Y is hydrogen and Het is 4-chloro-6-nitropyridin-3-yl and the combination of R¹ and R² for each compound corresponds to one row of table A.

### Table 269

Compounds of the formula I, in which X is chlorine, Y is hydrogen and Het is 4-chloro-6-acetylpyridin-3-yl and the combination of R¹ and R² for each compound corresponds to one row of table A.

### Table 270

Compounds of the formula I, in which X is chlorine, Y is hydrogen and Het is 4-methyl-6-fluoropyridin-3-yl and the combination of R¹ and R² for each compound corresponds to one row of table A.

### Table 271

Compounds of the formula I, in which X is chlorine, Y is hydrogen and Het is 4-methyl-6-chloropyridin-3-yl and the combination of R¹ and R² for each compound corresponds to one row of table A.

### Table 272

Compounds of the formula I, in which X is chlorine, Y is hydrogen and Het is 4-methyl-6-bromopyridin-3-yl and the combination of R¹ and R² for each compound corresponds to one row of table A.

### Table 273

Compounds of the formula I, in which X is chlorine, Y is hydrogen and Het is 4,6-dimethylpyridin-3-yl and the combination of R¹ and R² for each compound corresponds to one row of table A.

### Table 274

Compounds of the formula I, in which X is chlorine, Y is hydrogen and Het is 4-methyl-6-ethylpyridin-3-yl and the combination of R¹ and R² for each compound corresponds to one row of table A.

### Table 275

Compounds of the formula I, in which X is chlorine, Y is hydrogen and Het is 4-methyl-6-methoxypyridin-3-yl and the combination of R¹ and R² for each compound corresponds to one row of table A.

### Table 276

Compounds of the formula I, in which X is chlorine, Y is hydrogen and Het is 4-methyl-6-cyanopyridin-3-yl and the combination of R¹ and R² for each compound corresponds to one row of table A.

### Table 277

Compounds of the formula I, in which X is chlorine, Y is hydrogen and Het is 4-methyl-6-methoxycarbonylpyridin-3-yl and the combination of R¹ and R² for each compound corresponds to one row of table A.

### Table 278

Compounds of the formula I, in which X is chlorine, Y is hydrogen and Het is 4-methyl-6-ethoxycarbonylpyridin-3-yl and the combination of R¹ and R² for each compound corresponds to one row of table A.

### Table 279

Compounds of the formula I, in which X is chlorine, Y is hydrogen and Het is 4-methyl-6-carbamoylpyridin-3-yl and the combination of R¹ and R² for each compound corresponds to one row of table A.

### Table 280

Compounds of the formula I, in which X is chlorine, Y is hydrogen and Het is 4-methyl-6-methylcarbamoylpyridin-3-yl and the combination of R¹ and R² for each compound corresponds to one row of table A.

### Table 281

Compounds of the formula I, in which X is chlorine, Y is hydrogen and Het is 4-methyl-6-dimethylcarbamoylpyridin-3-yl and the combination of R¹ and R² for each compound corresponds to one row of table A.

### Table 282

Compounds of the formula I, in which X is chlorine, Y is hydrogen and Het is 4-methyl-6-thiocarbamoylpyridin-3-yl and the combination of R¹ and R² for each compound corresponds to one row of table A.

### Table 283

Compounds of the formula I, in which X is chlorine, Y is hydrogen and Het is 4-methyl-6-nitropyridin-3-yl and the combination of R¹ and R² for each compound corresponds to one row of table A.

### Table 284

Compounds of the formula I, in which X is chlorine, Y is hydrogen and Het is 4-methyl-6-acetylpyridin-3-yl and the combination of R¹ and R² for each compound corresponds to one row of table A.

### Table 285

Compounds of the formula I, in which X is chlorine, Y is hydrogen and Het is 4-bromo-6-fluoropyridin-3-yl and the combination of R¹ and R² for each compound corresponds to one row of table A.

### Table 286

Compounds of the formula I, in which X is chlorine, Y is hydrogen and Het is 4-bromo-6-chloropyridin-3-yl and the combination of R¹ and R² for each compound corresponds to one row of table A.

### Table 287

Compounds of the formula I, in which X is chlorine, Y is hydrogen and Het is 4-bromo-6-methylpyridin-3-yl and the combination of R¹ and R² for each compound corresponds to one row of table A.

### Table 288

Compounds of the formula I, in which X is chlorine, Y is hydrogen and Het is 4-methoxy-6-fluoropyridin-3-yl and the combination of R¹ and R² for each compound corresponds to one row of table A.

### Table 289

Compounds of the formula I, in which X is chlorine, Y is hydrogen and Het is 4-methoxy-6-chloropyridin-3-yl and the combination of R¹ and R² for each compound corresponds to one row of table A.

### Table 290

Compounds of the formula I, in which X is chlorine, Y is hydrogen and Het is 4-methoxy-6-methylpyridin-3-yl and the combination of R¹ and R² for each compound corresponds to one row of table A.

### Table 291

Compounds of the formula I, in which X is chlorine, Y is hydrogen and Het is 4-methylsulfanyl-6-fluoropyridin-3-yl and the combination of R¹ and R² for each compound corresponds to one row of table A.

### Table 292

Compounds of the formula I, in which X is chlorine, Y is hydrogen and Het is 4-methylsulfanyl-6-chloropyridin-3-yl and the combination of R¹ and R² for each compound corresponds to one row of table A.

### Table 293

Compounds of the formula I, in which X is chlorine, Y is hydrogen and Het is 4-methylsulfanyl-6-methylpyridin-3-yl and the combination of R¹ and R² for each compound corresponds to one row of table A.

### Table 294

Compounds of the formula I, in which X is chlorine, Y is hydrogen and Het is 4-methylsulfinyl-6-fluoropyridin-3-yl and the combination of R¹ and R² for each compound corresponds to one row of table A.

### Table 295

Compounds of the formula I, in which X is chlorine, Y is hydrogen and Het is 4-methylsulfinyl-6-chloropyridin-3-yl and the combination of R¹ and R² for each compound corresponds to one row of table A.

### Table 296

Compounds of the formula I, in which X is chlorine, Y is hydrogen and Het is 4-methylsulfinyl-6-methylpyridin-3-yl and the combination of R¹ and R² for each compound corresponds to one row of table A.

### Table 297

Compounds of the formula I, in which X is chlorine, Y is hydrogen and Het is 4-methylsulfonyl-6-fluoropyridin-3-yl and the combination of R¹ and R² for each compound corresponds to one row of table A.

### Table 298

Compounds of the formula I, in which X is chlorine, Y is hydrogen and Het is 4-methylsulfonyl-6-chloropyridin-3-yl and the combination of R¹ and R² for each compound corresponds to one row of table A.

### Table 299

Compounds of the formula I, in which X is chlorine, Y is hydrogen and Het is 4-methylsulfonyl-6-methylpyridin-3-yl and the combination of R¹ and R² for each compound corresponds to one row of table A.

### Table 300

Compounds of the formula I, in which X is chlorine, Y is hydrogen and Het is 4-nitro-6-fluoropyridin-3-yl and the combination of R¹ and R² for each compound corresponds to one row of table A.

### Table 301

Compounds of the formula I, in which X is chlorine, Y is hydrogen and Het is 4-nitro-6-chloropyridin-3-yl and the combination of R¹ and R² for each compound corresponds to one row of table A.

### Table 302

Compounds of the formula I, in which X is chlorine, Y is hydrogen and Het is 4-nitro-6-methylpyridin-3-yl and the combination of R¹ and R² for each compound corresponds to one row of table A.

### Table 303

Compounds of the formula I, in which X is chlorine, Y is hydrogen and Het is 4-methoxymethyl-6-fluoropyridin-3-yl and the combination of R¹ and R² for each compound corresponds to one row of table A.

### Table 304

Compounds of the formula I, in which X is chlorine, Y is hydrogen and Het is 4-methoxymethyl-6-chloropyridin-3-yl and the combination of R¹ and R² for each compound corresponds to one row of table A.

### Table 305

Compounds of the formula I, in which X is chlorine, Y is hydrogen and Het is 4-methoxymethyl-6-methylpyridin-3-yl and the combination of R¹ and R² for each compound corresponds to one row of table A.

### Table 306

Compounds of the formula I, in which X is chlorine, Y is hydrogen and Het is 2,4-dichloro-6-methylpyridin-3-yl and the combination of R¹ and R² for each compound corresponds to one row of table A.

### Table 307

Compounds of the formula I, in which X is methyl, Y is hydrogen and Het is 2-pyridinyl and the combination of R¹ and R² for each compound corresponds to one row of table A.

### Table 308

Compounds of the formula I, in which X is methyl, Y is hydrogen and Het is 5-nitropyridin-2-yl and the combination of R¹ and R² for each compound corresponds to one row of table A.

### Table 309

Compounds of the formula I, in which X is methyl, Y is hydrogen and Het is 5-cyanopyridin-2-yl and the combination of R¹ and R² for each compound corresponds to one row of table A.

### Table 310

Compounds of the formula I, in which X is methyl, Y is hydrogen and Het is 5-methoxycarbonylpyridin-2-yl and the combination of R¹ and R² for each compound corresponds to one row of table A.

### Table 311

Compounds of the formula I, in which X is methyl, Y is hydrogen and Het is 5-methylpyridin-2-yl and the combination of R¹ and R² for each compound corresponds to one row of table A.

### Table 312

Compounds of the formula I, in which X is methyl, Y is hydrogen and Het is 4-methylpyridin-2-yl and the combination of R¹ and R² for each compound corresponds to one row of table A.

### Table 313

Compounds of the formula I, in which X is methyl, Y is hydrogen and Het is 3-methylpyridin-2-yl and the combination of R¹ and R² for each compound corresponds to one row of table A.

### Table 314

Compounds of the formula I, in which X is methyl, Y is hydrogen and Het is 3-ethylpyridin-2-yl and the combination of R¹ and R² for each compound corresponds to one row of table A.

### Table 315

Compounds of the formula I, in which X is methyl, Y is hydrogen and Het is 6-methylpyridin-2-yl and the combination of R¹ and R² for each compound corresponds to one row of table A.

### Table 316

Compounds of the formula I, in which X is methyl, Y is hydrogen and Het is 5-trifluoromethylpyridin-2-yl and the combination of R¹ and R² for each compound corresponds to one row of table A.

### Table 317

Compounds of the formula I, in which X is methyl, Y is hydrogen and Het is 3-trifluoromethylpyridin-2-yl and the combination of R¹ and R² for each compound corresponds to one row of table A.

### Table 318

Compounds of the formula I, in which X is methyl, Y is hydrogen and Het is 5-fluoropyridin-2-yl and the combination of R¹ and R² for each compound corresponds to one row of table A.

### Table 319

Compounds of the formula I, in which X is methyl, Y is hydrogen and Het is 3-fluoropyridin-2-yl and the combination of R¹ and R² for each compound corresponds to one row of table A.

### Table 320

Compounds of the formula I, in which X is methyl, Y is hydrogen and Het is 3,5-difluoropyridin-2-yl and the combination of R¹ and R² for each compound corresponds to one row of table A.

### Table 321

Compounds of the formula I, in which X is methyl, Y is hydrogen and Het is 3,5-dichloropyridin-2-yl and the combination of R¹ and R² for each compound corresponds to one row of table A.

### Table 322

Compounds of the formula I, in which X is methyl, Y is hydrogen and Het is pyridin-3-yl and the combination of R¹ and R² for each compound corresponds to one row of table A.

### Table 323

Compounds of the formula I, in which X is methyl, Y is hydrogen and Het is pyridin-4-yl and the combination of R¹ and R² for each compound corresponds to one row of table A.

### Table 324

Compounds of the formula I, in which X is methyl, Y is hydrogen and Het is pyrazin-2-yl and the combination of R¹ and R² for each compound corresponds to one row of table A.

### Table 325

Compounds of the formula I, in which X is methyl, Y is hydrogen and Het is pyridazin-3-yl and the combination of R¹ and R² for each compound corresponds to one row of table A.

### Table 326

Compounds of the formula I, in which X is methyl, Y is hydrogen and Het is 6-chloropyridazin-3-yl and the combination of R¹ and R² for each compound corresponds to one row of table A.

### Table 327

Compounds of the formula I, in which X is methyl, Y is hydrogen and Het is 6-methoxypyridazin-3-yl and the combination of R¹ and R² for each compound corresponds to one row of table A.

### Table 328

Compounds of the formula I, in which X is methyl, Y is hydrogen and Het is 1,3,5-triazin-2-yl and the combination of R¹ and R² for each compound corresponds to one row of table A.

### Tables 329 to 612

Compounds of the formula I, in which X is methyl, Y is hydrogen and Het has the meanings given in tables 23 to 306 and the combination of R¹ and R² for each compound corresponds to one row of table A.

### Tables 613 to 918

Compounds of the formula I, in which X is methoxy, Y is hydrogen and Het has the meanings given in tables 1 to 306 and the combination of R¹ and R² for each compound corresponds to one row of table A.

### Tables 919 to 1124

Compounds of the formula I, in which X is cyano, Y is hydrogen and Het has the meanings given in tables 1 to 306 and the combination of R¹ and R² for each compound corresponds to one row of table A.

### Tables 1125 to 1430

Compounds of the formula I, in which X is methylthio, Y is hydrogen and Het has the meanings given in tables 1 to 306 and the combination of R¹ and R² for each compound corresponds to one row of table A.

TABLE A

| No. | R¹ | R² |
|---|---|---|
| A-1 | H | H |
| A-2 | CH₃ | H |
| A-3 | CH₃ | CH₃ |
| A-4 | CH₂CH₃ | H |
| A-5 | CH₂CH₃ | CH₃ |
| A-6 | CH₂CH₃ | CH₂CH₃ |
| A-7 | CH₂CF₃ | H |
| A-8 | CH₂CF₃ | CH₃ |
| A-9 | CH₂CF₃ | CH₂CH₃ |
| A-10 | CH₂CCl₃ | H |
| A-11 | CH₂CCl₃ | CH₃ |
| A-12 | CH₂CCl₃ | CH₂CH₃ |
| A-13 | CH₂CH₂CH₃ | H |
| A-14 | CH₂CH₂CH₃ | CH₃ |
| A-15 | CH₂CH₂CH₃ | CH₂CH₃ |
| A-16 | CH₂CH₂CH₃ | CH₂CH₂CH₃ |
| A-17 | CH(CH₃)₂ | H |
| A-18 | CH(CH₃)₂ | CH₃ |
| A-19 | CH(CH₃)₂ | CH₂CH₃ |
| A-20 | CH₂CH(CH₃)₂ | H |
| A-21 | CH₂CH(CH₃)₂ | CH₃ |
| A-22 | CH₂CH(CH₃)₂ | CH₂CH₃ |
| A-23 | CH₂CH(CH₃)₂ | CH₂CH₂CH₃ |
| A-24 | CH₂CH₂CH₂CH₃ | H |
| A-25 | CH₂CH₂CH₂CH₃ | CH₃ |
| A-26 | CH₂CH₂CH₂CH₃ | CH₂CH₃ |
| A-27 | CH₂CH₂CH₂CH₃ | CH₂CH₂CH₃ |
| A-28 | CH₂CH₂CH₂CH₃ | CH₂CH₂CH₂CH₃ |
| A-29 | (±)CH(CH₃)—CH₂CH₃ | H |
| A-30 | (±)CH(CH₃)—CH₂CH₃ | CH₃ |
| A-31 | (±)CH(CH₃)—CH₂CH₃ | CH₂CH₃ |
| A-32 | (S)CH(CH₃)—CH₂CH₃ | H |
| A-33 | (S)CH(CH₃)—CH₂CH₃ | CH₃ |
| A-34 | (S)CH(CH₃)—CH₂CH₃ | CH₂CH₃ |
| A-35 | (R)CH(CH₃)—CH₂CH₃ | H |
| A-36 | (R)CH(CH₃)—CH₂CH₃ | CH₃ |
| A-37 | (R)CH(CH₃)—CH₂CH₃ | CH₂CH₃ |
| A-38 | (±)CH(CH₃)—CH(CH₃)₂ | H |
| A-39 | (±)CH(CH₃)—CH(CH₃)₂ | CH₃ |
| A-40 | (±)CH(CH₃)—CH(CH₃)₂ | CH₂CH₃ |
| A-41 | (S)CH(CH₃)—CH(CH₃)₂ | H |
| A-42 | (S)CH(CH₃)—CH(CH₃)₂ | CH₃ |
| A-43 | (S)CH(CH₃)—CH(CH₃)₂ | CH₂CH₃ |
| A-44 | (R)CH(CH₃)—CH(CH₃)₂ | H |
| A-45 | (R)CH(CH₃)—CH(CH₃)₂ | CH₃ |
| A-46 | (R)CH(CH₃)—CH(CH₃)₂ | CH₂CH₃ |
| A-47 | (±)CH(CH₃)—C(CH₃)₃ | H |
| A-48 | (±)CH(CH₃)—C(CH₃)₃ | CH₃ |
| A-49 | (±)CH(CH₃)—C(CH₃)₃ | CH₂CH₃ |
| A-50 | (S)CH(CH₃)—C(CH₃)₃ | H |
| A-51 | (S)CH(CH₃)—C(CH₃)₃ | CH₃ |
| A-52 | (S)CH(CH₃)—C(CH₃)₃ | CH₂CH₃ |
| A-53 | (R)CH(CH₃)—C(CH₃)₃ | H |
| A-54 | (R)CH(CH₃)—C(CH₃)₃ | CH₃ |
| A-55 | (R)CH(CH₃)—C(CH₃)₃ | CH₂CH₃ |
| A-56 | (±)CH(CH₃)—CF₃ | H |
| A-57 | (±)CH(CH₃)—CF₃ | CH₃ |
| A-58 | (±)CH(CH₃)—CF₃ | CH₂CH₃ |
| A-59 | (S)CH(CH₃)—CF₃ | H |
| A-60 | (S)CH(CH₃)—CF₃ | CH₃ |
| A-61 | (S)CH(CH₃)—CF₃ | CH₂CH₃ |
| A-62 | (R)CH(CH₃)—CF₃ | H |
| A-63 | (R)CH(CH₃)—CF₃ | CH₃ |
| A-64 | (R)CH(CH₃)—CF₃ | CH₂CH₃ |
| A-65 | (±)CH(CH₃)—CCl₃ | H |
| A-66 | (±)CH(CH₃)—CCl₃ | CH₃ |
| A-67 | (±)CH(CH₃)—CCl₃ | CH₂CH₃ |
| A-68 | (S)CH(CH₃)—CCl₃ | H |
| A-69 | (S)CH(CH₃)—CCl₃ | CH₃ |
| A-70 | (S)CH(CH₃)—CCl₃ | CH₂CH₃ |
| A-71 | (R)CH(CH₃)—CCl₃ | H |
| A-72 | (R)CH(CH₃)—CCl₃ | CH₃ |
| A-73 | (R)CH(CH₃)—CCl₃ | CH₂CH₃ |
| A-74 | CH₂CF₂CF₃ | H |
| A-75 | CH₂CF₂CF₃ | CH₃ |
| A-76 | CH₂CF₂CF₃ | CH₂CH₃ |
| A-77 | CH₂(CF₂)₂CF₃ | H |
| A-78 | CH₂(CF₂)₂CF₃ | CH₃ |
| A-79 | CH₂(CF₂)₂CF₃ | CH₂CH₃ |
| A-80 | CH₂C(CH₃)═CH₂ | H |
| A-81 | CH₂C(CH₃)═CH₂ | CH₃ |
| A-82 | CH₂C(CH₃)═CH₂ | CH₂CH₃ |
| A-83 | CH₂CH═CH₂ | H |
| A-84 | CH₂CH═CH₂ | CH₃ |
| A-85 | CH₂CH═CH₂ | CH₂CH₃ |
| A-86 | (±)CH(CH₃)CH═CH₂ | H |
| A-87 | (±)CH(CH₃)CH═CH₂ | CH₃ |
| A-88 | (±)CH(CH₃)CH═CH₂ | CH₂CH₃ |
| A-89 | (R)CH(CH₃)CH═CH₂ | H |
| A-90 | (R)CH(CH₃)CH═CH₂ | CH₃ |
| A-91 | (R)CH(CH₃)CH═CH₂ | CH₂CH₃ |
| A-92 | (S)CH(CH₃)CH═CH₂ | H |
| A-93 | (S)CH(CH₃)CH═CH₂ | CH₃ |
| A-94 | (S)CH(CH₃)CH═CH₂ | CH₂CH₃ |
| A-95 | (±)CH(CH₃)C(CH₃)═CH₂ | H |
| A-96 | (±)CH(CH₃)C(CH₃)═CH₂ | CH₃ |
| A-97 | (±)CH(CH₃)C(CH₃)═CH₂ | CH₂CH₃ |
| A-98 | (S)CH(CH₃)C(CH₃)═CH₂ | H |
| A-99 | (S)CH(CH₃)C(CH₃)═CH₂ | CH₃ |
| A-100 | (S)CH(CH₃)C(CH₃)═CH₂ | CH₂CH₃ |
| A-101 | (R)CH(CH₃)C(CH₃)═CH₂ | H |
| A-102 | (R)CH(CH₃)C(CH₃)═CH₂ | CH₃ |
| A-103 | (R)CH(CH₃)C(CH₃)═CH₂ | CH₂CH₃ |
| A-104 | CH₂—C≡CH | H |
| A-105 | CH₂—C≡CH | CH₃ |
| A-106 | CH₂—C≡CH | CH₂CH₃ |
| A-107 | cyclopentyl | H |
| A-108 | cyclopentyl | CH₃ |
| A-109 | cyclopentyl | CH₂CH₃ |
| A-110 | cyclohexyl | H |
| A-111 | cyclohexyl | CH₃ |
| A-112 | cyclohexyl | CH₂CH₃ |
| A-113 | CH₂—C₆H₅ | H |
| A-114 | CH₂—C₆H₅ | CH₃ |
| A-115 | CH₂—C₆H₅ | CH₂CH₃ |
| A-116 | NH₂ | CH₂-c-C₆H₁₁ |
| A-117 | NH₂ | CH₂CH₃ |
| A-118 | NH₂ | CH₂CH₂CH₃ |
| A-119 | NH—CH₂—CH═CH₂ | H |
| A-120 | NH—CH₂—CH═CH₂ | CH₃ |
| A-121 | NH—CH₂—CH═CH₂ | CH₂CH₃ |
| A-122 | NH—C(CH₃)₃ | H |
| A-123 | N(CH₃)₂ | H |
| A-124 | N(CH₃) | H |

| | |
|---|---|
| A-125 | —(CH₂)₂CH═CHCH₂— |
| A-126 | —(CH₂)₂C(CH₃)═CHCH₂— |
| A-127 | —CH(CH₃)CH₂—CH═CHCH₂— |
| A-128 | —(CH₂)₂CH(CH₃)(CH₂)₂— |
| A-129 | —(CH₂)₃CHFCH₂— |
| A-130 | —(CH₂)₂CHF(CH₂)₂— |
| A-131 | —CH₂CHF(CH₂)₃— |
| A-132 | —(CH₂)₂CH(CF₃)(CH₂)₂— |
| A-133 | —(CH₂)₂O(CH₂)₂— |
| A-134 | —(CH₂)₂S(CH₂)₂— |
| A-135 | —(CH₂)₅— |
| A-136 | —(CH₂)₄— |
| A-137 | —CH₂CH═CHCH₂— |
| A-138 | —CH(CH₃)(CH₂)₃— |
| A-139 | —CH₂CH(CH₃)(CH₂)₂— |
| A-140 | —CH(CH₃)—(CH₂)₂—CH(CH₃)— |
| A-141 | —CH(CH₃)—(CH₂)₄— |
| A-142 | —CH₂—CH(CH₃)—(CH₂)₃— |
| A-143 | —(CH₂)—CH(CH₃)—CH₂—CH(CH₃)—CH₂— |
| A-144 | —CH(CH₂CH₃)—(CH₂)₄— |
| A-145 | —(CH₂)₂—CHOH—(CH₂)₂— |
| A-146 | —(CH₂)₆— |
| A-147 | —CH(CH₃)—(CH₂)₅— |
| A-148 | —(CH₂)₂—N(CH₃)—(CH₂)₂— |
| A-149 | —N═CH—CH═CH— |
| A-150 | —N═C(CH₃)—CH═C(CH₃)— |
| A-151 | —N═C(CF₃)—CH═C(CF₃)— |

The compounds of the formula I according to the invention can be obtained by different routes analogously to processes of the prior art known per se, according to the syntheses shown in the schemes below:

Compounds of the formula I in which X is Hal can be prepared, for example, according to the synthesis shown in scheme 1.

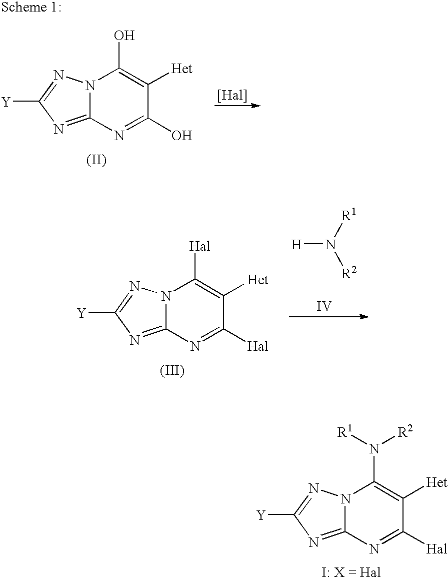

In scheme 1, Y, R¹, R² and Het are as defined above. Hal is halogen, preferably chlorine or bromine.

In a first step, dihydroxytriazolopyrimidines of the formula II are, analogously to methods described in the prior art cited at the outset or in WO-A 94/20501, converted into the dihalo compounds of the formula III by reaction with a halogenating agent [HAL]. Advantageous for use as halogenating agents are phosphorus oxyhalides or phosphorus(V) halides such as phosphorus pentachloride, phosphorus oxybromide or phosphorus oxychloride, or a mixture of phosphorus oxychloride with phosphorus pentachloride. This reaction of II with the halogenating agent is usually carried out at from 0° C. to 150° C., preferably at from 80° C. to 125° C. [cf. also EP-A 770 615]. The reaction can be carried out in the absence of a solvent or in an inert solvent, for example in a halogenated hydrocarbon, such as dichloromethane or dichloroethane, or an aromatic hydrocarbon, such as, for example toluene, xylenes and the like, or in a mixture of the solvents mentioned above.

The reaction of III with amines IV is carried out analogously to the methods described in the prior art cited at the outset or in WO 98/46608, advantageously at temperatures in the range from 0° C. to 70° C., preferably from 10° C. to 35° C. The reaction is preferably carried out in an inert solvent, for example in an ether, such as dioxane, diethyl ether, diisopropyl ether, tert-butyl methyl ether or, in particular, tetrahydrofuran, a halogenated hydrocarbon, such as dichloromethane or dichloroethane, or an aromatic hydrocarbon, such as, for example, toluene, xylenes and the like, or in a mixture of the solvents mentioned above. It is preferred to use a base, such as a tertiary amine, for example triethylamine, biscyclohexylmethylamine, pyridine, picoline or an inorganic base, such as potassium carbonate; it is also possible for excess amine of the formula IV to serve as base.

The amines IV are commercially available or can be prepared by known processes.

The compounds of the formulae II and III and their agriculturally acceptable salts are novel and also form part of the subject-matter of the present invention. The compounds of the formulae II and III and their agriculturally acceptable salts are furthermore distinguished by their activity against plant-damaging fungi. Accordingly, their use for controlling plant-pathogenic fungi and the corresponding method and crop protection compositions comprising at least one compound of the formula II or III also form part of the subject-matter of the present invention.

Dihydroxytriazolopyrimidines of the formula II can be prepared analogously to methods described in the prior art cited at the outset or in Adv. Het. Chem. 57, (1993), 81 ff. by reacting a 3-amino-2H-1,2,4-triazole V with appropriately substituted hetarylmalonates of the formula VI. In formula VI, R is alkyl, preferably C₁-C₆-alkyl, in particular methyl or ethyl. Het and Y are as defined above.

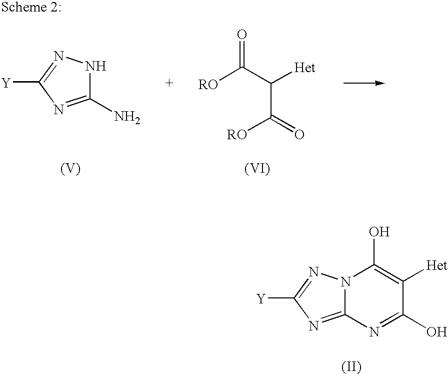

This reaction is usually carried out at temperatures of from 80° C. to 250° C., preferably from 120° C. to 180° C., without solvent or in an inert organic solvent in the presence of a base [cf. EP-A 770 615] or in the presence of acetic acid, under the conditions known from Adv. Het. Chem. Vol. 57, p. 81ff. (1993).

Suitable solvents are aliphatic hydrocarbons, aromatic hydrocarbons, such as toluene, o-, m- and p-xylene, halogenated hydrocarbons, ethers, nitriles, ketones, alcohols, and also N-methylpyrrolidone, dimethyl sulfoxide, dimethylformamide and dimethyl-acetamide. With particular preference, the reaction is carried out without solvent or in chlorobenzene, xylene, dimethyl sulfoxide or N-methylpyrrolidone. It is also possible to use mixtures of the solvents mentioned. If appropriate, it may also be possible to add catalytic amounts of acids, such as p-toluene sulfonic acid, acetic acid or propionic acid.

Suitable bases are, in general, inorganic compounds, such as alkali metal and alkaline earth metal hydroxides, alkali metal and alkaline earth metal oxides, alkali metal and alkaline earth metal hydrides, alkali metal amides, alkali metal and alkaline earth metal carbonates, and also alkali metal bicarbonates, organometallic compounds, in particular alkali metal alkyls, alkylmagnesium halides, and also alkali metal and alkaline earth metal alkoxides and dimethoxymagnesium, moreover organic bases, for example tertiary amines, such as trimethylamine, triethylamine, diisopropylethylamine, tributylamine and N-methylpiperidine, N-methylmorpholine, pyridine, substituted pyridines, such as collidine, lutidine and 4-dimethylaminopyridine, and also bicyclic amines. Particular preference is given to tertiary amines, such as diisopropylethyl-amine, tributylamine, N-methylmorpholine or N-methylpiperidine.

The bases are generally employed in catalytic amounts; however, they can also be employed in equimolar amounts, in excess or, if appropriate, as solvent.

The starting materials are generally reacted with one another in equimolar amounts. In terms of yield, it may be advantageous to employ an excess of base and malonate VI, based on the triazole V.

Some of the hetarylmalonates of the formula VI are novel and also form part of the subject-matter of the present invention, i.e. if Het has 1, 2 or 3 substituents independently of one another selected from the group consisting of hydroxyl, cyanato (OCN), C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoximinoalkyl, C₂-C₁₀-alkenyloximinoalkyl, C₂-C₁₀-alkynyloximinoalkyl, C₂-C₁₀-alkynylcarbonyl, C₃-C₆-cycloalkylcarbonyl, C(═O)A², C(═S)A², a group —C(═N—OR⁷)(NR⁸R⁹) or a group —C(═N—NR¹⁰R¹¹)(NR¹²R¹³), where A², R⁷, R⁸, R⁹, R¹⁰, R¹¹, R¹² and R¹³ are as defined above, where Het may additionally also have 1, 2 or 3 further substituents selected from the group consisting of halogen and cyano, except for malonates of the formula VI in which Het is 5-trifluoromethylpyridin-2-yl, 6-trifluoromethylpyridin-2-yl, 3-chloro-5-trifluoromethylpyridin-2-yl, 3-chloro-6-trifluoromethylpyridin-2-yl, 3-cyano-5-trifluoromethylpyridin-2-yl, 3-cyano-6-isopropylpyridin-2-yl, 4,6-dimethoxy-1,3,5-triazin-2-yl, 3-trifluoromethyl-4-(1,2,2,2-tetrafluoro-1-(trifluoromethyl)ethyl)pyridin-2-yl or 3,4,5-trifluoro-6-(1,2,2,2-tetrafluoro-1-(trifluoromethyl)ethyl)pyridin-2-yl.

Hetarylmalonates of the formula VI can be prepared from hetaryl compounds of the formula VII by reacting one or two equivalents of a carbonic ester or a chloroformate (compound VIII) in the presence of a strong base (see scheme 3).

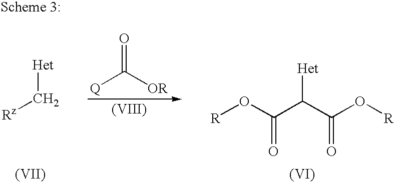

In scheme 3, R^{z} is hydrogen or a C₁-C₄-alkoxycarbonyl group. Q is halogen or C₁-C₄-alkoxy, in particular methoxy or ethoxy. Het has the meanings given above and R is C₁-C₄-alkyl. It will be evident to the person skilled in the art that in the case of R^{z}=H at least two equivalents of the compound VIII have to be employed to achieve complete conversion of VII.

The reaction shown in scheme 3 is usually carried out in the presence of strong bases. If R^{z} is hydrogen, the base employed will usually be an alkali metal amide, such as sodium amide or lithium diisopropylamide, or an organolithium compound, such as phenyllithium or butyllithium. In this case, the base will be employed in an at least equimolar amount, based on the compound VII, to achieve complete conversion. If R^{z} is an alkoxycarbonyl group, the base employed will preferably be an alkali metal alkoxide, for example sodium ethoxide or potassium ethoxide, sodium butoxide or potassium butoxide, sodium methoxide or potassium methoxide. For R^{z}=H, the reaction of VII with VIII can be carried out in one step or in two separate steps, where, in the latter case, the compound VII in which R^{z} is an alkoxycarbonyl group is obtained as intermediate. The reaction of VII with VIII can also be carried out analogously to the method described in J. Med. Chem. 25 (1982), 745.

Advantageously, malonates of the formula VI can also be prepared by reacting appropriate bromohetaryl compounds Br-Het with dialkyl malonates under Cu(I) catalysis [cf. Chemistry Letters (1981), 367-370; EP-A 10 02 788].

Compounds of the formula I in which X is H, C₁-C₄-alkyl, C₂-C₈-alkenyl or C₂-C₈-alkynyl or a corresponding halogenated radical can be prepared, for example, according to the synthesis shown in scheme 4.

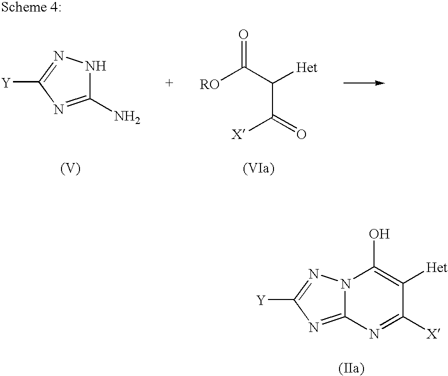

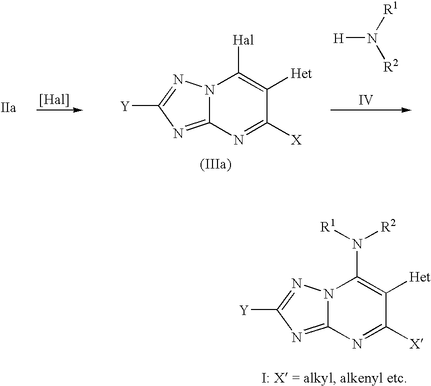

In scheme 4, Y, R¹, R² and Het are as defined above. Hal is halogen, preferably chlorine or bromine. X′ is H, C₁-C₈-alkyl, C₂-C₈-alkenyl or C₂-C₈-alkynyl or a corresponding halogenated radical, and R is C₁-C₄-alkyl. The reactions shown in scheme 4 can be carried out analogously to the reactions illustrated for schemes 1 and 2.

Some of the compounds of the formula VIa are novel and also form part of the subject-matter of the present invention, i.e. if Het has 1, 2 or 3 substituents independently of one another selected from the group consisting of cyano, hydroxyl, cyanato (OCN), C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₂-C₁₀-alkenyloxycarbonyl, C₂-C₁₀-alkynyloxy-carbonyl, C₁-C₈-alkoximinoalkyl, C₂-C₁₀-alkenyloximinoalkyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C₆-cycloalkylcarbonyl, S(═O)ₙA¹, C(═O)A², C(═S)A², a group —C(═N—OR⁷)(NR⁸R⁹) or a group —C(═N—NR¹⁰R¹¹)(NR¹²R¹³), in which A¹, A², R⁷, R⁸, R⁹, R¹⁰, R¹¹, R¹² and R¹³ are as defined above, where Het may additionally also have 1, 2 or 3 further halogen atoms as substituents L, except for compounds VIa in which Het is 5-ethoxycarbonylpyridin-2-yl, 2-methylpyridin-3-yl, 4-chloro-6-methoxy-1,3,5-triazin-2-yl or 4,6-dimethoxy-1,3,5-triazin-2-yl.

It is self-evident that the maximum number of substituents L on the 6-membered aromatic heterocycle in the compounds I, II, IIa, III, IIIa, VI and VIa is 5 minus the ring nitrogens.

Analogously to standard processes in the sense of a mixed ester condensation, the compounds of the formula VIa can be prepared from the corresponding hetarylacetic acid esters by reaction with the appropriate aliphatic alkyl C₂-C₅-carboxylates, such as ethyl acetate, ethyl propionate, ethyl butyrate or ethyl valerate or with a reactive derivative thereof, for example an acid chloride or an acid anhydride, in the presence of a strong base, for example an alkoxide, an alkali metal amide or an organolithium compound, for example analogously to the methods described in J. Chem. Soc. Perkin Trans (1967), 767 or in Eur. J. Org. Chem. (2002), 3986.

Advantageously, compounds of the formula I in which X is cyano, C₁-C₈-alkoxy, C₁-C₈-alkylthio or C₁-C₈-haloalkoxy can also be prepared by reacting compounds I in which X is halogen, preferably chlorine, with compounds M¹-X′ (hereinbelow also referred to as compounds of the formula IX). Depending on the group X′ to be introduced, the compounds of the formula IX are inorganic cyanides, alkoxides, thiolates or haloalkoxides. The reaction is advantageously carried out in an inert solvent. The cation M¹ in formula IX is of little importance; for practical reasons, ammonium salts, tetraalkylammonium salts such as tetramethylammonium or tetraethylammonium salts, or alkali metal salts or alkaline earth metal salts are usually preferred (Scheme 5).

### Scheme 5:

(I)+M¹-X′→(I)

(X=halogen) (IX) {X=X′=CN, C₁-C₄-alkoxy, C₁-C₄-haloalkoxy}

The reaction temperature is usually from 0 to 120° C., preferably from 10 to 40° C. [cf. J. Heterocycl. Chem. 12 (1975), 861-863].

Suitable solvents include ethers, such as dioxane, diethyl ether, methyl tert-butyl ether and, preferably, tetrahydrofuran, halogenated hydrocarbons, such as dichloromethane or dichloroethane, aromatic hydrocarbons, such as toluene and mixtures thereof.

Compounds of the formula I, in which X is C₁-C₈-alkyl, C₁-C₈-haloalkyl, C₂-C₈-alkenyl, C₂-C₈-haloalkenyl, C₂-C₈-alkynyl or C₂-C₈-haloalkynyl can advantageously be prepared by reacting compounds I in which X is halogen with organometallic compounds X^{a}-Mt, where X^{a} is C₁-C₄-alkyl, C₁-C₄-haloalkyl, C₂-C₈-alkenyl, C₂-C₈-haloalkenyl, C₂-C₈-alkynyl or C₂-C₈-haloalkynyl and Mt is lithium, magnesium or zinc. The reaction is preferably carried out in the presence of catalytic or, in particular, at least equimolar amounts of transition metal salts and/or compounds, in particular in the presence of Cu salts, such as Cu(I) halides and, especially, Cu(I) iodide. The reaction is generally carried out in an inert organic solvent, for example one of the ethers mentioned above, in particular tetrahydrofuran, an aliphatic or cycloaliphatic hydrocarbon, such as hexane, cyclohexane and the like, an aromatic hydrocarbon, such as toluene, or in a mixture of these solvents. The temperatures required for this purpose are in the range of from −100 to +100° C. and especially in the range from −80° C. to +40° C. The appropriate methods are known, for example from the prior art cited at the outset (see, for example, WO 03/004465).

In principle, compounds of the formula I, in which X is C₁-C₈-alkyl can also be prepared by reacting compounds I in which X is halogen, in particular chlorine, with malonates of the formula X. This synthesis route is shown in scheme 6. In formula X, X″ is hydrogen or C₁-C₃-alkyl and R is C₁-C₄-alkyl. The compounds I are converted into compounds of the formula XI which are subsequently, after hydrolysis, decarboxylated to give compounds I [cf. U.S. Pat. No. 5,994,360].

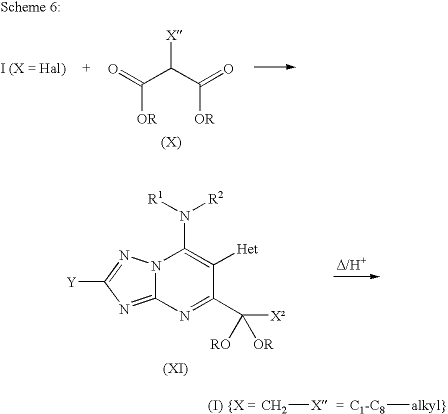

The malonates X are known from the literature, for example from J. Am. Chem. Soc. 64 (1942), 2714; J. Org. Chem. 39 (1974), 2172; Helv. Chim. Acta 61, (1978), 1565, or they can be prepared in accordance with the literature cited.

The subsequent hydrolysis of the ester XI is carried out under generally customary conditions. Depending on the various structural elements, alkaline or acidic hydrolysis of the compounds XI may be advantageous. Under the conditions of ester hydrolysis, there may already be complete or partial decarboxylation to I. Usually, decarboxylation takes place at temperatures of from 20° C. to 180° C., preferably from 50° C. to 120° C., in an inert solvent, if appropriate in the presence of an acid. Suitable acids are hydrochloric acid, sulfuric acid, phosphoric acid, formic acid, acetic acid, p-toluenesulfonic acid. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide; with particular preference, the reaction is carried out in hydrochloric acid or acetic acid. It is also possible to use mixtures of the solvents mentioned.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use during application (for example under the action of light, acids or bases). Such conversions may also take place after use, for example, in the case of treatment of plants, in the treated plants, or in the harmful fungus to be controlled.

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar fungicides, as fungicides for seed dressing and as soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on vegetables, rapeseed, sugar beet, rice and fruit,
Aphanomyces species on sugar beet and vegetables,
Bipolaris and Drechslera species on cereals, corn, rice and lawns,
Blumeria graminis (powdery mildew) on cereals,
Botrytis cinerea (gray mold) on strawberries, vegetables, ornamental plants and grapevines,
Bremia lactucae on lettuce,
Cerospora species on corn, soybeans, rice and sugar beet,
Cochliobolus species on corn, cereals, rice (e.g., Cochliobolus sativus on cereals, Cochliobolus miyabeanus on rice),
Colletotricum species on soybeans and cotton,
Drechslera species on cereals and corn,
Exserohilum species on corn,
Erysiphe cichoracearum and Sphaerotheca fuliginea on cucurbits,
Erysiphe (syn. Uncinula) necator on grapevines,
Fusarium and Verticillium species on various plants,
Gaeumanomyces graminis on cereals,
Gibberella species on cereals and rice (e.g., Gibberella fujikuroi on rice),
Grain staining complex on rice,
Helminthosporium species on corn and rice,
Michrodochium nivale on cereals,
Mycosphaerella species on cereals, bananas and peanuts,
Phakopsara pachyrhizi and Phakopsara meibomiae on soybeans,
Phomopsis species on soybeans and sunflowers,
Phytophthora infestans on potatoes and tomatoes,
Plasmopara viticola on grapevines,
Podosphaera leucotricha on apples,
Pseudocercosporella herpotrichoides on cereals, especially wheats and barley
Pseudoperonospora species on hops and cucumber,
Puccinia species on cereals,
Pyrenophora species on cereals,
Pyricularia oryzae, Corticium sasakii, Sarocladium oryzae, S. attenuatum, Entyloma oryzae on rice,
Pyricularia grisea on lawns and cereals,
Pythium spp. on lawns, rice, corn, cotton, rapeseed, sunflowers, sugar beet, vegetables and other plants,
Rhizoctonia species on cotton, rice, lawns, potatoes, corn, rapeseed, sugar beet, vegetables and other plants,
Sclerotinia species on rapeseed and sunflowers,
Septoria tritici and Stagonospora nodorum on wheat,
Setospaeria species on corn and lawns,
Sphacelotheca reilinia on corn,
Thievaliopsis species on soybeans and cotton,
Tilletia species on cereals,
Ustilago species on cereals, and sugar cane, and
Venturia species (scab) on apples and pears.

The compounds (I) are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

In addition, the compounds of the formula (I) can also be used in crops which, owing to breeding including genetical engineering, are tolerant to attack by insects or fungi.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

Seed can be treated by methods known to the person skilled in the art, such as, for example, seed dressing, seed coating, seed dusting, seed soaking and seed pelleting.

In seed treatment, amounts of active compound of from 1 to 1000 g, preferably from 1 to 200 g, in particular from 5 to 100 g, per 100 kg of seed are generally required.

Accordingly, the present invention furthermore provides seed comprising a compound of the formula (I) according to the invention in an amount of from 1 to 1000 g per 100 kg.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used,
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

Formulations for seed treatment may further comprise binders and/or gelants and if appropriate dyes.

Binders can be added to increase the adhesion of the active compounds to the seed after the treatment. Suitable binders are for example EO/PO block copolymer surfactants, but also polyvinyl alcohols, polyvinylpyrrolidones, polyacrylates, polymethacrylates, polybutenes, polyisobutylenes, polystyrenes, polyethyleneamines, polyethyleneamides, polyethyleneimines (Lupasol®, Polymin®), polyethers, polyurethanes, polyvinyl acetates, Tylose and copolymers of these polymers. A suitable gelant is for example carrageen (Satiagel®).

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

Examples of Formulations are:

1. products for dilution with water
A Water-soluble concentrates (SL)
10 parts by weight of a compound according to the invention are dissolved in water or in a water-soluble solvent. As an alternative, wetters or other auxiliaries are added. The active compound dissolves upon dilution with water.
   B Dispersible concentrates (DC)
20 parts by weight of a compound according to the invention are dissolved in cyclohexanone with addition of a dispersant, for example polyvinylpyrrolidone. Dilution with water gives a dispersion.
   C Emulsifiable concentrates (EC)
15 parts by weight of a compound according to the invention are dissolved in xylene with addition of calcium dodecylbenzenesulfonate and castor oil ethoxylate (in each case 5%). Dilution with water gives an emulsion.

### D Emulsions (EW, EO)

40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion.

### E Suspensions (SC, OD)

In an agitated ball mill, 20 parts by weight of a compound according to the invention are comminuted with addition of dispersants, wetters and water or an organic solvent to give a fine active compound suspension. Dilution with water gives a stable suspension of the active compound.
   F Water-dispersible granules and water-soluble granules (WG, SG)
50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound.
   G Water-dispersible powders and water-soluble powders (WP, SP)
75 parts by weight of a compound according to the invention are ground in a rotor-stator mill with addition of dispersants, wetters and silica gel. Dilution with water gives a stable dispersion or solution of the active compound.
   2. Products to be applied undiluted
   H Dustable powders (DP)
5 parts by weight of a compound according to the invention are ground finely and mixed intimately with 95% of finely divided kaolin. This gives a dustable product.

### I Granules (GR, FG, GG, MG)

0.5 part by weight of a compound according to the invention is ground finely and associated with 95.5% carriers. Current methods are extrusion, spray-drying or the fluidized bed. This gives granules to be applied undiluted.
   J ULV solutions (UL)
10 parts by weight of a compound according to the invention are dissolved in an organic solvent, for example xylene. This gives a product to be applied undiluted.

Seed treatment typically utilizes water-soluble concentrates, suspensions, dusts, water-dispersible and water-soluble powders, emulsions, emulsifiable concentrates and gel formulations. These formulations can be applied neat or preferably diluted to the seed. The application can take place prior to sowing.

Preference is given to using suspension formulations for seed treatment. Such formulations typically comprise from 1 to 800 g/l of active compound, from 1 to 200 g/l of surfactants, from 0 to 200 g/l of antifreeze, from 0 to 400 g/l of binder, from 0 to 200 g/l of dyes and solvent, preferably water.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

For the treatment of seed, the formulations in question may be diluted two- to ten-fold, resulting in active compound concentrations of from 0.01 to 60% by weight, preferably from 0.1 to 40% by weight, in the finished ready-to-use preparations.

Various types of oils, wetters, adjuvants, herbicides, fungicides, other pesticides, or bactericides may be added to the active compounds, if appropriate not until immediately prior to use (tank mix). These agents can be admixed with the agents according to the invention in a weight ratio of 1:100 to 100:1, preferably 1:10 to 10:1.

Suitable adjuvants in this sense are in particular: organically modified polysiloxanes, for example Break Thru S 240®; alcohol alkoxylates, for example Atplus 245®, Atplus MBA 1303®, Plurafac LF 300® and Lutensol ON 30®; EO/PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention can, in the use form as fungicides, also be present together with other active compounds, e.g. with herbicides, pesticides (such as insecticides and acaricides), growth regulators, fungicides or else with fertilizers. Mixing the compounds I or the compositions comprising them in the application form as fungicides with one or more active substances, in particular other fungicides results in many cases in a broadening of the spectrum of activity being obtained or resistance development can be prevented thereby. In many cases synergistic effects are achieved.

The following list of fungicides, in conjunction with which the compounds according to the invention can be used, is intended to illustrate the possible combinations but does not limit them:

### 1. Strobilurins

azoxystrobin, dimoxystrobin, enestroburin, fluoxastrobin, kresoxim-methyl, metominostrobin, picoxystrobin, pyraclostrobin, trifloxystrobin, orysastrobin, methyl (2-chloro-5-[1-(3-methylbenzyloxyimino)ethyl]benzyl)carbamate, methyl (2-chloro-5-[1-(6-methylpyridin-2-ylmethoxyimino)ethyl]benzyl)carbamate, methyl 2-(ortho-((2,5-dimethylphenyloxymethylene)phenyl)-3-methoxyacrylate;

### 2. Carboxamides

carboxanilides: benalaxyl, benodanil, boscalid, carboxin, mepronil, fenfuram, fenhexamid, flutolanil, furametpyr, metalaxyl, ofurace, oxadixyl, oxycarboxin, penthiopyrad, thifluzamide, tiadinil, N-(4′-bromobiphenyl-2-yl)-4-difluoromethyl-2-methylthiazole-5-carboxamide, N-(4′-trifluoromethylbiphenyl-2-yl)-4-difluoromethyl-2-methylthiazole-5-carboxamide, N-(4′-chloro-3′-fluorobiphenyl-2-yl)-4-difluoromethyl-2-methylthiazole-5-carboxamide, N-(3′,4′-dichloro-4-fluorobiphenyl-2-yl)-3-difluoromethyl-1-methylpyrazole-4-carboxamide, N-(2-cyanophenyl)-3,4-dichloroisothiazole-5-carboxamide;
carboxylic acid morpholides: dimethomorph, flumorph;
benzamides: flumetover, fluopicolide (picobenzamid), zoxamide;
other carboxamides: carpropamid, diclocymet, mandipropamid, N-(2-(4-[3-(4-chlorophenyl)prop-2-ynyloxy]-3-methoxyphenyl)ethyl)-2-methanesulfonylamino-3-methylbutyramide, N-(2-(4-[3-(4-chlorophenyl)prop-2-ynyloxy]-3-methoxyphenyl)-ethyl)-2-ethanesulfonylamino-3-methylbutyramide;

### 3. Azoles

triazoles: bitertanol, bromuconazole, cyproconazole, difenoconazole, diniconazole, enilconazole, epoxiconazole, fenbuconazole, flusilazole, fluquinconazole, flutriafol, hexaconazole, imibenconazole, ipconazole, metconazole, myclobutanil, penconazole, propiconazole, prothioconazole, simeconazole, tebuconazole, tetraconazole, triadimenol, triadimefon, triticonazole;
imidazoles: cyazofamid, imazalil, pefurazoate, prochloraz, triflumizole;
benzimidazoles: benomyl, carbendazim, fuberidazole, thiabendazole;
others: ethaboxam, etridiazole, hymexazole;
   4. Nitrogenous heterocyclyl compounds:
pyridines: fluazinam, pyrifenox, 3-[5-(4-chlorophenyl)-2,3-dimethylisoxazolidin-3-yl]pyridine;
pyrimidines: bupirimate, cyprodinil, ferimzone, fenarimol, mepanipyrim, nuarimol, pyrimethanil;
piperazines: triforine;
pyrroles: fludioxonil, fenpiclonil;
morpholines: aldimorph, dodemorph, fenpropimorph, tridemorph;
dicarboximides: iprodione, procymidone, vinclozolin;
others: acibenzolar-5-methyl, anilazine, captan, captafol, dazomet, diclomezine, fenoxanil, folpet, fenpropidin, famoxadone, fenamidone, octhilinone, probenazole, proquinazid, pyroquilon, quinoxyfen, tricyclazole, 5-chloro-7-(4-methylpiperidin-1-yl)-6-(2,4,6-trifluorophenyl)-[1,2,4]triazolo[1,5-a]pyrimidine, 2-butoxy-6-iodo-3-propylchromen-4-one, N N-dimethyl-3-(3-bromo-6-fluoro-2-methylindole-1-sulfonyl)-[1,2,4]triazole-1-sulfonamide;
   5. Carbamates and dithiocarbamates
dithiocarbamates: ferbam, mancozeb, maneb, metiram, metam, propineb, thiram, zineb, ziram;
carbamates: diethofencarb, flubenthiavalicarb, iprovalicarb, propamocarb, methyl 3-(4-chlorophenyl)-3-(2-isopropoxycarbonylamino)pro-pionate, 4-fluorophenyl N-(1-(1-(4-cyanophenyl)ethanesulfonyl)but-2-yl)-carbamate;
   6. Other fungicides
guanidines: dodine, iminoctadine, guazatine;
antibiotics: kasugamycin, polyoxins, streptomycin, validamycin A;
organometallic compounds: fentin salts;
sulfur-containing heterocyclyl compounds: isoprothiolane, dithianon;
organophosphorous compounds: edifenphos, fosetyl, fosetyl-aluminum, iprobenfos, pyrazophos, tolclofos-methyl, phosphorous acid and its salts;
organochlorine compounds: thiophanate methyl, chlorothalonil, dichlofluanid, tolylfluanid, flusulfamide, phthalide, hexachlorobenzene, pencycuron, quintozene;
nitrophenyl derivatives: binapacryl, dinocap, dinobuton;
inorganic active compounds: Bordeaux mixture, copper acetate, copper hydroxide, copper oxychloride, basic copper sulfate, sulfur;
others: spiroxamine, cyflufenamid, cymoxanil, metrafenone.

The present invention furthermore relates to the pharmaceutical use of the compounds of the formula (I) according to the invention and/or the pharmaceutically acceptable salts thereof, in particular their use for treating tumors in mammals such as, for example, humans.

### SYNTHESIS EXAMPLES

The procedures described in the synthesis examples below were used to prepare further compounds I by appropriate modification of the starting compounds. The compounds thus obtained are listed in the table C below, together with physical data.

### Preparation of Malonates VI:

### VI.1 Diethyl 2-(4-methylpyridin-2-yl)malonate

At −60° C., 60 g of a 15% by weight strength solution of n-butyllithium in hexane were added dropwise to a solution of 15 g (0.14 mol) of 2,4-dimethylpyridine in 150 ml of tetrahydrofuran (THF). The mixture was stirred for 15 min and a solution of 16.5 g (0.14 mol) of diethyl carbonate was then added dropwise at −60° C. After 2 h of stirring at this temperature, the reaction mixture was stirred for another 12 h at RT. The reaction mixture was then poured into 300 ml of 10% strength aqueous hydrochloric acid, the ether phase was separated off and the aqueous phase was extracted once with ethyl acetate. The aqueous phase was made alkaline using sodium carbonate solution and extracted three times with dichloromethane. The residue obtained after drying and concentrating the dichloromethane phase is chromatographed on silica gel (cyclohexane/ethyl acetate). This gave 2.3 g of the title compound.

### VI.2 Diethyl 2-(3-methylpyridin-2-yl)malonate

4.7 g (26.2 mmol) of ethyl (3-methylpyridin-2-yl)acetate and 15.5 g (131 mmol) of diethyl carbonate were added to a solution of 1.87 g (27.5 mmol) of sodium ethoxide in 45 ml of toluene. The reaction mixture was heated under reflux for 6 h, and the ethanol formed was distilled off. After cooling, the reaction mixture was poured into water, the pH was adjusted to pH 5-6 using acetic acid, the mixture was extracted three times with ethyl acetate and the organic phase was dried. After concentration of the organic phase, the residue was chromatographed on silica gel. This gave 4.3 g of the title compound.

### VI.3 Diethyl 2-(6-methylpyridin-2-yl)malonate

4.4 g (24.5 mmol) of ethyl (6-methylpyridin-2-yl)acetate and 14.5 g (123 mmol) of diethyl carbonate were added to a solution of 1.87 g (27.5 mmol) of sodium ethoxide in 43 ml of toluene. The reaction mixture was heated at reflux for 6 h, and the ethanol formed was distilled off. After cooling, the reaction mixture was poured into water, the pH was adjusted with acetic acid to pH 5-6, the mixture was extracted three times with ethyl acetate and the organic phase was dried. After concentration of the organic phase, the residue was chromatographed on silica gel. This gave 2.6 g of the title compound.

The malonates of the formula VI listed in table B (malonates VI.4 to VI.64) were prepared in an analogous manner:

TABLE B
Malonates VI

| | | | m/z; |
|---|---|---|---|
| | Het | R | b.p. [° C.] (mm) |
| VI. 1 | 4-methylpyridin-2-yl | C₂H₅ | 252 [M⁺] |
| VI. 2 | 3-methylpyridin-2-yl | C₂H₅ | 252 [M⁺] |
| VI. 3 | 6-methylpyridin-2-yl | C₂H₅ | 252 [M⁺] |
| VI. 4 | 5-methylpyridin-2-yl | C₂H₅ | 252 [M⁺] |
| VI. 5 | 5-ethylpyridin-2-yl | C₂H₅ | 266 [M⁺] |
| VI. 6 | 5-(methoxycarbonyl)pyridin-2-yl | C₂H₅ | |
| VI. 7 | 3,5-difluoropyridin-2-yl | C₂H₅ | 83.86 (0.1) |
| VI. 8 | 3-methylpyridin-4-yl | C₂H₅ | |
| VI. 9 | 3,5-dichloropyridin-2-yl | CH₃ | 278 [M⁺] |
| VI. 10 | 6-chloropyridin-2-yl | C₂H₅ | 272 [M⁺] |
| VI. 11 | 6-methoxypyridazin-3-yl | CH₃ | 241 [M⁺] |
| VI. 12 | 5-NO₂-pyridin-2-yl | | 104° |
| VI. 13 | 3,5-difluoropyridin-2-yl | CH₃ | |
| VI. 14 | 5-CN-pyridin-2-yl | CH₃ | 235 [M⁺] |
| VI. 15 | 5-CN-pyridin-2-yl | C₂H₅ | |
| VI. 16 | pyridin-2-yl | CH₃ | |
| VI. 17 | pyridin-2-yl | C₂H₅ | |
| VI. 18 | 5-(methoxycarbonyl)pyridin-2-yl | CH₃ | 268 [M⁺] |
| VI. 19 | pyridin-3-yl | CH₃ | |
| VI. 20 | pyridin-3-yl | C₂H₅ | |
| VI. 21 | pyridin-4-yl | CH₃ | |
| VI. 22 | pyridin-4-yl | C₂H₅ | |
| VI. 23 | 5-methylpyridin-2-yl | CH₃ | |
| VI. 24 | 4-methylpyridin-2-yl | CH₃ | |
| VI. 25 | 6-methylpyridin-2-yl | CH₃ | |
| VI. 26 | 5-ethylpyridin-2-yl | CH₃ | |
| VI. 27 | 6-chloropyridin-3-yl | C₂H₅ | 272 [M⁺] |
| VI. 28 | 6-chloropyridin-3-yl | CH₃ | |
| VI. 29 | 3-methylpyridin-4-yl | CH₃ | |
| VI. 30 | 3-bromopyridin-4-yl | C₂H₅ | 316 [M⁺] |
| VI. 31 | 3-bromopyridin-4-yl | CH₃ | |
| VI. 32 | 3-methylpyridin-2-yl | CH₃ | |
| VI. 33 | 2-chloropyridin-4-yl | C₂H₅ | 272 [M⁺] |
| VI. 34 | 2-chloropyridin-4-yl | CH₃ | |
| VI. 35 | 2-methylpyridin-4-yl | C₂H₅ | 252 [M⁺] |
| VI. 36 | 2-methylpyridin-4-yl | CH₃ | |
| VI. 37 | 6-chloropyridin-2-yl | CH₃ | |
| VI. 38 | 3-chloropyridin-2-yl | CH₃ | 244 [M⁺] |
| VI. 39 | 3-chloropyridin-2-yl | C₂H₅ | |
| VI. 40 | 4-chloropyridin-2-yl | CH₃ | |
| VI. 41 | 4-chloropyridin-2-yl | C₂H₅ | |
| VI. 42 | 3-ethylpyridin-4-yl | CH₃ | |
| VI. 43 | 3-ethylpyridin-4-yl | C₂H₅ | |
| VI. 44 | 2-chloropyridin-3-yl | CH₃ | |
| VI. 45 | 2-chloropyridin-3-yl | C₂H₅ | |
| VI. 46 | 3-chloropyridin-4-yl | CH₃ | |
| VI. 47 | 3-chloropyridin-4-yl | C₂H₅ | |
| VI. 48 | 3-chloro-5-cyanopyridin-2-yl | CH₃ | 245 [M⁺] |
| VI. 49 | 3-chloro-5-cyanopyridin-2-yl | C₂H₅ | 245 [M⁺] |
| VI. 50 | 4,6-dichloropyridin-3-yl | C₂H₅ | 306 [M⁺] |
| VI. 51 | 4,6-dichloropyridin-3-yl | CH₃ | |
| VI. 52 | 3,5-dichloropyridin-2-yl | C₂H₅ | |
| VI. 53 | 3-ethoxypyridin-2-yl | C₂H₅ | 282 [M⁺] |
| VI. 54 | 3-ethoxypyridin-2-yl | CH₃ | |
| VI. 55 | 3,5-dibromopyridin-2-yl | CH₃ | 368 [M⁺] |
| VI. 56 | 3,5-dibromopyridin-2-yl | C₂H₅ | |
| VI. 57 | 3,5-dichloropyridin-4-yl | C₂H₅ | 306 [M⁺] |
| VI. 58 | 3,5-dichloropyridin-4-yl | CH₃ | |
| VI. 59 | 2-methylpyridin-3-yl | CH₃ | |
| VI. 60 | 2-methylpyridin-3-yl | C₂H₅ | |
| VI. 61 | 2,4-dichloro-6-methylpyridin-3-yl | C₂H₅ | 320 [M⁺] |
| VI. 62 | 2,4-dichloro-6-methylpyridin-3-yl | CH₃ | |
| VI. 63 | 3-iodopyridin-2-yl | C₂H₅ | 364 [M⁺] |
| VI. 64 | 3-iodopyridin-2-yl | CH₃ | |

b.p. boiling point

### Preparation of the Compounds I, II and III

### Example 1

### 5-Chloro-7-(4-methylpiperidin-1-yl)-6-pyrazin-2-yl-[1,2,4]triazolo[1,5-a]pyrimidine

### 1.1 6-Pyrazin-2-yl-[1,2,4]triazolo[1,5a]pyrimidine-5,7-diol

1 g (4.2 mmol) of dimethyl 2-pyrazin-2-ylmalonate and 0.37 mmol of 3-amino-1,2,4-triazole in 1.2 ml of tributylamine were heated at 150° C. for 3 h, and the methanol formed was distilled off. After cooling, the reaction mixture was taken up in 15 ml of 3% strength aqueous sodium hydroxide solution and extracted twice with methyl tert-butyl ether. The aqueous phase was acidified with 10% strength HCl, and the precipitated solid was filtered off. This gave 0.7 g of the title compound. (72%)

### 1.2 5,7-Dichloro-6-pyrazin-2-yl[1,2,4]triazolo[1,5-a]pyrimidine

0.5 g (2.2 mmol) of 6-pyrazin-2-yl-[1,2,4]triazolo[1,5-a]pyrimidine-5,7-diol and 0.52 g (5.43 mmol) of trimethylamine hydrochloride in 5 ml of phosphoryl chloride were heated at reflux for 6 h. The reaction mixture was carefully poured into ice-water, neutralized with sodium carbonate and extracted 3 times with ethyl acetate. Concentration gave 0.07 g (12%) of the title compound.

### 1.3 5-Chloro-7-(4-methylpiperidin-1-yl)-6-pyrazin-2-yl-[1,2,4]triazolo[1,5-a]pyrimidine

0.07 g (0.26 mmol) of 5,7-dichloro-6-pyrazin-2-yl-[1,2,4]triazolo[1,5-a]pyrimidine and 0.026 g (0.26 mmol) of 4-methylpiperidine in 3 ml of dichloromethane were stirred at room temperature until the conversion of the starting material was complete. The mixture was taken up in dichloromethane, the solution was washed in each case once with water and 10% strength hydrochloric acid and the organic phase was separated off. Drying and concentration of the organic phase gave 0.04 g (46%) of the title compound (m.p. 167° C.).

### Example 2

### R-5-Chloro-7-(1,2-dimethylpropylamino)-6-(5-nitropyridin-2-yl)-[1,2,4]triazolo-[1,5-a]pyrimidine

### 2.1 6-(5-Nitropyridin-2-yl)-[1,2,4]triazolo[1,5-a]pyrimidine-5,7-diol

1.13 g (4.46 mmol) of dimethyl 2-(5-nitropyridin-2-yl)malonate and 0.3 g (3.57 mmol) of 3-amino-1,2,4-triazole in 0.73 g of tributylamine were heated at 150° C. for 12 h, and the methanol formed was distilled off. After cooling, the reaction mixture was taken up in 50 ml of 10% strength aqueous sodium hydroxide solution, and the precipitated solid was filtered off. The residue obtained was used for the next reaction without further purification.

### 2.2 5,7-Dichloro-6-(5-nitropyridin-2-yl)-[1,2,4]triazolo[1,5a]pyrimidine

0.5 g (1.82 mmol) of 6-(5-nitropyridin-2-yl)-[1,2,4]triazolo[1,5-a]pyrimidine-5,7-diol and 0.54 g (4.56 mmol) of trimethylamine hydrochloride in 7 ml of phosphoryl chloride were heated at reflux for 4.5 h. The reaction mixture was carefully added to ice-water, neutralized with sodium bicarbonate and extracted 4 times with ethyl acetate. Concentration gave 0.26 g of the title compound.

### 2.3 R-5-Chloro-7-(1,2-dimethylpropylamino)-6-(5-nitropyridin-2-yl)-[1,2,4]triazolo-[1,5-a]pyrimidine

0.1 g (0.34 mmol) of 5,7-dichloro-6-(5-nitropyridin-2-yl)-[1,2,4]triazolo-[1,5a]pyrimidine and 0.03 g (0.34 mmol) of (R)-3-methyl-2-butylamine in 5 ml of dichloromethane were stirred until the conversion of the starting material was complete. The reaction mixture was taken up in dichloromethane and washed with water and 5% strength hydrochloric acid, and the organic phase was separated off. Drying and concentration of the organic phase gave 56 mg of the title compound.

### Example 3

### R-5-Chloro-7-(1,2-dimethylpropylamino)-6-(4-methylpyridin-2-yl)-[1,2,4]triazolo[1,5-a]pyrimidine

### 3.1 6-(4-Methylpyridin-2-yl)-[1,2,4]triazolo[1,5-a]pyrimidine-5,7-dione

4 g (16 mmol) of diethyl 2-(4-methylpyridin-2-yl)malonate and 1.47 g (17.5 mmol) of 3-amino-1,2,4-triazole in 3 g of n-tributylamine were heated at 145° C. for 10 hours, and the ethanol formed was distilled off. After cooling, the reaction mixture was taken up in 40 ml of 4% strength aqueous sodium hydroxide solution, the solution was extracted with ethyl acetate and the aqueous solution was acidified with 10% strength aqueous hydrochloric acid. The resulting precipitate was separated off and, after drying (2.8 g), used without further purification for the next step.

### 3.2 5,7-Dichloro-6-(4-methylpyridin-2-yl)-[1,2,4]triazolo[1,5-a]pyrimidine

1 g (4.1 mmol) of 6-(4-methylpyridin-2-yl)-4H-[1,2,4]triazolo[1,5-a]pyrimidine-5,7-dione was initially charged in 7 ml of phosphoryl chloride. 1 g of trimethylamine hydrochloride was added, and the mixture was heated at reflux for 7 hours. After cooling, the reaction mixture was carefully poured into ice-water and extracted with ethyl acetate, and the organic phase was, after drying, concentrated. This gave 0.18 g of the title compound.

### 3.3 R-[5-Chloro-6-(4-methylpyridin-2-yl)-[1,2,4]triazolo[1,5-a]pyrimidin-7-yl]-(1,2-dimethylpropyl)amine

0.18 g (0.64 mmol) of 5,7-dichloro-6-(4-methylpyridin-2-yl)-[1,2,4]triazolo[1,5-a]pyrimidine, 0.05 g (0.6 mmol) of R-2-methyl-3-butylamine and 0.1 ml of triethylamine in 1.5 ml of dichloromethane were stirred at RT for 12 h. The reaction mixture was then diluted with dichloromethane and taken up in water, and the aqueous phase was extracted with ethyl acetate. The combined organic phases were dried and concentrated. The residue was separated by HPLC (RP-18 column, 40° C., acetonitrile with 0.1% by volume of a trifluoroacetic acid/water mixture (5:95-95:5) and 0.1% by volume of trifluoroacetic acid), which gave 35 mg of the title compound (m/z: 331 [M⁺])

### Example 4

### 5-Chloro-7-(4-methylpiperidin-1-yl)-6-(3-methylpyridin-2-yl)-[1,2,4]triazolo-[1,5-a]pyrimidine

### 4.1 6-(3-Methylpyridin-2-yl)-[1,2,4]triazolo[1,5-a]pyrimidine-5,7-dione

4.15 g (16.5 mmol) of diethyl 2-(3-methylpyridin-2-yl)malonate and 1.53 g (18.2 mmol) of 3-amino-1,2,4-triazole in 3.1 g of n-tributylamine were heated at 150° C. for 10 hours, and the ethanol formed was distilled off. After cooling, the reaction mixture was taken up in 20 ml of 10% strength aqueous sodium hydroxide solution and extracted with methyl tert-butyl ether, and the aqueous solution was acidified with 10% strength aqueous hydrochloric acid. The precipitate formed was separated off and, after drying (2.0 g), used without further purification for the next step.

### 4.2 5,7-Dichloro-6-(3-methylpyridin-2-yl)-[1,2,4]triazolo[1,5-a]pyrimidine

1 g (4.1 mmol) of 6-(3-methylpyridin-2-yl)-4H-[1,2,4]triazolo[1,5-a]pyrimidine-5,7-dione was initially charged in 8 ml of phosphoryl chloride. 1 g of trimethylamine hydrochloride was added, and the mixture was heated at reflux for 8 hours. After cooling, the reaction mixture was carefully poured into ice-water and extracted with ethyl acetate, and the organic phase was, after drying, concentrated. This gave 0.46 g of the title compound.

### 4.3 5-Chloro-7-(4-methylpiperidin-1-yl)-6-(3-methylpyridin-2-yl)-[1,2,4]triazolo-[1,5-a]pyrimidine

0.1 g (0.36 mmol) of 5,7-dichloro-6-(3-methylpyridin-2-yl)-[1,2,4]triazolo-[1,5-a]pyrimidine, 0.032 g (0.32 mmol) of 4-methylpiperidine and 0.05 ml of triethylamine in 2 ml of dichloromethane were stirred at RT for 12 h. The mixture was then diluted with dichloromethane and taken up in water, and the aqueous phase was extracted with ethyl acetate. The combined organic phases were dried and concentrated. The residue was chromatographed on a prefilled syringe column on Chromabond SiOH. This gave 13 mg of the title compound.

### Example 5

### 5-Chloro-7-(4-methylpiperidin-1-yl)-6-(6-methylpyridin-2-yl)-[1,2,4]triazolo-[1,5-a]pyrimidine

### 5.1 6-(6-Methylpyridin-2-yl)-[1,2,4]triazolo[1,5-a]pyrimidine-5,7-dione

3.1 g (12.34 mmol) of diethyl 2-(6-methylpyridin-2-yl)malonate and 1.14 g (13.6 mmol) of 3-amino-1,2,4-triazole in 2.33 g of n-tributylamine were heated at 150° C. for 14 hours, and the ethanol formed was distilled off. After cooling, the reaction mixture was taken up in 20 ml of 10% strength aqueous sodium hydroxide solution and extracted with methyl tert-butyl ether, and the aqueous solution was acidified with 10% strength aqueous hydrochloric acid. The precipitate formed was separated off and, after drying (1.9 g), used without further purification for the next step.

### 5.2 5,7-Dichloro-6-(6-methylpyridin-2-yl)-[1,2,4]triazolo[1,5-a]pyrimidine

0.8 g (3.3 mmol) of 6-(6-methylpyridin-2-yl)-4H-[1,2,4]triazolo[1,5-a]pyrimidine-5,7-dione was initially charged in 7 ml of phosphoryl chloride. 0.8 g of trimethylamine hydrochloride was added, and the mixture was heated at reflux for 5 hours. After cooling, the reaction mixture was carefully poured into ice-water and extracted with ethyl acetate, and the organic phase was, after drying, concentrated. This gave 0.2 g of the title compound.

### 5.3 5-Chloro-7-(4-methylpiperidin-1-yl)-6-(6-methylpyridin-2-yl)-[1,2,4]triazolo-[1,5-a]pyrimidine

0.2 g (0.71 mmol) of 5,7-dichloro-6-(3-methylpyridin-2-yl)-[1,2,4]triazolo-[1,5-a]pyrimidine, 0.064 g (0.64 mmol) of 4-methylpiperidine and 0.072 ml of triethylamine in 2 ml of dichloromethane were stirred at room temperature for 12 h. The mixture was then diluted with dichloromethane and taken up in water, and the aqueous phase was extracted with ethyl acetate. The combined organic phases were dried and concentrated. The residue was chromatographed on a pre-filled syringe column on Chromabond SiOH. This gives 0.1 g of the title compound. M.p. 128° C.

The compounds of the general formulae I, II and III where Y=H listed in tables C, D and E below were prepared according to the given procedures.

All products were characterized by HPLC, mass spectrometry, by combined HPLC/mass spectrometry (High Performance Liquid Chromatography Mass Spectrometry) or by their melting point. For HPLC, an analytical RP-18 column (Chromolith Speed ROD from Merck KGaA, Germany), which was operated at 40° C., was used. The mobile phase used was acetonitrile with 0.1% by volume of a trifluoroacetic acid water mixture and 0.1% by volume of trifluoroacetic acid. (The ratio of trifluoroacetic acid/water was changed from 5:95 to 95:5 over a period of 5 minutes). Mass spectrometry was carried out using a quadrupole mass spectrometer with electrospray ionization at 80 V in the positive mode.

TABLE C
Compounds of the formula I where Y = H

| | | | | m.p.¹⁾, m/z²⁾, |
|---|---|---|---|---|
| Ex. | NR¹R² | X | Het | HPLC³⁾ |
| 5 | 4-methylpiperidin-1-yl | Cl | 5-NO₂-pyridin-2-yl | 374 [M⁺] |
| 6 | 4-methylpiperidin-1-yl | 4-methyl- | 5-NO₂-pyridin-2-yl | 75° C. |
| | | piperidin-1-yl | | |
| 7 | R-3-methylbutan-2-yl- | Cl | 5-NO₂-pyridin-2-yl | 362 [M⁺] |
| | amino | | | |
| 8 | 4-methylpiperidin-1-yl | Cl | 3,5-difluoropyridin-2- | 203° C. |
| | | | yl | |
| 9 | R-3-methylbutan-2-yl- | Cl | 3,5-difluoropyridin-2- | 148° C. |
| | amino | | yl | |
| 10 | 4-methylpiperidin-1-yl | Cl | 5-(CO₂CH₃)- | 387 [M⁺] |
| | | | pyridin-2-yl | |
| 11 | 4-methylpiperidin-1-yl | Cl | pyridin-2-yl | 329 [M⁺] |
| 12 | R-3-methylbutan-2-ylamino | Cl | pyridin-2-yl | 317 [M⁺] |
| 13 | dimethylamino | Cl | 5-(CO₂CH₃)- | 332 [M⁺] |
| | | | pyridin-2-yl | |
| 14 | 4-methylpiperidin-1-yl | Cl | pyridin-3-yl | 144° C. |
| 15 | 4-methylpiperidin-1-yl | Cl | 5-CN-pyridin-2-yl | 354 [M⁺] |
| 16 | R-3-methylbutan-2-ylamino | Cl | pyridin-3-yl | 119° C. |
| 17 | 4-methylpiperidin-1-yl | Cl | pyridin-4-yl | 161° C. |
| 18 | R-3-methylbutan-2-ylamino | Cl | pyridin-4-yl | 317 [M⁺] |
| 19 | 4-methylpiperidin-1-yl | Cl | 5-CH₃-pyridin-2-yl | 141° C. |
| 20 | R-3-methylbutan-2-ylamino | Cl | 5-CH₃-pyridin-2-yl | 331 [M⁺] |
| 21 | N-methyl-N-(2-methyl- | Cl | 5-CH₃-pyridin-2-yl | 151° C. |
| | propan-1-yl)amino | | | |
| 22 | R-3-methylbutan-2-ylamino | Cl | 6-CH₃-pyridin-2-yl | 331 [M⁺] |
| 23 | 4-methylpiperidin-1-yl | OH | 5-CH₃-NH-pyridin- | 339 [M⁺] |
| | | | 2-yl | |
| 24 | 2-methylpiperidin-1-yl | Cl | 5-CH₃-pyridin-2-yl | 343 [M⁺] |
| 25 | benzylamino | Cl | 5-CH₃-pyridin-2-yl | 351 [M⁺] |
| 26 | R-3,3-dimethylbutan-2- | Cl | 5-CH₃-pyridin-2-yl | 345 [M⁺] |
| | ylamino | | | |
| 27 | 4-methylpiperidin-1-yl | Cl | 5-C₂H₅-pyridin-2-yl | 357 [M⁺] |
| 28 | 4-methylpiperidin-1-yl | Cl | 3,5-dichloropyridin-2- | 398 [M⁺] |
| | | | yl | |
| 29 | R-3-methylbutan-2-ylamino | Cl | 3,5-dichloropyridin- | 386 [M⁺]; |
| | | | 2-yl | isomer A |
| 30 | R-3-methylbutan-2-ylamino | Cl | 3,5-dichloropyridin-2- | 386 [M⁺]; |
| | | | yl | isomer B |
| 31 | R-3-methylbutan-2-ylamino | Cl | 3,5-dichloropyridin-2- | 78° C.; isomer A |
| | | | yl | and isomer B |
| 32 | 4-methylpiperidin-1-yl | Cl | pyrazin-2-yl | 167° C. |
| 33 | 4-methylpiperidin-1-yl | 4-methyl- | pyrazin-2-yl | 393 [M⁺] |
| | | piperidin-1-yl | | |
| 34 | 4-methylpiperidin-1-yl | Cl | 6-chloropyridazin-3-yl | 252° C. |
| 35 | 4-methylpiperidin-1-yl | Cl | 6-methoxypyridazin- | |
| | | | 3-yl | |
| 36 | N-methyl-N-(2-methyl- | Cl | 4-methylpyridin-2-yl | 331 [M⁺] |
| | propan-1-yl)amino | | | |
| 37 | 4-methylpiperidin-1-yl | Cl | 6-chloropyridin-3-yl | 206° C. |
| 38 | R-3-methylbutan-2-ylamino | Cl | 6-chloropyridin-3-yl | 135° C. |
| 39 | 4-methylpiperidin-1-yl | Cl | 3-methylpyridin-4-yl | 168° C. |
| 40 | R-3-methylbutan-2-ylamino | Cl | 3-methylpyridin-4-yl | 167° C. |
| 41 | 4-methylpiperidin-1-yl | Cl | 3-bromopyridin-4-yl | 188° C. |
| 42 | R-3-methylbutan-2-ylamino | Cl | 3-bromopyridin-4-yl | 127° C.; |
| | | | | isomer A |
| 43 | R-3-methylbutan-2-ylamino | Cl | 3-bromopyridin-4-yl | 114° C.; |
| | | | | isomer B; |
| 44 | R-3-methylbutan-2-ylamino | Cl | 3-methylpyridin-2-yl | 331 [M⁺] |
| 45 | R-3-methylbutan-2-ylamino | Cl | 4-chloropyridin-2-yl | 351 [M⁺] |
| 46 | 4-methylpiperidin-1-yl | Cl | 2-CH₃-pyridin-4-yl | 149° C. |
| 47 | 4-methylpiperidin-1-yl | Cl | 3-chloropyridin-2-yl | 141° C. |
| 48 | R-3-methylbutan-2-ylamino | Cl | 6-chloropyridin-2-yl | 124° C. |
| 49 | 4-methylpiperidin-1-yl | Cl | 6-chloropyridin-2-yl | 82° C. |
| 50 | R-3-methylbutan-2-ylamino | Cl | 3-chloropyridin-2-yl | 191° C. |
| | | | | isomer A |
| 51 | R-3-methylbutan-2-yl-amino | Cl | 3-chloropyridin-2-yl | 190° C.; |
| | | | | isomer A:isomer B = 35:65 |
| 52 | R-3-methylbutan-2-ylamino | Cl | 3-chloropyridin-2-yl | 187° C.; |
| | | | | isomer B |
| 53 | 4-methylpiperidin-1-yl | Cl | 2-chloropyridin-4-yl | |
| 54 | R-3-methylbutan-2-ylamino | Cl | 2-chloropyridin-4-yl | |
| 55 | 4-methylpiperidin-1-yl | Cl | 4-chloropyridin-2-yl | 364 [M⁺] |
| 56 | 4-methylpiperidin-1-yl | Cl | 3-ethylpyridin-4-yl | 167° C. |
| 57 | 4-methylpiperidin-1-yl | OCH₃ | 5-CH₃-pyridin-2-yl | 339 [M⁺] |
| 58 | N-ethyl-N-2-CH₃-prop-2-en- | Cl | 3,5-dichloropyridin-2- | |
| | amino | | yl | |
| 59 | R-3-methylbutan-2-ylamino | Cl | 2-methylpyridin-4-yl | 161° C. |
| 60 | 4-methylpiperidin-1-yl | Cl | 2-chloropyridin-3-yl | 200° C. |
| 61 | R-3-methylbutan-2-ylamino | Cl | 2-chloropyridin-3-yl | 186° C. |
| 62 | 4-methylpiperidin-1-yl | Cl | 3-chloropyridin-4-yl | 208° C. |
| 63 | 4-methylpiperidin-1-yl | OCH₃ | 3-chloropyridin-4-yl | 218° C. |
| 64 | R-3-methylbutan-2-ylamino | Cl | 3-chloropyridin-4-yl | 140° C. |
| 65 | Benzylamino | Cl | 3,5-dichloropyridin-2- | 228° C. |
| | | | yl | |
| 66 | R-3,3-dimethylbutan-2- | Cl | 3,5-dichloropyridin-2- | 165° C. |
| | ylamino | | yl | isomer mixture |
| | | | | A:B 60:40 |
| 67 | R-3,3-dimethylbutan-2- | Cl | 3,5-dichloropyridin-2- | 140° C. |
| | ylamino | | yl | isomer mixture |
| | | | | A:B 20:80 |
| 68 | 4-methylpiperidin-1-yl | Cl | 3,5-dichloropyridin-4- | 159° C. |
| | | | yl | |
| 69 | 4-methylpiperidin-1-yl | OCH₃ | 3,5-dichloropyridin-2- | 170° C. |
| | | | yl | |
| 70 | 4-methylpiperidin-1-yl | CH(COOCH₃)₂ | 3,5-dichloropyridin-2- | 95° C. |
| | | | yl | |
| 71 | 4-methylpiperidin-1-yl | CH₃ | 3,5-dichloropyridin-2- | 162° C. |
| | | | yl | |
| 72 | R-3-methylbutan-2-ylamino | Cl | 3,5-dichloropyridin-4- | 146° C. |
| | | | yl | |
| 73 | 2-methylpyrrolidin-1-yl | Cl | 3,5-dichloropyridin-2- | 81° C.; |
| | | | yl | isomer mixture |
| | | | | A:B 60:40 |
| 74 | S-3,3-dimethylbutan-2- | Cl | 3,5-dichloropyridin-2- | 216° C. |
| | ylamino | | yl | |
| 75 | S-3,3-dimethylbutan-2- | Cl | 3,5-dichloropyridin-2- | 151° C.; |
| | ylamino | | yl | isomer mixture |
| | | | | A:B 1:2 |
| 76 | S-3-methylbutan-2-yl-amino | Cl | 3,5-dichloropyridin-2- | 386 [M⁺]; |
| | | | yl | isomer A |
| 77 | S-3-methylbutan-2-yl-amino | Cl | 3,5-dichloropyridin-2- | isomer mixture |
| | | | yl | A:B 1:3 |
| 78 | N-methyl-N-(2- | Cl | 3,5-dichloropyridin-2- | 167° C. |
| | methylpropan-1-yl)amino | | yl | |
| 79 | 4-methylpiperidin-1-yl | Cl | 3-chloro-5- | 388 [M⁺] |
| | | | cyanopyridin-2-yl | |
| 80 | R-3-methylbutan-2-ylamino | Cl | 3-chloro-5- | 91° C. |
| | | | cyanopyridin-2-yl | |
| 81 | 4-methylpiperidin-1-yl | CN | 3,5-dichloropyridin-2- | 123° C. |
| | | | yl | |
| 82 | 4-methylpiperidin-1-yl | Cl | 3-ethoxypyridin-2-yl | 373 [M⁺] |
| 83 | 4-methylpiperidin-1-yl | Cl | 4,6-dichloropyridin-3- | |
| | | | yl | |
| 84 | 4-methylpiperidin-1-yl | 4-methyl- | 3,5-dichloropyridin-2- | 460 [M⁺] |
| | | piperidin-1-yl | yl | |
| 85 | 4-methylpiperidin-1-yl | Cl | 3-Cl-5-CONH₂- | 407 [M⁺] |
| | | | pyridin-2-yl | |
| 86 | R,S-3-methylbutan-2-yl- | Cl | 3,5-dichloropyridin-2- | 62° C. |
| | amino | | yl | |
| 87 | R-3-methylbutan-2-ylamino | Cl | 3-ethoxypyridin-2-yl | |
| 88 | 4-methylpiperidin-1-yl | O—CH₃ | 3,5-dichloropyridin-4- | 150° C. |
| | | | yl | |
| 89 | R-3-methylbutan-2-ylamino | Cl | 3,5-dibromopyridin-2- | 475 [M⁺] |
| | | | yl | |
| 90 | 4-methylpiperidin-1-yl | Cl | 3,5-dibromopyridin-2- | 487 [M⁺] |
| | | | yl | |
| 91 | 4-methylpiperidin-1-yl | CH₃ | 3,5-dichloropyridin-4- | 149° C. |
| | | | yl | |
| 92 | 4-methylpiperidin-1-yl | CH(COOCH₃)₂ | 3,5-dichloropyridin-4- | 216° C. |
| | | | yl | |
| 93 | 4-methylpiperidin-1-yl | CH(COOCH₃)₂ | 3-bromopyridin-4-yl | 196° C. |
| 94 | 4-methylpiperidin-1-yl | CH₃ | 3-bromopyridin-4-yl | 208° C. |
| 95 | 4-methylpiperidin-1-yl | —S—CH₃ | 3,5-dichloropyridin-2- | 165° C. |
| | | | yl | |
| 96 | 4-methylpiperidin-1-yl | —S—CH₃ | 3-CH₃S-5-Cl-pyridin- | 85° C. |
| | | | 2-yl | |
| 97 | 4-methylpiperidin-1-yl | —SO—CH₃ | 3,5-dichloropyridin-2- | 110° C. |
| | | | yl | |
| 98 | 4-methylpiperidin-1-yl | —SO₂—CH₃ | 3,5-dichloropyridin-2- | 239° C. |
| | | | yl | |
| 99 | 4-methylpiperidin-1-yl | Cl | 2-methylpyridin-3-yl | 204° C. |
| 100 | R-3-methylbutan-2-ylamino | Cl | 2-methylpyridin-3-yl | 165° C. |
| 101 | 4-methylpiperidin-1-yl | Cl | 2,4-dichloro-6- | 169° C. |
| | | | methylpyridin-3-yl | |
| 102 | 4-methylpiperidin-1-yl | Cl | 3-I-pyridin-2-yl | 455 [M⁺] |
| 103 | R-3-methylbutan-2-ylamino | Cl | 3-I-pyridin-2-yl | |
| 104 | 4-methylpiperidin-1-yl | Cl | 4-Cl-pyridin-3-yl | M⁺ 363 |

¹⁾melting point in ° C.
²⁾m/z of the M⁺ or the [M + H]⁺ peak
³⁾HPLC analysis retention time in minutes

TABLE D
Compounds of the formula II (Y = H)

| | Het | m.p.¹⁾, m/z²⁾, HPLC³⁾ |
|---|---|---|
| II-1 | pyrazin-2-yl | |
| II-2 | 5-NO₂-pyridin-2-yl | M⁺ 275 |
| II-3 | 4-methylpyrid-2-yl | |
| II-4 | 3-methylpyrid-2-yl | |
| II-5 | 6-methyl-2-yl | |
| II-6 | 3,5-difluoropyridin-2-yl | |
| II-7 | 5-(CO₂CH₃)-pyridin-2-yl | M⁺ 287 |
| II-8 | pyridin-2-yl | |
| II-9 | pyridin-3-yl | |
| II-10 | 5-CN-pyridin-2-yl | M⁺ 253 |
| II-11 | pyridin-4-yl | |
| II-12 | 5-CH₃-pyridin-2-yl | M⁺ 243 |
| II-13 | 5-C₂H₅-pyridin-2-yl | |
| II-14 | 3,5-dichloropyridin-2-yl | M⁺ 298 |
| II-15 | 6-chloropyridazin-3-yl | |
| II-16 | 6-methoxypyridazin-3-yl | |
| II-17 | 6-Cl-pyridin-3-yl | |
| II-18 | 3-CH₃-pyridin-4-yl | M⁺ 244 |
| II-19 | 3-Br-pyridin-4-yl | decomp. >290° C.; |
| | | M⁺ 308 |
| II-20 | 2-CH₃-pyridin-4-yl | |
| II-21 | 6-Cl-pyridin-2-yl | |
| II-22 | 3-Cl-pyridin-2-yl | |
| II-23 | 2-Cl-pyridin-4-yl | |
| II-24 | 4-Cl-pyridin-2-yl | |
| II-25 | 3-C₂H₅-pyridin-4-yl | |
| II-26 | 2-Cl-pyridin-3-yl | M⁺ 264 |
| II-27 | 3-Cl-pyridin-4-yl | |
| II-28 | 3-chloro-5-cyanopyridin-2-yl | |
| II-29 | 4,6-dichloropyridin-3-yl | M⁺ 298 |
| II-30 | 3-ethoxypyridin-2-yl | |
| II-31 | 3,5-dibromopyridin-2-yl | M⁺ 387 |
| II-32 | 3,5-dichloropyridin-4-yl | decomp. 110° |
| II-33 | 2-CH₃-pyridin-3-yl | |
| II-34 | 2,4-dichloro-6-methylpyridin-3-yl | M⁺ 312 |
| II-35 | 3-iodopyridin-2-yl | M⁺ 356 |

¹⁾melting point in ° C.
²⁾m/z of the M⁺ or the [M + H]⁺ peak
³⁾HPLC analysis retention time in minutes

TABLE E
Compounds of the formula III (Y = H, Hal = Cl)

| | Het | m.p.¹⁾, m/z²⁾, HPLC³⁾ |
|---|---|---|
| III-1 | pyrazin-2-yl | |
| III-2 | 5-NO₂-pyridin-2-yl | M⁺ 311 |
| III-3 | 4-CH₃-pyridin-2-yl | |
| III-4 | 3-CH₃-pyridin-2-yl | |
| III-5 | 6-methylpyridin-2-yl | |
| III-6 | 3,5-difluoropyridin-2-yl | |
| III-7 | 5-(CO₂CH₃)-pyridin-2-yl | |
| III-8 | pyridin-2-yl | |
| III-9 | pyridin-3-yl | |
| III-10 | 5-CN-pyridin-2-yl | M⁺ 291 |
| III-11 | pyridin-4-yl | |
| III-12 | 5-CH₃-pyridin-2-yl | M⁺ 280 |
| III-13 | 5-C₂H₅-pyridin-2-yl | |
| III-14 | 3,5-dichloropyridin-2-yl | M⁺ 335 |
| III-15 | 6-chloropyridazin-3-yl | |
| III-16 | 6-methoxypyridazin-3-yl | |
| III-17 | 6-Cl-pyridin-3-yl | |
| III-18 | 3-CH₃-pyridin-4-yl | M⁺ 280 |
| III-19 | 3-Br-pyridin-4-yl | M⁺ 346 |
| III-20 | 2-Cl-pyridin-4-yl | M⁺ 300 |
| III-21 | 2-CH₃-pyridin-4-yl | |
| III-22 | 6-Cl-pyridin-2-yl | |
| III-23 | 3-Cl-pyridin-2-yl | |
| III-24 | 4-Cl-pyridin-2-yl | |
| III-25 | 3-C₂H₅-pyridin-4-yl | |
| III-26 | 2-Cl-pyridin-3-yl | |
| III-27 | 3-Cl-pyridin-4-yl | M⁺ 302 |
| III-28 | 3-chloro-5-cyanopyridin-2-yl | M⁺ 327 |
| III-29 | 4,6-dichloropyridin-3-yl | M⁺ 336 |
| III-30 | 3-ethoxypyridin-2-yl | M⁺ 310 |
| III-31 | 3,5-dibromopyridin-2-yl | M⁺ 453 |
| III-32 | 3,5-dichloropyridin-4-yl | M⁺ 336 |
| III-33 | 2-CH₃-pyridin-3-yl | M⁺ 280 |
| III-34 | 2,4-dichloro-6-methylpyridin-3-yl | M⁺ 350 |
| III-35 | 3-iodopyridin-2-yl | M⁺ 392 |

¹⁾melting point in ° C.
²⁾m/z of the M⁺ or the [M + H]⁺ peak
³⁾HPLC analysis retention time in minutes

### Examples of the Action Against Harmful Fungi

The fungicidal action of the compounds of formula I was demonstrated by the following tests:

The respective active compound was prepared as a stock solution with 25 mg of active compound which was made up to 10 ml with a mixture of acetone and/or dimethyl sulfoxide (DMSO) and the emulsifier Uniperol® EL (wetting agent having emulsifying and dispersing action based on ethoxylated alkylphenols) in a volume ratio solvent/emulsifier of 99 to 1. The solution was then made up to 100 ml with water. This stock solution was diluted to the active compound concentration stated below using the solvent/emulsifier/water mixture described.

### Use Example 1

### Activity Against Early Blight of Tomato Caused by Alternaria solani

Leaves of potted plants of the cultivar “Goldene Konigin” were sprayed to runoff point with an aqueous suspension having the concentration of active compounds stated below. The next day, the leaves were infected with an aqueous spore suspension of Alternaria solani in 2% biomalt solution having a density of 0.17×10⁶ spores/ml. The plants were then placed in a water-vapor-saturated chamber at temperatures of between 20 and 22° C.

After 5 days, the infection on the untreated, but infected control plants had developed to such an extent that the infection could be determined visually in %.

In this test, the plants which had been treated with 250 ppm (concentration of active compound) of the compound from example 1, 8, 9, 21, 26, 28, 38, 30, 31, 43, 47, 51, 52, 58, 60, 61, 62, 66, 67, 68, 69, 71, 72, 73, 74, 76, 77, 75, 78, 79, 80, 81, 83, 86, 88, 89, 90, 91, 95, 96, 97, 101 showed an infection of at most 30%, whereas the untreated plants were 90% infected.

### Use Example 2

### Activity Against Gray Mold on Bell Pepper Leaves Caused by Botrytis cinerea, Protective Application

Bell pepper seedlings of the cultivar “Neusiedler Ideal Elite” were, after 2-3 leaves were well developed, sprayed to runoff point with an aqueous suspension having the concentration of active compound stated below. The next day, the treated plants were inoculated with a spore suspension of Botrytis cinerea which contained 1.7×10⁶ spores/ml in a 2% strength aqueous biomalt solution. The test plants were then placed in a dark climatized chamber at 22-24° C. and high atmospheric humidity. After 5 days, the extent of the fungal infection on the leaves could be determined visually in %.

In this test, the plants which had been treated with 250 ppm of the compound from example 3, 5, 21, 22, 28, 30, 31, 43, 47, 51, 52, 56, 58, 66, 67, 71, 72, 73, 76, 78, 79, 81, 83, 86, 88, 89, 90, 91, 95, 96, 97 or 101 showed an infection of at most 20%, whereas the untreated plants were 70% infected.

### Use Example 3

### Activity Against Net Blotch of Barley Caused by Pyrenophora teres, 1 Day Protective Application

Leaves of potted barley seedlings of the cultivar “Hanna” were sprayed to runoff point with an aqueous suspension having the concentration of active compound stated below. 24 h after the spray coating had dried on, the test plants were inoculated with an aqueous spore suspension of Pyrenophora [syn. Drechslera] teres, the net blotch pathogen. The test plants were then placed in a greenhouse at temperatures of from 20 to 24° C. and 95 to 100% relative atmospheric humidity. After 6 days, the extent of the fungal infection on the leaves was determined visually in %.

In this test, the plants which had been treated with 250 ppm of the compound from example 2, 5, 8, 19, 26, 41, 46, 50, 62, 64, 69, 74 or 80 showed an infection of at most 20%, whereas the untreated plants were 90% infected.

### Use Example 4

### Curative Activity Against Brown Rust of Wheat Caused by Puccinia recondita

Leaves of potted wheat seedlings of the cultivar “Kanzler” were inoculated with a spore suspension of brown rust (Puccinia recondita). The pots were then placed in a chamber with high atmospheric humidity (90 to 95%) and 20 to 22° C. for 24 hours. During this time, the spores germinated and the germ tubes penetrated into the leaf tissue. The next day, the infected plants were sprayed to runoff point with the active compound solution described above at the active compound concentration stated below. After the spray coating had dried on, the test plants were cultivated in a greenhouse at temperatures between 20 and 22° C. and 65 to 70% relative atmospheric humidity for 7 days. The extent of the rust fungus development on the leaves was then determined.

In this test, the plants which had been treated with 250 ppm of the compound from example 40, 60 or 64 showed an infection of at most 5%, whereas the untreated plants were 90% infected.

### Use Example 5

### Protective Activity Against Puccinia recondita on Wheat (Brown Rust of Wheat)

Leaves of potted wheat seedlings of the cultivar “Kanzler” were sprayed to runoff point with an aqueous suspension having the active compound concentration stated below. The next day, the treated plants were inoculated with a spore suspension of brown rust of wheat (Puccinia recondita). The plants were then placed in a chamber with high atmospheric humidity (90 to 95%) at 20 to 22° C. for 24 hours. During this time, the spores germinated and the germ tubes penetrated into the leaf tissue. The next day, the test plants were returned to the greenhouse and cultivated at temperatures between 20 and 22° C. and 65 to 70% relative atmospheric humidity for a further 7 days. The extent of the rust fungal development on the leaves was then determined visually.

In this test, the plants which had been treated with 250 ppm of the compound from example 59 showed no infection, whereas the untreated plants were 90% infected.

1. A 7-amino-6-hetaryl-1,2,4-triazolo[1,5-a]pyrimidine compound of the formula I 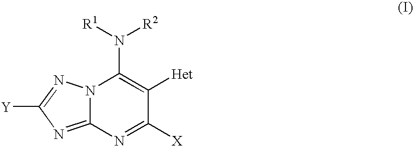
in which the substituents R¹, R², Het, X and Y are as defined below:
Het is a 6-membered heteroaromatic radical selected from the group consisting of pyridinyl, pyridazinyl, pyrazinyl, 1,2,4-triazinyl and 1,3,5-triazinyl, where the 6-membered heteroaromatic radical may have 1,2, 3 or 4 identical or different substituents L,
R¹, R² independently of one another are hydrogen, C₁-C₈-alkyl, C₁-C₈-haloalkyl, C₁-C₈-alkoxy, C₃-C₈-cycloalkyl, C₃-C₈-cycloalkoxy, C₅-C₁₀-bicycloalkyl, C₃-C₈-halocycloalkyl, C₂-C₈-alkenyl, C₂-C₈-alkenyloxy, C₄-C₁₀-alkadienyl, C₂-C₈-haloalkenyl, C₃-C₈-cycloalkenyl, C₃-C₈-halocycloalkenyl, C₂-C₈-alkynyl, C₂-C₈-alkynyloxy, C₂-C₈-haloalkynyl, NH₂, C₁-C₈-alkylamino, di-C₁-C₈-alkylamino, phenyl, naphthyl or a five- or six-membered saturated, partially unsaturated or aromatic heterocycle which contains one, two, three or four heteroatoms from the group consisting of O, N and S,
R¹ and R² together with the nitrogen atom to which they are attached may also form a five- or six-membered heterocyclyl or heteroaryl which is attached via N and may contain one, two or three further heteroatoms from the group consisting of O, N and S as ring members and may carry one or more substituents from the group consisting of halogen, C₁-C₆-alkyl, C₁-C₆-haloalkyl, C₂-C₆-alkenyl, C₂-C₆-haloalkenyl, C₁-C₆-alkoxy, C₁-C₆-alkoxycarbonyl, C₁-C₆-haloalkoxy, C₃-C₆-alkenyloxy, C₃-C₆-haloalkenyloxy and in which two substituents attached to adjacent ring atoms may be C₁-C₆-alkylene, oxy-C₂-C₄-alkylene or oxy-C₁-C₃-alkyleneoxy;
R¹ and R² may carry one, two, three or four identical or different groups R^{a}:
R^{a} is cyano, nitro, hydroxyl, carboxyl, C₁-C₆-alkyl, C₁-C₆-haloalkyl, C₁-C₆-alkylcarbonyl, C₃-C₆-cycloalkyl, C₁-C₆-alkoxy, C₁-C₆-haloalkoxy, C₁-C₆-alkoxycarbonyl, C₁-C₆-alkylthio, C₁-C₆-alkylamino, di-C₁-C₆-alkylamino, C₁-C₆-alkylaminocarbonyl, di-C₁-C₆-alkylaminocarbonyl, C₂-C₈-alkenyl, C₄-C₁₀-alkadienyl, C₂-C₈-haloalkenyl, C₃-C₈-cycloalkenyl, C₂-C₆-alkenyloxy, C₃-C₆-haloalkenyloxy, C₂-C₆-alkynyl, C₂-C₆-haloalkynyl, C₃-C₆-alkynyloxy, C₃-C₆-haloalkynyloxy, C₃-C₆-cycloalkoxy, C₃-C₆-cycloalkenyloxy, oxy-C₁-C₃-alkyleneoxy, phenyl, naphthyl, a five-, six-, seven-, eight-, nine- or ten-membered saturated, partially unsaturated or aromatic heterocycle which contains one, two, three or four heteroatoms from the group consisting of O, N and S,
where the aliphatic, alicyclic or aromatic groups in R^{a} for their part may be partially or fully halogenated or may carry one, two or three groups R^{b}:
R^{b} is cyano, nitro, hydroxyl, mercapto, amino, carboxyl, aminocarbonyl, aminothiocarbonyl, alkyl, haloalkyl, alkenyl, alkadienyl, alkenyloxy, alkynyloxy, alkoxy, haloalkoxy, alkylthio, alkylamino, dialkylamino, formyl, alkylcarbonyl, alkylsulfonyl, alkylsulfoxyl, alkoxycarbonyl, alkylcarbonyloxy, alkylaminocarbonyl, dialkylaminocarbonyl, alkylaminothiocarbonyl, dialkylaminothiocarbonyl, where the alkyl groups in these radicals contain 1 to 6 carbon atoms and the alkenyl, alkadienyl or alkynyl groups mentioned in these radicals contain 2 to 8 carbon atoms;
or one, two or three of the following radicals:
cycloalkyl, bicycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6, 7, 8, 9 or 10 ring members and the hetaryl radicals 5 or 6 ring members, where the cyclic systems may be partially or fully halogenated or substituted by alkyl or haloalkyl groups;
where R² may also be an organic radical which contains 3 to 13 carbon atoms and one or more silicon atoms and optionally 1 to 3 identical or different heteroatoms from the group consisting of oxygen, nitrogen and sulfur and which is unsubstituted or carries 1, 2, 3 or 4 identical or different substituents selected from the group consisting of halogen atoms and the substituents R^{a}, or R¹ and R² together with the nitrogen atom to which they are attached may be a heterocyclic ring which contains one or more silicon atoms and which is unsubstituted or carries 1, 2, 3 or 4 identical or different substituents selected from the group consisting of halogen atoms and the substituents R^{a};
X is hydrogen, OH, halogen, cyano, NR³R⁴, C₁-C₈-alkyl, C₁-C₈-alkoxy, C₁-C₈-alkylthio, C₁-C₈-alkylsulfinyl, C₁-C₈-alkylsulfonyl, C₂-C₈-alkenyl or C₂-C₈-alkynyl, where the 7 last-mentioned radicals may be partially or fully halogenated and may carry one, two or three substituents selected from the group consisting of nitro, cyano, C₁-C₂-alkoxy, C₁-C₄-alkoxycarbonyl, amino, C₁-C₄-alkylamino and di-C₁-C₄-alkylamino, and where R³ and R⁴ independently of one another have the meanings given for R¹ and R², respectively;
Y is hydrogen, halogen, cyano, C₁-C₄-alkyl, C₁-C₄-haloalkyl, C₁-C₄-alkoxy, C₁-C₄-alkylthio, C₁-C₄-alkylsulfinyl, C₁-C₄-alkylsulfonyl, formyl, C₁-C₄-alkylcarbonyl, C₁-C₄-alkoxycarbonyl, CONR³R⁴ or C₁-C₂-haloalkoxy; where
L is selected from the group consisting of halogen, cyano, hydroxyl, cyanato (OCN), nitro, C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoximinoalkyl, C₂-C₁₀-alkenyloximinoalkyl, C₂-C₁₀-alkynyloximinoalkyl, C₂-C₁₀-alkynylcarbonyl, C₃-C₆-cycloalkylcarbonyl, phenyl, a five-, six-, seven-, eight-, nine- or ten-membered saturated, partially unsaturated or aromatic heterocycle which contains one, two, three or four heteroatoms from the group consisting of O, N and S, where phenyl and the heterocycle may be unsubstituted or have 1, 2, 3 or 4 substituents selected from the group consisting of halogen and R^{a}, amino, NR⁵R⁶, NR⁵—(C═O)—R⁶, S(═O)ₙA¹, C(═O)A², C(═S)A², a group —C(═N—OR⁷)(NR⁸R⁹) and a group —C(═N—NR¹⁰R¹¹)(NR¹²R¹³),
where
R⁵, R⁶ independently of one another are selected from the group consisting of hydrogen, C₁-C₆-alkyl, C₂-C₁₀-alkenyl, C₂-C₁₀-alkynyl, C₃-C₆-cycloalkyl and C₃-C₆-cycloalkenyl, where the 5 last-mentioned radicals may be partially or fully halogenated or may carry one, two, three or four radicals selected from the group consisting of cyano, C₁-C₄-alkoximino, C₂-C₄-alkenyloximino, C₂-C₄-alkynyloximino and C₁-C₄-alkoxy;
A¹ is hydrogen, hydroxyl, C₁-C₈-alkyl, amino, C₁-C₈-alkylamino or di-(C₁-C₈-alkyl)amino;
n is 0, 1 or 2;
A² is C₂-C₈-alkenyl, C₁-C₈-alkoxy, C₁-C₆-haloalkoxy, C₂-C₁₀-alkenyloxy, C₂-C₁₀-alkynyloxy or one of the groups mentioned under A¹;
R⁷, R⁸, R⁹, R¹⁰, R¹¹, R¹² and R¹³ independently of one another are selected from the group consisting of hydrogen, C₁-C₆-alkyl, C₃-C₆-cycloalkyl, C₂-C₆-alkenyl and C₂-C₆-alkynyl, where the four last-mentioned radicals may have one, two, three, four, five or six radicals R^{a}; or
R⁸ and R⁹, R¹⁰ and R¹¹ and/or R¹² and R¹³ together with the nitrogen atom to which they are attached form a four-, five- or six-membered saturated or partially unsaturated ring which may carry one, two, three or four substituents independently of one another selected from the group R^{a};
or an agriculturally acceptable salt of the compound I.

2. The compound of the formula I according to claim 1 in which R¹ and R² are as defined below:
R¹ is C₁-C₆-alkyl, C₂-C₆-alkenyl, C₂-C₆-alkynyl, C₃-C₆-cycloalkyl which may be mono-, di-, tri- or tetrasubstituted by one or more selected from the group consisting of halogen, OH, C₁-C₄-alkoxy and C₁-C₄-alkyl, or C₁-C₈-haloalkyl and
R² is hydrogen or C₁-C₄-alkyl; or
R¹ and R² together with the nitrogen atom to which they are attached may also form a five- or six-membered saturated, monounsaturated or aromatic heterocycle which may carry one or two substituents from the group consisting of halogen, C₁-C₆-alkyl and C₁-C₆-haloalkyl.

3. The compound of the formula I according claim 1 in which X is halogen, cyano, C₁-C₄-alkyl, C₁-C₄-haloalkyl, C₁-C₄-alkoxy, C₁-C₄-alkylthio or C₁-C₂-haloalkoxy.

4. The compound of the formula I according to claim 3 in which X is halogen.

5. The compound of the formula I according to claim 3 in which X is CN, C₁-C₄-alkoxy or C₁-C₄-alkylthio.

6. The compound of the formula I according to claim 1 in which Het is pyridinyl which may optionally have 1, 2 or 3 substituents L.

7. The compound of the formula I according to claim 6 in which Het is 2-pyridinyl which has 1 or 2 substituents L.

8. The compound of the formula I according to claim 7 in which one of the substituents L is located in the 5-position of the pyridinyl ring.

9. The compound of the formula I according to claim 7 in which one of the substituents L is located in the 3-position of the pyridinyl ring.

10. The compound of the formula I according to claim 7 in which Het is a radical of the formula Het-1 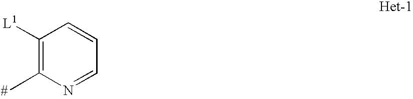
in which # is the point of attachment to the triazolopyrimidine unit and L¹ is chlorine, bromine, iodine, C₁-C₂-alkyl, C₁-C₂-alkoxy, methylthio, methylsulfinyl, methylsulfonyl, nitro or methoxymethyl.

11. The compound of the formula I according to claim 7 in which Het is a radical of the formula Het-2 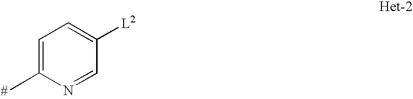
in which # is the point of attachment to the triazolopyrimidine unit and L² is fluorine, chlorine, bromine, iodine, cyano, nitro, C₁-C₂-alkyl, C₁-C₂-alkoxy, C₁-C₂-alkoxycarbonyl, CONH₂, C₁-C₂-alkylaminocarbonyl, C₁-C₂-alkylcarbonyl or C(S)NH₂.

12. The compound of the formula I according to claim 7 in which Het is a radical of the formula Het-3 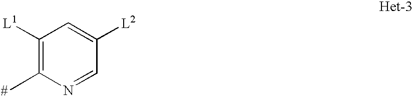
in which # is the point of attachment to the triazolopyrimidine unit,
L¹ is chlorine, bromine, iodine, C₁-C₂-alkyl, C₁-C₂-alkoxy, methylthio, methylsulfinyl, methylsulfonyl, nitro or methoxymethyl and
L² is fluorine, chlorine, bromine, iodine, cyano, nitro, C₁-C₂-alkyl, C₁-C₂-alkoxy, C₁-C₂-alkoxycarbonyl, CONH₂, C₁-C₂-alkylaminocarbonyl, C₁-C₂-alkylcarbonyl or C(S)NH₂.

13. The compound of the formula I according to claim 6 in which Het is 3-pyridinyl which optionally has 1 or 2 substituents L.

14. The compound of the formula I according to claim 13 in which Het is a radical of the formula Het-4, Het-5, Het-6, Het-7 or Het-8: 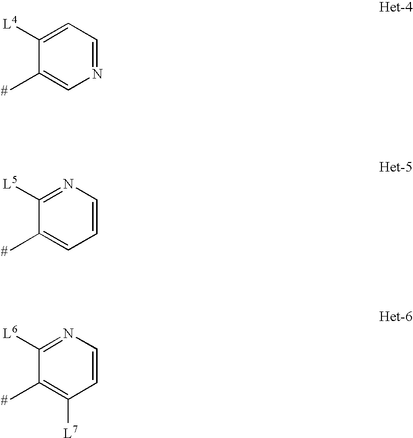 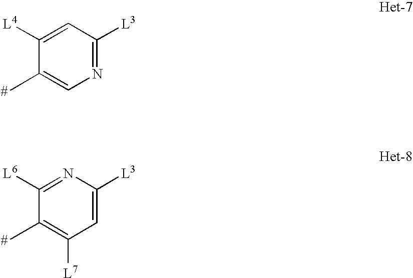
in which # is the point of attachment to the triazolopyrimidine unit and
L³ is fluorine, chlorine, bromine, iodine, cyano, nitro, C₁-C₂-alkyl, C₁-C₂-alkoxy, C₁-C₂-alkoxycarbonyl, CONH₂, C₁-C₂-alkylaminocarbonyl, C₁-C₂-alkylcarbonyl or C(S)NH₂,
L⁴ fluorine, chlorine, bromine, iodine, C₁-C₂-alkyl, C₁-C₂-alkoxy, methylthio, methylsulfinyl, methylsulfonyl, nitro or methoxymethyl,
L⁵ is fluorine, chlorine, bromine, iodine, C₁-C₂-alkyl, C₁-C₂-alkoxy, methylthio, methylsulfinyl, methylsulfonyl, nitro or methoxymethyl, and
L⁶ and L⁷ independently of one another have one of the following meanings: fluorine, chlorine, bromine, iodine, C₁-C₂-alkyl, C₁-C₂-alkoxy, methylthio, methylsulfinyl, methylsulfonyl, nitro or methoxymethyl

15. The compound of the formula I according to claim 6 in which Het is 4-pyridinyl which optionally has 1 or 2 substituents L.

16. The compound of the formula I according to claim 15 in which Het is a radical of the formula Het-9 or Het-10: 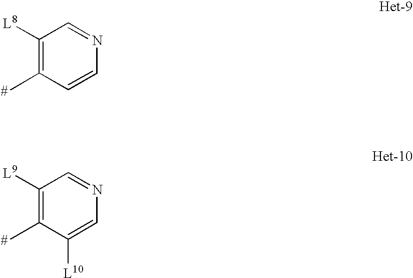
in which # is the point of attachment to the triazolopyrimidine unit and
L⁸ is fluorine, chlorine, bromine, iodine, C₁-C₂-alkyl, C₁-C₂-alkoxy, methylthio, methylsulfinyl, methylsulfonyl, nitro or methoxymethyl,
L⁹ is fluorine, chlorine, bromine, iodine, C₁-C₂-alkyl, C₁-C₂-alkoxy, methylthio, methylsulfinyl, methylsulfonyl, nitro or methoxymethyl, and
L¹⁰ is fluorine, chlorine, bromine, iodine, cyano, nitro, C₁-C₂-alkyl, C₁-C₂-alkoxy, C₁-C₂-alkoxycarbonyl, CONH₂, C₁-C₂-alkylaminocarbonyl, C₁-C₂-alkylcarbonyl or C(S)NH₂.

17. The compound of the formula I according to claim 1 in which Het is 2-pyrazinyl which optionally has 1, 2 or 3 substituents L.

18. The compound of the formula I according to claim 1 in which Het is 1,3,5-triazinyl which optionally has 1 or 2 substituents L.

19. The compound of the formula I according to claim 1 in which Het has 1, 2 or 3 substituents L independently of one another selected from the group consisting of halogen, cyano, nitro, NH₂, C₁-C₆-alkylamino, di-C₁-C₆-alkylamino, C₁-C₆-alkyl, C₁-C₆-haloalkyl, C₁-C₆-alkoxy, C₁-C₆-alkylamino, di-C₁-C₆-alkylamino, NH—C(O)—C₁-C₆-alkyl, a group C(S)A² and a group C(O)A².

20. (canceled)

21. A composition for controlling phytopathogenic fungi, which composition comprises at least one compound of the formula I according to claim 1 or an agriculturally acceptable salt of I and at least one solid or liquid carrier.

22. A method for controlling phytopathogenic fungi, which method comprises treating the fungi or the materials, plants, the soil or seed to be protected against fungal attack with an effective amount of at least one compound of the formula I according to claim 1 or an agriculturally acceptable salt of I.

23. Seed comprising a compound of the formula I according to claim 1 in an amount of from 1 to 1000 g per 100 kg.

24. A compound of the general formula II or III 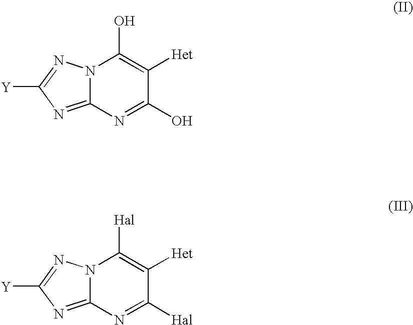
in which the substituents
Het is a 6-membered heteroaromatic radical selected from the group consisting of pyridinyl, pyridazinyl, pyrazinyl, 1,2,4-triazinyl and 1,3,5-triazinyl, where the 6-membered heteroaromatic radical may have 1, 2, 3 or 4 identical or different substituents L,
and
Y is hydrogen, halogen, cyano, C₁-C₄-alkyl C₁-C₄-haloalkyl, C₁-C₄-alkoxy, C₁-C₄-alkylthio, C₁-C₄-alkylsulfinyl, C₁-C₄-alkylsulfonyl, formyl, C₁-C₄-alkylcarbonyl, C₁-C₄-alkoxycarbonyl, CONR³R⁴ or C₁-C₂-haloalkoxy;
and Hal is halogen.

25. (canceled)

26. A composition for controlling phytopathogenic fungi, which composition comprises at least one compound of the formula II or III according to claim 24 or an agriculturally acceptable salt thereof and at least one solid or liquid carrier.

27. A method for controlling phytopathogenic fungi, which method comprises treating the fungi or the materials, plants, the soil or seed to be protected against fungal attack with an effective amount of at least one compound of the formula II or III according to claim 24 or an agriculturally acceptable salt thereof.

28. Seed comprising a compound of the formula II or III according to claim 24 in an amount of from 1 to 1000 g per 100 kg.

29. A compound of the formula VI 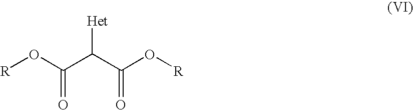
in which R is C₁-C₄-alkyl and Het is a 6-membered heteroaromatic radical selected from the group consisting of pyridinyl, pyridazinyl, pyrazinyl, 1,2,4-triazinyl and 1,3,5-triazinyl, where the 6-membered heteroaromatic radical has 1, 2 or 3 identical or different substituents L independently of one another selected from the group consisting of hydroxyl, cyanato (OCN), C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₁-C₈-alkoximinoalkyl, C₂-C₁₀-alkenyloximinoalkyl, C₂-C₁₀-alkynyloximinoalkyl, C₂-C₁₀-alkynylcarbonyl, C₃-C₆-cycloalkylcarbonyl, C(═O)A², C(═S)A², a group —C(═N—R⁷)(NR⁸R⁹) and a group —C(═N—NR¹⁰R¹¹)(NR¹²R¹³), where
A² is C₂-C₈-alkenyl, C₁-C₈-alkoxy, C₁-C₆-haloalkoxy, C₂-C₁₀-alkenyloxy, C₁-C₁₀-alkynyloxy or one of the groups mentioned under A¹;
R⁷, R⁸, R⁹, R¹⁰, R¹¹, R¹² and R¹³ independently of one another are selected from the group consisting of hydrogen, C₁-C₆-alkyl, C₃-C₆-cycloalkyl C₂-C₆-alkenyl and C₁-C₆-alkynyl, where the four last-mentioned radicals may have one, two, three, four, five or six radicals R^{a};
where Het may additionally also have 1, 2 or 3 further substituents selected from the group consisting of halogen and cyano,
provided that Het is other than 5-trifluoromethylpyridin-2-yl, 6-trifluoromethylpyridin-2-yl, 3-chloro-5-trifluoromethylpyridin-2-yl, 3-chloro-6-trifluoromethylpyridin-2-yl, 3-cyano-5-trifluoromethylpyridin-2-yl, 3-cyano-6-isopropylpyridin-2-yl, 4,6-dimethoxy-1,3,5-triazin-2-yl, 3-trifluoromethyl-4-(1,2,2,2-tetrafluoro-1-(trifluoromethyl)ethyl)pyridin-2-yl or 3,4,5-trifluoro-6-(1,2,2,2-tetrafluoro-1-(trifluoromethyl)ethyl)pyridin-2-yl.

30. A compound of the formula VIa, 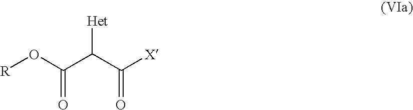
in which R is C₁-C₄-alkyl, X′ is C₁-C₈-alkyl, C₂-C₈-alkenyl or C₂-C₈-alkynyl or a corresponding halogenated radical and Het is a 6-membered heteroaromatic radical selected from the group consisting of pyridinyl, pyridazinyl, pyrazinyl, 1,2,4-triazinyl and 1,3,5-triazinyl, where the 6-membered heteroaromatic radical has 1, 2 or 3 substituents independently of one another selected from the group consisting of cyano, hydroxyl, cyanato (OCN), C₁-C₈-alkyl, C₂-C₁₀-alkenyl, C₂-C₁₀-alkynyl, C₁-C₆-haloalkyl, C₂-C₁₀-haloalkenyl, C₁-C₆-alkoxy, C₂-C₁₀-alkenyloxy, C₂-C₁₀-alkynyloxy, C₁-C₆-haloalkoxy, C₃-C₆-cycloalkyl, C₃-C₆-cycloalkenyl, C₃-C₆-cycloalkoxy, C₂-C₁₀-alkenyloxycarbonyl, C₂-C₁₀-alkynyloxycarbonyl, C₁-C₈-alkoximinoalkyl, C₂-C₁₀-alkenyloximinoalkyl, C₂-C₁₀-alkynyloximinoalkyl, C₁-C₈-alkylcarbonyl, C₂-C₁₀-alkenylcarbonyl, C₂-C₁₀-alkynylcarbonyl, C₃-C₆-cycloalkylcarbonyl, S(═O)ₙA¹, C(═O)A², C(═S)A², a group —C(═N—OR⁷)(NR⁸R⁹) and a group —C(═N—NR¹⁰R¹¹)(NR¹²R¹³), where A¹, A², R⁷, R⁸, R⁹, R¹⁰, R¹¹, R¹² and R¹³ are as defined above, where Het may additionally also have 1, 2 or 3 further halogen atoms as substituents L,
provided that Het is other than 5-ethoxycarbonylpyridin-2-yl, 2-methylpyridin-3-yl, 4-chloro-6-methoxy-1,3,5-triazin-2-yl or 4,6-dimethoxy-1,3,5-triazin-2-yl.
